SEQUENCE LISTING (309 sequences)

SEQ ID NO: 1 - protein, 207 aa, Homo sapiens
  REGION 1..207: note = misc_feature - Human (hCD3e) UniProt:P0776; CD3E_HUMAN T-cell surface glycoprotein CD3 epsilon chain
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 2 - protein, 198 aa, Macaca fascicularis
  REGION 1..198: note = misc_feature - Cynomolgus CD3e UniProt:Q95LI5; CD3E_MACFA T-cell surface glycoprotein CD3 epsilon chain
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 3 - protein, 189 aa, Mus musculus
  REGION 1..189: note = misc_feature - Murine CD3e UniProt: P22646; CD3E_MOUSE T-cell surface glycoprotein CD3 epsilon chain
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKERPPPVPNPDYEPIRKGQRDLYSGLNQRAV

SEQ ID NO: 4 - protein, 7 aa, synthetic construct
  REGION 1..7: note = Synthetic: MSP34
GFTFNTY

SEQ ID NO: 5 - protein, 7 aa, synthetic construct
  REGION 1..7: note = Synthetic: HSP34-HC4
GFTFNKY

SEQ ID NO: 6 - protein, 7 aa, synthetic construct
  REGION 1..7: note = Synthetic: hSP34-Hc3
GFTFNKY

SEQ ID NO: 7 - protein, 7 aa, synthetic construct
  REGION 1..7: note = Synthetic: hSP34-Hc2
GFTFNKY

SEQ ID NO: 8 - protein, 7 aa, synthetic construct
  REGION 1..7: note = Synthetic: hSP34-Hc1
GFTFNKY

SEQ ID NO: 9 - protein, 7 aa, synthetic construct
  REGION 1..7: note = Synthetic: SP34-Hc
GFTFNKY

SEQ ID NO: 10 - protein, 7 aa, synthetic construct
  REGION 1..7: note = Synthetic: 2037-B10
GFTFNKY

SEQ ID NO: 11 - protein, 7 aa, synthetic construct
  REGION 1..7: note = Synthetic: 2037-B02/A11/A02
GFTFNKY

SEQ ID NO: 12 - protein, 7 aa, synthetic construct
  REGION 1..7: note = Synthetic: 2037-B10_VH-Y108D
GFTFNKY

SEQ ID NO: 13 - protein, 7 aa, synthetic construct
  REGION 1..7: note = Synthetic: 2037-B10_VH-S107D
GFTFNKY

SEQ ID NO: 14 - protein, 7 aa, synthetic construct
  REGION 1..7: note = Synthetic: 2037-B10_VH-N106D
GFTFNKY

SEQ ID NO: 15 - protein, 7 aa, synthetic construct
  REGION 1..7: note = Synthetic: 2037-B10_VH-G105D
GFTFNKY

SEQ ID NO: 16 - protein, 7 aa, synthetic construct
  REGION 1..7: note = Synthetic: 2037-B10_VH-F104D
GFTFNKY

SEQ ID NO: 17 - protein, 7 aa, synthetic construct
  REGION 1..7: note = Synthetic: 2037-B10_VH-N103D
GFTFNKY

SEQ ID NO: 18 - protein, 7 aa, synthetic construct
  REGION 1..7: note = Synthetic: 2037-B10_VH-Y61D
GFTFNKY

SEQ ID NO: 19 - protein, 7 aa, synthetic construct
  REGION 1..7: note = Synthetic: 2037-B10_VH-A59D
GFTFNKY

SEQ ID NO: 20 - protein, 7 aa, synthetic construct
  REGION 1..7: note = Synthetic: 2037-B10_VH-N56D
GFTFNKY

SEQ ID NO: 21 - protein, 7 aa, synthetic construct
  REGION 1..7: note = Synthetic: 2037-B10_VH-Y55D
GFTFNKY

SEQ ID NO: 22 - protein, 7 aa, synthetic construct
  REGION 1..7: note = Synthetic: 2037-B10_VH-K54D
GFTFNKY

SEQ ID NO: 23 - protein, 7 aa, synthetic construct
  REGION 1..7: note = Synthetic: 2037-B10_VH-R52D
GFTFNKY

SEQ ID NO: 24 - protein, 7 aa, synthetic construct
  REGION 1..7: note = Synthetic: 2037-B10_VH-R50D
GFTFNKY

SEQ ID NO: 25 - protein, 7 aa, synthetic construct
  REGION 1..7: note = Synthetic: 2037-B10_VH-A33D
GFTFNKY

SEQ ID NO: 26 - protein, 7 aa, synthetic construct
  REGION 1..7: note = Synthetic: 2037-B10_VH-K31D
GFTFNKY

SEQ ID NO: 27 - protein, 7 aa, synthetic construct
  REGION 1..7: note = Synthetic: 2037-B10_VH-N30D
GFTFDKY

SEQ ID NO: 28 - protein, 7 aa, synthetic construct
  REGION 1..7: note = Synthetic: 2037-B10_VH-F29D
GFTDNKY

SEQ ID NO: 29 - protein, 7 aa, synthetic construct
  REGION 1..7: note = Synthetic: 2037-B10_VH-F27D
GDTFNKY

SEQ ID NO: 30 - protein, 5 aa, synthetic construct
  REGION 1..5: note = Synthetic: MSP34
TYAMN

SEQ ID NO: 31 - protein, 5 aa, synthetic construct
  REGION 1..5: note = Synthetic: HSP34-HC4
KYAMN

SEQ ID NO: 32 - protein, 5 aa, synthetic construct
  REGION 1..5: note = Synthetic: hSP34-Hc3
KYAMN

SEQ ID NO: 33 - protein, 5 aa, synthetic construct
  REGION 1..5: note = Synthetic: hSP34-Hc2
KYAMN

SEQ ID NO: 34 - protein, 5 aa, synthetic construct
  REGION 1..5: note = Synthetic: hSP34-Hc1
KYAMN

SEQ ID NO: 35 - protein, 5 aa, synthetic construct
  REGION 1..5: note = Synthetic: SP34-Hc
KYAMN

SEQ ID NO: 36 - protein, 5 aa, synthetic construct
  REGION 1..5: note = Synthetic: 2037-B10
KYAMN

SEQ ID NO: 37 - protein, 5 aa, synthetic construct
  REGION 1..5: note = Synthetic: 2037-B02/A11/A02
KYAMN

SEQ ID NO: 38 - protein, 5 aa, synthetic construct
  REGION 1..5: note = Synthetic: 2037-B10_VH-Y108D
KYAMN

SEQ ID NO: 39 - protein, 5 aa, synthetic construct
  REGION 1..5: note = Synthetic: 2037-B10_VH-S107D
KYAMN

SEQ ID NO: 40 - protein, 5 aa, synthetic construct
  REGION 1..5: note = Synthetic: 2037-B10_VH-N106D
KYAMN

SEQ ID NO: 41 - protein, 5 aa, synthetic construct
  REGION 1..5: note = Synthetic: 2037-B10_VH-G105D
KYAMN

SEQ ID NO: 42 - protein, 5 aa, synthetic construct
  REGION 1..5: note = Synthetic: 2037-B10_VH-F104D
KYAMN

SEQ ID NO: 43 - protein, 5 aa, synthetic construct
  REGION 1..5: note = Synthetic: 2037-B10_VH-N103D
KYAMN

SEQ ID NO: 44 - protein, 5 aa, synthetic construct
  REGION 1..5: note = Synthetic: 2037-B10_VH-Y61D
KYAMN

SEQ ID NO: 45 - protein, 5 aa, synthetic construct
  REGION 1..5: note = Synthetic: 2037-B10_VH-A59D
KYAMN

SEQ ID NO: 46 - protein, 5 aa, synthetic construct
  REGION 1..5: note = Synthetic: 2037-B10_VH-N56D
KYAMN

SEQ ID NO: 47 - protein, 5 aa, synthetic construct
  REGION 1..5: note = Synthetic: 2037-B10_VH-Y55D
KYAMN

SEQ ID NO: 48 - protein, 5 aa, synthetic construct
  REGION 1..5: note = Synthetic: 2037-B10_VH-K54D
KYAMN

SEQ ID NO: 49 - protein, 5 aa, synthetic construct
  REGION 1..5: note = Synthetic: 2037-B10_VH-R52D
KYAMN

SEQ ID NO: 50 - protein, 5 aa, synthetic construct
  REGION 1..5: note = Synthetic: 2037-B10_VH-R50D
KYAMN

SEQ ID NO: 51 - protein, 5 aa, synthetic construct
  REGION 1..5: note = Synthetic: 2037-B10_VH-A33D
KYDMN

SEQ ID NO: 52 - protein, 5 aa, synthetic construct
  REGION 1..5: note = Synthetic: 2037-B10_VH-K31D
KYAMN

SEQ ID NO: 53 - protein, 5 aa, synthetic construct
  REGION 1..5: note = Synthetic: 2037-B10_VH-N30D
KYAMN

SEQ ID NO: 54 - protein, 5 aa, synthetic construct
  REGION 1..5: note = Synthetic: 2037-B10_VH-F29D
KYAMN

SEQ ID NO: 55 - protein, 5 aa, synthetic construct
  REGION 1..5: note = Synthetic: 2037-B10_VH-F27D
KYAMN

SEQ ID NO: 56 - protein, 8 aa, synthetic construct
  REGION 1..8: note = Synthetic: MSP34
RSKYNNYA

SEQ ID NO: 57 - protein, 8 aa, synthetic construct
  REGION 1..8: note = Synthetic: HSP34-HC4
RSKYNNYA

SEQ ID NO: 58 - protein, 8 aa, synthetic construct
  REGION 1..8: note = Synthetic: hSP34-Hc3
RSKYNNYA

SEQ ID NO: 59 - protein, 8 aa, synthetic construct
  REGION 1..8: note = Synthetic: hSP34-Hc2
RSKYNNYA

SEQ ID NO: 60 - protein, 8 aa, synthetic construct
  REGION 1..8: note = Synthetic: hSP34-Hc1
RSKYNNYA

SEQ ID NO: 61 - protein, 8 aa, synthetic construct
  REGION 1..8: note = Synthetic: SP34-Hc
RSKYNNYA

SEQ ID NO: 62 - protein, 8 aa, synthetic construct
  REGION 1..8: note = Synthetic: 2037-B10
RTKYNNYA

SEQ ID NO: 63 - protein, 8 aa, synthetic construct
  REGION 1..8: note = Synthetic: 2037-B02/A11/A02
RSKYNNYA

SEQ ID NO: 64 - protein, 8 aa, synthetic construct
  REGION 1..8: note = Synthetic: 2037-B10_VH-Y108D
RTKYNNYA

SEQ ID NO: 65 - protein, 8 aa, synthetic construct
  REGION 1..8: note = Synthetic: 2037-B10_VH-S107D
RTKYNNYA

SEQ ID NO: 66 - protein, 8 aa, synthetic construct
  REGION 1..8: note = Synthetic: 2037-B10_VH-N106D
RTKYNNYA

SEQ ID NO: 67 - protein, 8 aa, synthetic construct
  REGION 1..8: note = Synthetic: 2037-B10_VH-G105D
RTKYNNYA

SEQ ID NO: 68 - protein, 8 aa, synthetic construct
  REGION 1..8: note = Synthetic: 2037-B10_VH-F104D
RTKYNNYA

SEQ ID NO: 69 - protein, 8 aa, synthetic construct
  REGION 1..8: note = Synthetic: 2037-B10_VH-N103D
RTKYNNYA

SEQ ID NO: 70 - protein, 8 aa, synthetic construct
  REGION 1..8: note = Synthetic: 2037-B10_VH-Y61D
RTKYNNYA

SEQ ID NO: 71 - protein, 8 aa, synthetic construct
  REGION 1..8: note = Synthetic: 2037-B10_VH-A59D
RTKYNNYA

SEQ ID NO: 72 - protein, 8 aa, synthetic construct
  REGION 1..8: note = Synthetic: 2037-B10_VH-N56D
RTKYDNYA

SEQ ID NO: 73 - protein, 8 aa, synthetic construct
  REGION 1..8: note = Synthetic: 2037-B10_VH-Y55D
RTKDNNYA

SEQ ID NO: 74 - protein, 8 aa, synthetic construct
  REGION 1..8: note = Synthetic: 2037-B10_VH-K54D
RTDYNNYA

SEQ ID NO: 75 - protein, 8 aa, synthetic construct
  REGION 1..8: note = Synthetic: 2037-B10_VH-R52D
DTKYNNYA

SEQ ID NO: 76 - protein, 8 aa, synthetic construct
  REGION 1..8: note = Synthetic: 2037-B10_VH-R50D
RTKYNNYA

SEQ ID NO: 77 - protein, 8 aa, synthetic construct
  REGION 1..8: note = Synthetic: 2037-B10_VH-A33D
RTKYNNYA

SEQ ID NO: 78 - protein, 8 aa, synthetic construct
  REGION 1..8: note = Synthetic: 2037-B10_VH-K31D
RTKYNNYA

SEQ ID NO: 79 - protein, 8 aa, synthetic construct
  REGION 1..8: note = Synthetic: 2037-B10_VH-N30D
RTKYNNYA

SEQ ID NO: 80 - protein, 8 aa, synthetic construct
  REGION 1..8: note = Synthetic: 2037-B10_VH-F29D
RTKYNNYA

SEQ ID NO: 81 - protein, 8 aa, synthetic construct
  REGION 1..8: note = Synthetic: 2037-B10_VH-F27D
RTKYNNYA

SEQ ID NO: 82 - protein, 19 aa, synthetic construct
  REGION 1..19: note = Synthetic: MSP34
RIRSKYNNYATYYADSVKD

SEQ ID NO: 83 - protein, 19 aa, synthetic construct
  REGION 1..19: note = Synthetic: HSP34-HC4
RIRSKYNNYATYYADSVKD

SEQ ID NO: 84 - protein, 19 aa, synthetic construct
  REGION 1..19: note = Synthetic: hSP34-Hc3
RIRSKYNNYATYYADSVKD

SEQ ID NO: 85 - protein, 19 aa, synthetic construct
  REGION 1..19: note = Synthetic: hSP34-Hc2
RIRSKYNNYATYYADSVKD

SEQ ID NO: 86 - protein, 19 aa, synthetic construct
  REGION 1..19: note = Synthetic: hSP34-Hc1
RIRSKYNNYATYYADSVKD

SEQ ID NO: 87 - protein, 19 aa, synthetic construct
  REGION 1..19: note = Synthetic: SP34-Hc
RIRSKYNNYATYYADSVKD

SEQ ID NO: 88 - protein, 19 aa, synthetic construct
  REGION 1..19: note = Synthetic: 2037-B10
RIRTKYNNYATYYADSVKD

SEQ ID NO: 89 - protein, 19 aa, synthetic construct
  REGION 1..19: note = Synthetic: 2037-B02/A11/A02
RIRSKYNNYATYYADSVKD

SEQ ID NO: 90 - protein, 19 aa, synthetic construct
  REGION 1..19: note = Synthetic: 2037-B10_VH-Y108D
RIRTKYNNYATYYADSVKD

SEQ ID NO: 91 - protein, 19 aa, synthetic construct
  REGION 1..19: note = Synthetic: 2037-B10_VH-S107D
RIRTKYNNYATYYADSVKD

SEQ ID NO: 92 - protein, 19 aa, synthetic construct
  REGION 1..19: note = Synthetic: 2037-B10_VH-N106D
RIRTKYNNYATYYADSVKD

SEQ ID NO: 93 - protein, 19 aa, synthetic construct
  REGION 1..19: note = Synthetic: 2037-B10_VH-G105D
RIRTKYNNYATYYADSVKD

SEQ ID NO: 94 - protein, 19 aa, synthetic construct
  REGION 1..19: note = Synthetic: 2037-B10_VH-F104D
RIRTKYNNYATYYADSVKD

SEQ ID NO: 95 - protein, 19 aa, synthetic construct
  REGION 1..19: note = Synthetic: 2037-B10_VH-N103D
RIRTKYNNYATYYADSVKD

SEQ ID NO: 96 - protein, 19 aa, synthetic construct
  REGION 1..19: note = Synthetic: 2037-B10_VH-Y61D
RIRTKYNNYATDYADSVKD

SEQ ID NO: 97 - protein, 19 aa, synthetic construct
  REGION 1..19: note = Synthetic: 2037-B10_VH-A59D
RIRTKYNNYDTYYADSVKD

SEQ ID NO: 98 - protein, 19 aa, synthetic construct
  REGION 1..19: note = Synthetic: 2037-B10_VH-N56D
RIRTKYDNYATYYADSVKD

SEQ ID NO: 99 - protein, 19 aa, synthetic construct
  REGION 1..19: note = Synthetic: 2037-B10_VH-Y55D
RIRTKDNNYATYYADSVKD

SEQ ID NO: 100 - protein, 19 aa, synthetic construct
  REGION 1..19: note = Synthetic: 2037-B10_VH-K54D
RIRTDYNNYATYYADSVKD

SEQ ID NO: 101 - protein, 19 aa, synthetic construct
  REGION 1..19: note = Synthetic: 2037-B10_VH-R52D
RIDTKYNNYATYYADSVKD

SEQ ID NO: 102 - protein, 19 aa, synthetic construct
  REGION 1..19: note = Synthetic: 2037-B10_VH-R50D
DIRTKYNNYATYYADSVKD

SEQ ID NO: 103 - protein, 19 aa, synthetic construct
  REGION 1..19: note = Synthetic: 2037-B10_VH-A33D
RIRTKYNNYATYYADSVKD

SEQ ID NO: 104 - protein, 19 aa, synthetic construct
  REGION 1..19: note = Synthetic: 2037-B10_VH-K31D
RIRTKYNNYATYYADSVKD

SEQ ID NO: 105 - protein, 19 aa, synthetic construct
  REGION 1..19: note = Synthetic: 2037-B10_VH-N30D
RIRTKYNNYATYYADSVKD

SEQ ID NO: 106 - protein, 19 aa, synthetic construct
  REGION 1..19: note = Synthetic: 2037-B10_VH-F29D
RIRTKYNNYATYYADSVKD

SEQ ID NO: 107 - protein, 19 aa, synthetic construct
  REGION 1..19: note = Synthetic: 2037-B10_VH-F27D
RIRTKYNNYATYYADSVKD

SEQ ID NO: 108 - protein, 14 aa, synthetic construct
  REGION 1..14: note = Synthetic: MSP34
HGNFGNSYVSWFAY

SEQ ID NO: 109 - protein, 14 aa, synthetic construct
  REGION 1..14: note = Synthetic: HSP34-HC4
HGNFGNSYISYWAY

SEQ ID NO: 110 - protein, 14 aa, synthetic construct
  REGION 1..14: note = Synthetic: hSP34-Hc3
HGNFGNSYISYWAY

SEQ ID NO: 111 - protein, 14 aa, synthetic construct
  REGION 1..14: note = Synthetic: hSP34-Hc2
HGNFGNSYISYWAY

SEQ ID NO: 112 - protein, 14 aa, synthetic construct
  REGION 1..14: note = Synthetic: hSP34-Hc1
HGNFGNSYISYWAY

SEQ ID NO: 113 - protein, 14 aa, synthetic construct
  REGION 1..14: note = Synthetic: SP34-Hc
HGNFGNSYISYWAY

SEQ ID NO: 114 - protein, 14 aa, synthetic construct
  REGION 1..14: note = Synthetic: 2037-B10
HGNFGNSYISYWAY

SEQ ID NO: 115 - protein, 14 aa, synthetic construct
  REGION 1..14: note = Synthetic: 2037-B02/A11/A02
HGNFGNSYISYWAY

SEQ ID NO: 116 - protein, 14 aa, synthetic construct
  REGION 1..14: note = Synthetic: 2037-B10_VH-Y108D
HGNFGNSDISYWAY

SEQ ID NO: 117 - protein, 14 aa, synthetic construct
  REGION 1..14: note = Synthetic: 2037-B10_VH-S107D
HGNFGNDYISYWAY

SEQ ID NO: 118 - protein, 14 aa, synthetic construct
  REGION 1..14: note = Synthetic: 2037-B10_VH-N106D
HGNFGDSYISYWAY

SEQ ID NO: 119 - protein, 14 aa, synthetic construct
  REGION 1..14: note = Synthetic: 2037-B10_VH-G105D
HGNFDNSYISYWAY

SEQ ID NO: 120 - protein, 14 aa, synthetic construct
  REGION 1..14: note = Synthetic: 2037-B10_VH-F104D
HGNDGNSYISYWAY

SEQ ID NO: 121 - protein, 14 aa, synthetic construct
  REGION 1..14: note = Synthetic: 2037-B10_VH-N103D
HGDFGNSYISYWAY

SEQ ID NO: 122 - protein, 14 aa, synthetic construct
  REGION 1..14: note = Synthetic: 2037-B10_VH-Y61D
HGNFGNSYISYWAY

SEQ ID NO: 123 - protein, 14 aa, synthetic construct
  REGION 1..14: note = Synthetic: 2037-B10_VH-A59D
HGNFGNSYISYWAY

SEQ ID NO: 124 - protein, 14 aa, synthetic construct
  REGION 1..14: note = Synthetic: 2037-B10_VH-N56D
HGNFGNSYISYWAY

SEQ ID NO: 125 - protein, 14 aa, synthetic construct
  REGION 1..14: note = Synthetic: 2037-B10_VH-Y55D
HGNFGNSYISYWAY

SEQ ID NO: 126 - protein, 14 aa, synthetic construct
  REGION 1..14: note = Synthetic: 2037-B10_VH-K54D
HGNFGNSYISYWAY

SEQ ID NO: 127 - protein, 14 aa, synthetic construct
  REGION 1..14: note = Synthetic: 2037-B10_VH-R52D
HGNFGNSYISYWAY

SEQ ID NO: 128 - protein, 14 aa, synthetic construct
  REGION 1..14: note = Synthetic: 2037-B10_VH-R50D
HGNFGNSYISYWAY

SEQ ID NO: 129 - protein, 14 aa, synthetic construct
  REGION 1..14: note = Synthetic: 2037-B10_VH-A33D
HGNFGNSYISYWAY

SEQ ID NO: 130 - protein, 14 aa, synthetic construct
  REGION 1..14: note = Synthetic: 2037-B10_VH-K31D
HGNFGNSYISYWAY

SEQ ID NO: 131 - protein, 14 aa, synthetic construct
  REGION 1..14: note = Synthetic: 2037-B10_VH-N30D
HGNFGNSYISYWAY

SEQ ID NO: 132 - protein, 14 aa, synthetic construct
  REGION 1..14: note = Synthetic: 2037-B10_VH-F29D
HGNFGNSYISYWAY

SEQ ID NO: 133 - protein, 14 aa, synthetic construct
  REGION 1..14: note = Synthetic: 2037-B10_VH-F27D
HGNFGNSYISYWAY

SEQ ID NO: 134 - protein, 14 aa, synthetic construct
  REGION 1..14: note = Synthetic: mSP34
RSSTGAVTTSNYAN

SEQ ID NO: 135 - protein, 14 aa, synthetic construct
  REGION 1..14: note = Synthetic: hSP34-Lc4
GSSTGAVTSGYYPN

SEQ ID NO: 136 - protein, 14 aa, synthetic construct
  REGION 1..14: note = Synthetic: hSP34-Lc3
GSSTGAVTSGYYPN

SEQ ID NO: 137 - protein, 14 aa, synthetic construct
  REGION 1..14: note = Synthetic: hSP34-Lc2
GSSTGAVTSGYYPN

SEQ ID NO: 138 - protein, 14 aa, synthetic construct
  REGION 1..14: note = Synthetic: hSP34-Lc1
GSSTGAVTSGYYPN

SEQ ID NO: 139 - protein, 14 aa, synthetic construct
  REGION 1..14: note = Synthetic: SP34-LC
GSSTGAVTSGYYPN

SEQ ID NO: 140 - protein, 14 aa, synthetic construct
  REGION 1..14: note = Synthetic: 2037-B02
GSSTGAVTSGYYPN

SEQ ID NO: 141 - protein, 14 aa, synthetic construct
  REGION 1..14: note = Synthetic: 2037-A11
GSSTGAVTSGYYPN

SEQ ID NO: 142 - protein, 14 aa, synthetic construct
  REGION 1..14: note = Synthetic: 2037-A02/B10
GSSTGAVTSGYYPN

SEQ ID NO: 143 - protein, 14 aa, synthetic construct
  REGION 1..14: note = Synthetic: 2037-B10_R97D
GSSTGAVTSGYYPN

SEQ ID NO: 144 - protein, 14 aa, synthetic construct
  REGION 1..14: note = Synthetic: 2037-B10_N96D
GSSTGAVTSGYYPN

SEQ ID NO: 145 - protein, 14 aa, synthetic construct
  REGION 1..14: note = Synthetic: 2037-B10_S95D
GSSTGAVTSGYYPN

SEQ ID NO: 146 - protein, 14 aa, synthetic construct
  REGION 1..14: note = Synthetic: 2037-B10_Y94D
GSSTGAVTSGYYPN

SEQ ID NO: 147 - protein, 14 aa, synthetic construct
  REGION 1..14: note = Synthetic: 2037-B10_W93D
GSSTGAVTSGYYPN

SEQ ID NO: 148 - protein, 14 aa, synthetic construct
  REGION 1..14: note = Synthetic: 2037-B10-Lc10
GSSTGAVTSGYYPN

SEQ ID NO: 149 - protein, 14 aa, synthetic construct
  REGION 1..14: note = Synthetic: 2037-B10-Lc9
GSSTGAVTSGYYPN

SEQ ID NO: 150 - protein, 14 aa, synthetic construct
  REGION 1..14: note = Synthetic: 2037-B10-Lc8
GSSTGAVTSGYYPN

SEQ ID NO: 151 - protein, 14 aa, synthetic construct
  REGION 1..14: note = Synthetic: 2037-B10-Lc7
GSSTGAVTSGYYPN

SEQ ID NO: 152 - protein, 14 aa, synthetic construct
  REGION 1..14: note = Synthetic: 2037-B10-Lc6
GSSTGAVTSGYYPN

SEQ ID NO: 153 - protein, 14 aa, synthetic construct
  REGION 1..14: note = Synthetic: 2037-B10-Lc5
GSSTGAVTSGYYPN

SEQ ID NO: 154 - protein, 14 aa, synthetic construct
  REGION 1..14: note = Synthetic: 2037-B10-Lc4
GSSTGAVTSGYYPN

SEQ ID NO: 155 - protein, 14 aa, synthetic construct
  REGION 1..14: note = Synthetic: 2037-B10-Lc3
GSSTGAVTSGYYPN

SEQ ID NO: 156 - protein, 14 aa, synthetic construct
  REGION 1..14: note = Synthetic: 2037-B10-Lc2
GSSTGAVTSGYYPN

SEQ ID NO: 157 - protein, 14 aa, synthetic construct
  REGION 1..14: note = Synthetic: 2037-B10-Lc1
GSSTGAVTSGYYPN

SEQ ID NO: 158 - protein, 7 aa, synthetic construct
  REGION 1..7: note = Synthetic: mSP34
GTNKRAP

SEQ ID NO: 159 - protein, 7 aa, synthetic construct
  REGION 1..7: note = Synthetic: hSP34-Lc4
GTKFLAP

SEQ ID NO: 160 - protein, 7 aa, synthetic construct
  REGION 1..7: note = Synthetic: hSP34-Lc3
GTKFLAP

SEQ ID NO: 161 - protein, 7 aa, synthetic construct
  REGION 1..7: note = Synthetic: hSP34-Lc2
GTKFLAP

SEQ ID NO: 162 - protein, 7 aa, synthetic construct
  REGION 1..7: note = Synthetic: hSP34-Lc1
GTKFLAP

SEQ ID NO: 163 - protein, 7 aa, synthetic construct
  REGION 1..7: note = Synthetic: SP34-LC
GTKFLAP

SEQ ID NO: 164 - protein, 7 aa, synthetic construct
  REGION 1..7: note = Synthetic: 2037-B02
GTKFLAP

SEQ ID NO: 165 - protein, 7 aa, synthetic construct
  REGION 1..7: note = Synthetic: 2037-A11
GTKFLAP

SEQ ID NO: 166 - protein, 7 aa, synthetic construct
  REGION 1..7: note = Synthetic: 2037-A02/B10
GTKFLAP

SEQ ID NO: 167 - protein, 7 aa, synthetic construct
  REGION 1..7: note = Synthetic: 2037-B10_R97D
GTKFLAP

SEQ ID NO: 168 - protein, 7 aa, synthetic construct
  REGION 1..7: note = Synthetic: 2037-B10_N96D
GTKFLAP

SEQ ID NO: 169 - protein, 7 aa, synthetic construct
  REGION 1..7: note = Synthetic: 2037-B10_S95D
GTKFLAP

SEQ ID NO: 170 - protein, 7 aa, synthetic construct
  REGION 1..7: note = Synthetic: 2037-B10_Y94D
GTKFLAP

SEQ ID NO: 171 - protein, 7 aa, synthetic construct
  REGION 1..7: note = Synthetic: 2037-B10_W93D
GTKFLAP

SEQ ID NO: 172 - protein, 7 aa, synthetic construct
  REGION 1..7: note = Synthetic: 2037-B10-Lc10
GTKFLAP

SEQ ID NO: 173 - protein, 7 aa, synthetic construct
  REGION 1..7: note = Synthetic: 2037-B10-Lc9
GTKFLAP

SEQ ID NO: 174 - protein, 7 aa, synthetic construct
  REGION 1..7: note = Synthetic: 2037-B10-Lc8
GTKFLAP

SEQ ID NO: 175 - protein, 7 aa, synthetic construct
  REGION 1..7: note = Synthetic: 2037-B10-Lc7
GTKFLAP

SEQ ID NO: 176 - protein, 7 aa, synthetic construct
  REGION 1..7: note = Synthetic: 2037-B10-Lc6
GTKFLAP

SEQ ID NO: 177 - protein, 7 aa, synthetic construct
  REGION 1..7: note = Synthetic: 2037-B10-Lc5
GTKFLAP

SEQ ID NO: 178 - protein, 7 aa, synthetic construct
  REGION 1..7: note = Synthetic: 2037-B10-Lc4
GTKFLAP

SEQ ID NO: 179 - protein, 7 aa, synthetic construct
  REGION 1..7: note = Synthetic: 2037-B10-Lc3
GTKFLAP

SEQ ID NO: 180 - protein, 7 aa, synthetic construct
  REGION 1..7: note = Synthetic: 2037-B10-Lc2
GTKFLAP

SEQ ID NO: 181 - protein, 7 aa, synthetic construct
  REGION 1..7: note = Synthetic: 2037-B10-Lc1
GTKFLAP

SEQ ID NO: 182 - protein, 9 aa, synthetic construct
  REGION 1..9: note = Synthetic: mSP34
ALWYSNLWV

SEQ ID NO: 183 - protein, 9 aa, synthetic construct
  REGION 1..9: note = Synthetic: hSP34-Lc4
ALWYSNRWV

SEQ ID NO: 184 - protein, 9 aa, synthetic construct
  REGION 1..9: note = Synthetic: hSP34-Lc3
ALWYSNRWV

SEQ ID NO: 185 - protein, 9 aa, synthetic construct
  REGION 1..9: note = Synthetic: hSP34-Lc2
ALWYSNRWV

SEQ ID NO: 186 - protein, 9 aa, synthetic construct
  REGION 1..9: note = Synthetic: hSP34-Lc1
ALWYSNRWV

SEQ ID NO: 187 - protein, 9 aa, synthetic construct
  REGION 1..9: note = Synthetic: SP34-LC
ALWYSNRWV

SEQ ID NO: 188 - protein, 9 aa, synthetic construct
  REGION 1..9: note = Synthetic: 2037-B02
ALWYSNRWV

SEQ ID NO: 189 - protein, 9 aa, synthetic construct
  REGION 1..9: note = Synthetic: 2037-A11
ALWFSNRWV

SEQ ID NO: 190 - protein, 9 aa, synthetic construct
  REGION 1..9: note = Synthetic: 2037-A02/B10
ALWYSNRWV

SEQ ID NO: 191 - protein, 9 aa, synthetic construct
  REGION 1..9: note = Synthetic: 2037-B10_R97D
ALWYSNDWV

SEQ ID NO: 192 - protein, 9 aa, synthetic construct
  REGION 1..9: note = Synthetic: 2037-B10_N96D
ALWYSDRWV

SEQ ID NO: 193 - protein, 9 aa, synthetic construct
  REGION 1..9: note = Synthetic: 2037-B10_S95D
ALWYDNRWV

SEQ ID NO: 194 - protein, 9 aa, synthetic construct
  REGION 1..9: note = Synthetic: 2037-B10_Y94D
ALWDSNRWV

SEQ ID NO: 195 - protein, 9 aa, synthetic construct
  REGION 1..9: note = Synthetic: 2037-B10_W93D
ALDYSNRWV

SEQ ID NO: 196 - protein, 9 aa, synthetic construct
  REGION 1..9: note = Synthetic: 2037-B10-Lc10
ALWYSNRWV

SEQ ID NO: 197 - protein, 9 aa, synthetic construct
  REGION 1..9: note = Synthetic: 2037-B10-Lc9
ALWYSNRWV

SEQ ID NO: 198 - protein, 9 aa, synthetic construct
  REGION 1..9: note = Synthetic: 2037-B10-Lc8
ALWYSNRWV

SEQ ID NO: 199 - protein, 9 aa, synthetic construct
  REGION 1..9: note = Synthetic: 2037-B10-Lc7
ALWYSNRWV

SEQ ID NO: 200 - protein, 9 aa, synthetic construct
  REGION 1..9: note = Synthetic: 2037-B10-Lc6
ALWYSNRWV

SEQ ID NO: 201 - protein, 9 aa, synthetic construct
  REGION 1..9: note = Synthetic: 2037-B10-Lc5
ALWYSNRWV

SEQ ID NO: 202 - protein, 9 aa, synthetic construct
  REGION 1..9: note = Synthetic: 2037-B10-Lc4
ALWYSNRWV

SEQ ID NO: 203 - protein, 9 aa, synthetic construct
  REGION 1..9: note = Synthetic: 2037-B10-Lc3
ALWYSNRWV

SEQ ID NO: 204 - protein, 9 aa, synthetic construct
  REGION 1..9: note = Synthetic: 2037-B10-Lc2
ALWYSNRWV

SEQ ID NO: 205 - protein, 9 aa, synthetic construct
  REGION 1..9: note = Synthetic: 2037-B10-Lc1
ALWYSNRWV

SEQ ID NO: 206 - protein, 125 aa, synthetic construct
  REGION 1..125: note = Synthetic: MSP34
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 207 - protein, 125 aa, synthetic construct
  REGION 1..125: note = Synthetic: HSP34-HC4
EVQLVESGGGLVQPGGSLKLSCAASGFTFNKYAMNWVRQASGKGLEWVARIRSKYNNYATYYADSVKDRFTISRDDSKNTAYLQMNSLKTEDTAVYYCVRHGNFGNSYISYWAYWGQGTLVTVSS

SEQ ID NO: 208 - protein, 125 aa, synthetic construct
  REGION 1..125: note = Synthetic: hSP34-Hc3
EVQLVESGGGLVQPGRSLRLSCTASGFTFNKYAMNWFRQAPGKGLEWVARIRSKYNNYATYYADSVKDRFTISRDDSKSIAYLQMNSLKTEDTAVYYCVRHGNFGNSYISYWAYWGQGTLVTVSS

SEQ ID NO: 209 - protein, 125 aa, synthetic construct
  REGION 1..125: note = Synthetic: hSP34-Hc2
EVQLLESGGGLVQPGGSLRLSCAASGFTFNKYAMNWVRQAPGKGLEWVARIRSKYNNYATYYADSVKDRFTISRDDSKNTAYLQMNSLRAEDTAVYYCVRHGNFGNSYISYWAYWGQGTLVTVSS

SEQ ID NO: 210 - protein, 125 aa, synthetic construct
  REGION 1..125: note = Synthetic: hSP34-Hc1
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTLVTVSS

SEQ ID NO: 211 - protein, 125 aa, synthetic construct
  REGION 1..125: note = Synthetic: SP34-Hc
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 212 - protein, 125 aa, synthetic construct
  REGION 1..125: note = Synthetic: 2037-B10
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGNSYISYWAYWGQGTPVTVSS

SEQ ID NO: 213 - protein, 125 aa, synthetic construct
  REGION 1..125: note = Synthetic: 2037-B02/A11/A02
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSS

SEQ ID NO: 214 - protein, 125 aa, synthetic construct
  REGION 1..125: note = Synthetic: 2037-B10_VH-Y108D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGNSDISYWAYWGQGTPVTVSS

SEQ ID NO: 215 - protein, 125 aa, synthetic construct
  REGION 1..125: note = Synthetic: 2037-B10_VH-S107D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGNDYISYWAYWGQGTPVTVSS

SEQ ID NO: 216 - protein, 125 aa, synthetic construct
  REGION 1..125: note = Synthetic: 2037-B10_VH-N106D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGDSYISYWAYWGQGTPVTVSS

SEQ ID NO: 217 - protein, 125 aa, synthetic construct
  REGION 1..125: note = Synthetic: 2037-B10_VH-G105D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFDNSYISYWAYWGQGTPVTVSS

SEQ ID NO: 218 - protein, 125 aa, synthetic construct
  REGION 1..125: note = Synthetic: 2037-B10_VH-F104D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNDGNSYISYWAYWGQGTPVTVSS

SEQ ID NO: 219 - protein, 125 aa, synthetic construct
  REGION 1..125: note = Synthetic: 2037-B10_VH-N103D
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGDFGNSYISYWAYWGQGTPVTVSS

SEQ ID NO: 220 - protein, 125 aa, synthetic construct
  REGION 1..125: note = Synthetic: 2037-B10_VH-Y61D
EVQLVESGGGLVKPGGSLRLSCAASGFTFNKYAMNWVRQAPGKGLEWVARIRTKYNNYATDYADSVKDRFTISRDDSKNTAYLQMNSLKTEDTAVYYCVRHGNFGNSYISYWAYWGQGTPVTVSS

SEQ ID NO: 221 - protein, 125 aa, synthetic construct
  REGION 1..125: note = Synthetic: 2037-B10_VH-A59D
EVQLVESGGGLVKPGGSLRLSCAASGFTFNKYAMNWVRQAPGKGLEWVARIRTKYNNYDTYYADSVKDRFTISRDDSKNTAYLQMNSLKTEDTAVYYCVRHGNFGNSYISYWAYWGQGTPVTVSS

SEQ ID NO: 222 - protein, 125 aa, synthetic construct
  REGION 1..125: note = Synthetic: 2037-B10_VH-N56D
EVQLVESGGGLVKPGGSLRLSCAASGFTFNKYAMNWVRQAPGKGLEWVARIRTKYDNYATYYADSVKDRFTISRDDSKNTAYLQMNSLKTEDTAVYYCVRHGNFGNSYISYWAYWGQGTPVTVSS

SEQ ID NO: 223 - protein, 125 aa, synthetic construct
  REGION 1..125: note = Synthetic: 2037-B10_VH-Y55D
EVQLVESGGGLVKPGGSLRLSCAASGFTFNKYAMNWVRQAPGKGLEWVARIRTKDNNYATYYADSVKDRFTISRDDSKNTAYLQMNSLKTEDTAVYYCVRHGNFGNSYISYWAYWGQGTPVTVSS

SEQ ID NO: 224 - protein, 125 aa, synthetic construct
  REGION 1..125: note = Synthetic: 2037-B10_VH-K54D
EVQLVESGGGLVKPGGSLRLSCAASGFTFNKYAMNWVRQAPGKGLEWVARIRTDYNNYATYYADSVKDRFTISRDDSKNTAYLQMNSLKTEDTAVYYCVRHGNFGNSYISYWAYWGQGTPVTVSS

SEQ ID NO: 225 - protein, 125 aa, synthetic construct
  REGION 1..125: note = Synthetic: 2037-B10_VH-R52D
EVQLVESGGGLVKPGGSLRLSCAASGFTFNKYAMNWVRQAPGKGLEWVARIDTKYNNYATYYADSVKDRFTISRDDSKNTAYLQMNSLKTEDTAVYYCVRHGNFGNSYISYWAYWGQGTPVTVSS

SEQ ID NO: 226 - protein, 125 aa, synthetic construct
  REGION 1..125: note = Synthetic: 2037-B10_VH-R50D
EVQLVESGGGLVKPGGSLRLSCAASGFTFNKYAMNWVRQAPGKGLEWVADIRTKYNNYATYYADSVKDRFTISRDDSKNTAYLQMNSLKTEDTAVYYCVRHGNFGNSYISYWAYWGQGTPVTVSS

SEQ ID NO: 227 - protein, 124 aa, synthetic construct
  REGION 1..124: note = Synthetic: 2037-B10_VH-A33D
EVQLVESGGGLVKPGGSLRLSCAASGFTFNKYDMNWVRQAPGKGLEWVARIRTKYNYATYYADSVKDRFTISRDDSKNTAYLQMNSLKTEDTAVYYCVRHGNFGNSYISYWAYWGQGTPVTVSS

SEQ ID NO: 228 - protein, 125 aa, synthetic construct
  REGION 1..125: note = Synthetic: 2037-B10_VH-K31D
EVQLVESGGGLVKPGGSLRLSCAASGFTFNDYAMNWVRQAPGKGLEWVARIRTKYNNYATYYADSVKDRFTISRDDSKNTAYLQMNSLKTEDTAVYYCVRHGNFGNSYISYWAYWGQGTPVTVSS

SEQ ID NO: 229 - protein, 125 aa, synthetic construct
  REGION 1..125: note = Synthetic: 2037-B10_VH-N30D
EVQLVESGGGLVKPGGSLRLSCAASGFTFDKYAMNWVRQAPGKGLEWVARIRTKYNNYATYYADSVKDRFTISRDDSKNTAYLQMNSLKTEDTAVYYCVRHGNFGNSYISYWAYWGQGTPVTVSS

SEQ ID NO: 230 - protein, 125 aa, synthetic construct
  REGION 1..125: note = Synthetic: 2037-B10_VH-F29D
EVQLVESGGGLVKPGGSLRLSCAASGFTDNKYAMNWVRQAPGKGLEWVARIRTKYNNYATYYADSVKDRFTISRDDSKNTAYLQMNSLKTEDTAVYYCVRHGNFGNSYISYWAYWGQGTPVTVSS

SEQ ID NO: 231 - protein, 125 aa, synthetic construct
  REGION 1..125: note = Synthetic: 2037-B10_VH-F27D
EVQLVESGGGLVKPGGSLRLSCAASGDTFNKYAMNWVRQAPGKGLEWVARIRTKYNNYATYYADSVKDRFTISRDDSKNTAYLQMNSLKTEDTAVYYCVRHGNFGNSYISYWAYWGQGTPVTVSS

SEQ ID NO: 232 - protein, 109 aa, synthetic construct
  REGION 1..109: note = Synthetic: mSP34-VL
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 233 - protein, 109 aa, synthetic construct
  REGION 1..109: note = Synthetic: hSP34-Lc4-VL
QTVVTQEPSFSVSPGGTVTLTCGSSTGAVTSGYYPNWYQQTPGQAPRGLIGGTKFLAPGVPDRFSGSLLGNKAALTITGAQADDESDYYCALWYSNRWVFGGGTQLTVT

SEQ ID NO: 234 - protein, 109 aa, synthetic construct
  REGION 1..109: note = Synthetic: hSP34-Lc3-VL
QTVVTQEPSLTVSPGGTVTLTCGSSTGAVTSGYYPNWFQQKPGQAPRGLIGGTKFLAPGTPARFSGSLLGGKAALTLSGVQPEDEAEYYCALWYSNRWVFGGGTQLTVT

SEQ ID NO: 235 - protein, 109 aa, synthetic construct
  REGION 1..109: note = Synthetic: hSP34-Lc2-VL
QSAVTQPRSVSGSPGQSVTISCGSSTGAVTSGYYPNWYQQHPGKAPKGMIGGTKFLAPGVPDRFSGSLLGNTASLTISGLQAEDEADYYCALWYSNRWVFGGGTQLTVT

SEQ ID NO: 236 - protein, 110 aa, synthetic construct
  REGION 1..110: note = Synthetic: hSP34-Lc1-VL
EIVVTQSPGTLSLSPGERATLSCGSSTGAVTSGYYPNWYQQKPGQAPRGLIGGTKFLAPGIPDRFSGSLLGTDATLTISRLEPEDFAVYYCALWYSNRWVFGQGTKLEIK

SEQ ID NO: 237 - protein, 109 aa, synthetic construct
  REGION 1..109: note = Synthetic: SP34-LC-VL
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTQLTVT

SEQ ID NO: 238 - protein, 109 aa, synthetic construct
  REGION 1..109: note = Synthetic: 2037-B02-VL
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGSGTQLTVT

SEQ ID NO: 239 - protein, 109 aa, synthetic construct
  REGION 1..109: note = Synthetic: 2037-A11-VL
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWFSNRWVFGSGTQLTVT

SEQ ID NO: 240 - protein, 109 aa, synthetic construct
  REGION 1..109: note = Synthetic: 2037-A02/B10-VL
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 241 - protein, 109 aa, synthetic construct
  REGION 1..109: note = Synthetic: 2037-B10_VL-R97D
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNDWVFGGGTQLTVT

SEQ ID NO: 242 - protein, 109 aa, synthetic construct
  REGION 1..109: note = Synthetic: 2037-B10_VL-N96D
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSDRWVFGGGTQLTVT

SEQ ID NO: 243 - protein, 109 aa, synthetic construct
  REGION 1..109: note = Synthetic: 2037-B10_VL-S95D
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYDNRWVFGGGTQLTVT

SEQ ID NO: 244 - protein, 109 aa, synthetic construct
  REGION 1..109: note = Synthetic: 2037-B10_VL-Y94D
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWDSNRWVFGGGTQLTVT

SEQ ID NO: 245 - protein, 109 aa, synthetic construct
  REGION 1..109: note = Synthetic: 2037-B10_VL-W93D
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALDYSNRWVFGGGTQLTVT

SEQ ID NO: 246 - protein, 109 aa, synthetic construct
  REGION 1..109: note = Synthetic: 2037-B10-Lc10-VL
DTQVTQSPSLSASVGDRVTITCGSSTGAVTSGYYPNWLQQKPGQAPRGLIGGTKFLAPGVPSRFSGSLLGGKAALTISSLQPEDFAEYYCALWYSNRWVFGGGTKVEIK

SEQ ID NO: 247 - protein, 109 aa, synthetic construct
  REGION 1..109: note = Synthetic: 2037-B10-Lc9-VL
DTQVTQSPSLSASVGDRVTITCGSSTGAVTSGYYPNWLQQKPGQAPRGLIGGTKFLAPGVPSRFSGSLLGGKAALTISSLQPEDFATYYCALWYSNRWVFGGGTKVEIK

SEQ ID NO: 248 - protein, 109 aa, synthetic construct
  REGION 1..109: note = Synthetic: 2037-B10-Lc8-VL
DTQVTQSPSLSASVGDRVTITCGSSTGAVTSGYYPNWLQQKPGQAPRGLIGGTKFLAPGVPSRFSGSLSGGKATLTISSLQPEDFATYYCALWYSNRWVFGGGTKVEIK

SEQ ID NO: 249 - protein, 109 aa, synthetic construct
  REGION 1..109: note = Synthetic: 2037-B10-Lc7-VL
DTQVTQSPSLSASVGDRVTITCGSSTGAVTSGYYPNWYQQKPGKAPKLLIGGTKFLAPGVPSRFSGSLSGGKATLTISSLQPEDFATYYCALWYSNRWVFGGGTKVEIK

SEQ ID NO: 250 - protein, 110 aa, synthetic construct
  REGION 1..110: note = Synthetic: 2037-B10-Lc6-VL
DIQMTQSPSSLSASVGDRVTITCGSSTGAVTSGYYPNWLQQKPGQAPRGLIGGTKFLAPGVPSRFSGSLSGGKATLTISSLQPEDFATYYCALWYSNRWVFGGGTKVEIK

SEQ ID NO: 251 - protein, 109 aa, synthetic construct
  REGION 1..109: note = Synthetic: 2037-B10-Lc5-VL
DTQVTQSPSLSASVGDRVTITCGSSTGAVTSGYYPNWLQQKPGQAPRGLIGGTKFLAPGVPSRFSGSGSGTDFTLTISSLQPEDFATYYCALWYSNRWVFGGGTKVEIK

SEQ ID NO: 252 - protein, 110 aa, synthetic construct
  REGION 1..110: note = Synthetic: 2037-B10-Lc4-VL
DIQMTQSPSSLSASVGDRVTITCGSSTGAVTSGYYPNWYQQKPGKAPKLLIGGTKFLAPGVPSRFSGSLSGGKATLTISSLQPEDFATYYCALWYSNRWVFGGGTKVEIK

SEQ ID NO: 253 - protein, 110 aa, synthetic construct
  REGION 1..110: note = Synthetic: 2037-B10-Lc3-VL
DIQMTQSPSSLSASVGDRVTITCGSSTGAVTSGYYPNWLQQKPGQAPRGLIGGTKFLAPGVPSRFSGSGSGTDFTLTISSLQPEDFATYYCALWYSNRWVFGGGTKVEIK

SEQ ID NO: 254 - protein, 109 aa, synthetic construct
  REGION 1..109: note = Synthetic: 2037-B10-Lc2-VL
DTQVTQSPSLSASVGDRVTITCGSSTGAVTSGYYPNWYQQKPGKAPKLLIGGTKFLAPGVPSRFSGSGSGTDFTLTISSLQPEDFATYYCALWYSNRWVFGGGTKVEIK

SEQ ID NO: 255 - protein, 110 aa, synthetic construct
  REGION 1..110: note = Synthetic: 2037-B10-Lc1-VL
DIQMTQSPSSLSASVGDRVTITCGSSTGAVTSGYYPNWYQQKPGKAPKLLIGGTKFLAPGVPSRFSGSGSGTDFTLTISSLQPEDFATYYCALWYSNRWVFGGGTKVEIK

SEQ ID NO: 256 - protein, 330 aa, Homo sapiens
  REGION 1..330: note = misc_feature - Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 107 aa, Homo sapiens [sequence omitted]

SEQ ID NO: 258 - protein, 106 aa, Homo sapiens
  REGION 1..106: note = misc_feature - Human Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 259 - protein, 228 aa, synthetic construct
  REGION 1..228: note = Synthetic: Fc-knob
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 20 aa, synthetic construct
  REGION 1..20: note = Synthetic: FlagHis Tag
GSGDYKDDDDKGSGHHHHHH

SEQ ID NO: 261 - protein, 22 aa, synthetic construct
  REGION 1..22: note = Synthetic: MycHis
GSGEQKLISEEDLGSGHHHHHH

SEQ ID NO: 262 - protein, 497 aa, synthetic construct
  REGION 1..497: note = Synthetic: aEpCAM x hOKT3 (MT110/AMG110)
MELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 263 - protein, 509 aa, synthetic construct
  REGION 1..509: note = Synthetic: aEpCAM x 2037-B10
MELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSEVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVTHHHHHH

SEQ ID NO: 264 - protein, 249 aa, synthetic construct
  REGION 1..249: note = Synthetic: SRP2037:A01
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPDTPARFSGSLLGGKAALTLSGVQPEDEAEYYCALWDSTRWVFGGGTQLTVT

SEQ ID NO: 265 - protein, 249 aa, synthetic construct
  REGION 1..249: note = Synthetic: SRP2037:A02
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 266 - protein, 249 aa, synthetic construct
  REGION 1..249: note = Synthetic: SRP2037:A03
EVQLVESGGGLVKPGGSLRLSCAASGFTFSKYAMNWVRQAPGKGLEWVARIRSKYNNYATYYADSVKDRFTISRDDSKNTAYLQINSLKTEDTAVYYCVRHGNFGNSYTSYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 267 - protein, 249 aa, synthetic construct
  REGION 1..249: note = Synthetic: SRP2037:A04
EVQLVESGGGLVKPGGSLRLSCAASGFTFNKYAMNWVRQAPGKGLEWVARIRSKYNNYATYYADSVKDRFTISRDDSKSTAYLQMNSLKTEDTAVYYCARHGNFGNSYISYWAYWGQGTPVTVSSGGGGSGGDGSGGGGSQTVVTQEPSLTVSPGGTVTLTCGSSTGAVTSGYHPNWLQQKPGQAPRGLIGGTKFLAPGTPARFSGSLLGGKAALTLSGVQPEDEAEYYCALWCSNRWVFGGGTQLTVT

SEQ ID NO: 268 - protein, 249 aa, synthetic construct
  REGION 1..249: note = Synthetic: >SRP2037:A05
EVQLVESGGGLVKPGGSLRLSCAASGFTFNKYAMNWVRQAPGKGLEWVARIRSKYNNYATYYADSVKDRFTISRDDSRNTAYLQMNSLKAEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 269 - protein, 249 aa, synthetic construct
  REGION 1..249: note = Synthetic: SRP2037:A06
EVQLVESGGGLVKPGDSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 270 - protein, 249 aa, synthetic construct
  REGION 1..249: note = Synthetic: SRP2037:A07
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARLSGSLLGGKAALTLSGVQPEDEAEYYCALWYSNRWVFGGGTQLTVT

SEQ ID NO: 271 - protein, 249 aa, synthetic construct
  REGION 1..249: note = Synthetic: SRP2037:A08
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGSGTQLTVT

SEQ ID NO: 272 - protein, 249 aa, synthetic construct; identical to SEQ ID NO: 265
  REGION 1..249: note = Synthetic: SRP2037:A09

SEQ ID NO: 273 - protein, 249 aa, synthetic construct
  REGION 1..249: note = Synthetic: SRP2037:A11
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWFSNRWVFGSGTQLTVT

SEQ ID NO: 274 - protein, 249 aa, synthetic construct
  REGION 1..249: note = Synthetic: SRP2037:B01
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAAYHCALWYSNRWVFGGGTQLTVT

SEQ ID NO: 275 - protein, 249 aa, synthetic construct; identical to SEQ ID NO: 271
  REGION 1..249: note = Synthetic: SRP2037:B02

SEQ ID NO: 276 - protein, 249 aa, synthetic construct
  REGION 1..249: note = Synthetic: SRP2037:B03
EVQLVESGGGLVKPGGGLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 277 - protein, 249 aa, synthetic construct
  REGION 1..249: note = Synthetic: SRP2037:B04
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGCYPNWLQQKPGQAPRGLIGGTKFLAPGTPARFSGSLLGGKAALZLSGVQPEDEAEYYCALWYSNRWVFGGGTQLTVT

SEQ ID NO: 278 - protein, 249 aa, synthetic construct
  REGION 1..249: note = Synthetic: SRP2037:B05
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWDSNRWVFGGGTQLTVT

SEQ ID NO: 279 - protein, 249 aa, synthetic construct
  REGION 1..249: note = Synthetic: SRP2037:B06
EVQLVESGGGLVKPGGSLRLSCAASGFTFNKYAMNWVRQAPGKGLEWVARIRSKYNNYETYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGDGTQLTVT

SEQ ID NO: 280 - protein, 249 aa, synthetic construct
  REGION 1..249: note = Synthetic: SRP2037:B07
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVLSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 281 - protein, 249 aa, synthetic construct
  REGION 1..249: note = Synthetic: SRP2037:B08
EVQLVESGGGLVRPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCVLWYSNRWVFGGGTQLTVT

SEQ ID NO: 282 - protein, 249 aa, synthetic construct
  REGION 1..249: note = Synthetic: SRP2037:B09
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGTGGDGSGGGGSQTVVTQEPSLTVSPGGTVTLTCGSSTGAVTSGYYPNWLQQKPGQAPRGLIGGTKFLAPGTPARFSGSLLGGKAALTLSGVRPEDEAEYYCALWQSNRWZFGGGTQLTVT

SEQ ID NO: 283 - protein, 249 aa, synthetic construct
  REGION 1..249: note = Synthetic: SRP2037:B10
EVQLVESGGGLVKPGGSLRLSCAASGFTFNKYAMNWVRQAPGKGLEWVARIRTKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVT

SEQ ID NO: 284 - protein, 249 aa, synthetic construct; identical to SEQ ID NO: 265
  REGION 1..249: note = Synthetic: SRP2037:B11

SEQ ID NO: 285 - protein, 249 aa, synthetic construct
  REGION 1..249: note = Synthetic: SRP2037:C01
EVQLVESGGGLVKPGGSLRLSCVASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGSGTQLTVT

SEQ ID NO: 286 - protein, 748 aa, synthetic construct
  REGION 1..748: note = Synthetic: SRP2037:C02
GLUVALGLNLEUVALGLUSERGLYGLYGLYLEUVALLYSPROGLYGLYSERLEUARGLEUSERCYSALAALASERGLYPHETHRPHEASNLYSTYRALAMETASNTRPVALARGGLNALAPROGLYLYSGLYLEUGLUTRPVALALAARGILEARGSERLYSTYRASNASNTYRALATHRTYRTYRALAASPSERVALLYSASPARGPHETHRILESERARGASPASPSERLYSASNTHRALATYRLEUGLNMETASNSERLEULYSTHRGLUASPTHRALAVALTYRTYRCYSVALARGHISGLYASNPHEGLYASNSERTYRILESERTYRTRPALATYRTRPGLYGLNGLYTHRPROVALTHRVALSERSERGLYGLYGLYGLYSERGLYGLYASPGLYSERGLYGLYGLYGLYSERGLNTHRVALVALTHRGLNGLUPROSERLEUTHRVALSERPROGLYGLYTHRVALTHRLEUTHRCYSGLYSERSERTHRGLYALAVALTHRSERGLYTYRTYRPROASNTRPLEUGLNGLNLYSPROGLYGLNALAPROARGGLYLEUILEGLYGLYTHRLYSPHELEUALAPROGLYTHRPROALAARGPHESERGLYSERLEULEUGLYGLYLYSALAALALEUTHRLEUSERGLYVALGLNPROGLUASPDGLUALAGLXTYRTYRCYSALALEUTRPGLNSERASNARGTRPVALPHEGLYGLYGLYTHRGLNLEUTHRVALTHR

SEQ ID NO: 287 - protein, 249 aa, synthetic construct
  REGION 1..249: note = Synthetic: SRP2037:C03
EVQLVESGGGLVKPGGSLRLSCVASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGIKFLAPGTPARFSGSLLGGKAALTLSGVQPEDEAEYYCALWYSNRWVFGGGTQLTVT

SEQ ID NO: 288 - protein, 249 aa, synthetic construct
  REGION 1..249: note = Synthetic: SRP2037:C04
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWSATAGWFGGGTQLTVT

SEQ ID NO: 289 - protein, 249 aa, synthetic construct
  REGION 1..249: note = Synthetic: SRP2037:C05
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSTVGWFGGGTQLTVT

SEQ ID NO: 290 - protein, 249 aa, synthetic construct
  REGION 1..249: note = Synthetic: SRP2037:C06
EVQLVESGGGLVKPGGSLRLSCAASGFTFNKYAMNWVRQAPGKGLEWVARIRSKYNNYATYYADSVKDRFTISRDDSKNTAYLQMNSLKTEDTAVYYCVRHGNFGNSYISYWAYWGQGTPVTVSSGGGGSGGDGSGGGGSQTVVTQEPSLTVSPGGTVTLTCGSSTGAVTSGYYPNWLQQKPGQAPRGLIGGTKFLAPGTPARFSGSLLGGKAALZLSGVQPEDEAEYYCALWYSNRWVFGGGTQLTVT

SEQ ID NO: 291 - protein, 11 aa, synthetic construct
  REGION 1..11: note = Synthetic: exemplary scFv-Fc linker
AAGSDQEPKSS

SEQ ID NO: 292 - protein, 20 aa, synthetic construct
  REGION 1..20: note = Synthetic: C-terminal FlagHis tag
GSGDYKDDDDKGSGHHHHHH

SEQ ID NO: 293 - protein, 15 aa, synthetic construct
  REGION 1..15: note = Synthetic: Linker
GGGGSGGGGSGGGGS

SEQ ID NO: 294 - protein, 14 aa, synthetic construct
  REGION 1..14: note = Synthetic: consensus sequence
  VARIANT 3: note = X is N or D
  VARIANT 4: note = X is F or D
  VARIANT 5: note = X is G or D
  VARIANT 6: note = X is N or D
  VARIANT 7: note = X is S or D
  VARIANT 8: note = X is Y or D
HGXXXXXXISYWAY

SEQ ID NO: 295 - protein, 19 aa, synthetic construct
  REGION 1..19: note = Synthetic: consensus sequence
  VARIANT 1: note = X is R or D
  VARIANT 3: note = X is R or D
  VARIANT 4: note = X is S or T
  VARIANT 5: note = X is K or D
  VARIANT 6: note = X is Y or D
  VARIANT 7: note = X is N or D
  VARIANT 10: note = X is A or D
  VARIANT 12: note = X is Y or D
XIXXXXXNYXTXYADSVKD

SEQ ID NO: 296 - protein, 9 aa, synthetic construct
  REGION 1..9: note = Synthetic: consensus sequence
  VARIANT 3: note = X is W or D
  VARIANT 4: note = X is Y, F, or D
  VARIANT 5: note = X is S or D
  VARIANT 6: note = X is N or D
  VARIANT 7: note = X is R or D
ALXXXXXWV

SEQ ID NO: 297 - protein, 442 aa, synthetic construct
  REGION 1..442: note = Synthetic: Engmab_83A10_1-VL_CH1_Fc
MQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 298 - protein, 233 aa, synthetic construct
  REGION 1..233: note = Synthetic: aCD3-Engmab_83A10_3-VH_CL
MEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 7 aa, synthetic construct
  REGION 1..7: note = Synthetic: consensus sequence
GTKFLAP

SEQ ID NO: 300 - protein, 14 aa, synthetic construct
  REGION 1..14: note = Synthetic: consensus sequence
GSSTGAVTSGYYPN

SEQ ID NO: 301 - protein, 19 aa, synthetic construct
  REGION 1..19: note = Synthetic: consensus sequence
  VARIANT 1: note = X is R or D
  VARIANT 3: note = X is R or D
  VARIANT 4: note = X is S or T
  VARIANT 5: note = X is K or D
  VARIANT 6: note = X is Y or D
  VARIANT 7: note = X is N or D
  VARIANT 10: note = X is A or D
  VARIANT 12: note = X is Y or D
XIXXXXXNYXTXYADSVKD

SEQ ID NO: 302 - protein, 25 aa, synthetic construct
  REGION 1..25: note = Synthetic: consensus sequence
  VARIANT 5: note = X is L or V
  VARIANT 13: note = X is K or Q
  VARIANT 16: note = X is G or R
  VARIANT 19: note = X is K or R
  VARIANT 23: note = X is T or A
EVQLXESGGGLVXPGXSLXLSCXAS

SEQ ID NO: 303 - protein, 14 aa, synthetic construct
  REGION 1..14: note = Synthetic: consensus sequence
  VARIANT 2: note = X is V or F
  VARIANT 6: note = X is S or P
WXRQAXGKGLEWVA

SEQ ID NO: 304 - protein, 32 aa, synthetic construct
  REGION 1..32: note = Synthetic: consensus sequence
  VARIANT 11: note = X is S or N
  VARIANT 12: note = X is I or T
  VARIANT 19: note = X is S or N
  VARIANT 21: note = X is R or K
  VARIANT 22: note = X A or T
RFTISRDDSKXXAYLQMNXLXXEDTAVYYCVR

SEQ ID NO: 305 - protein, 11 aa, synthetic construct
  REGION 1..11: note = Synthetic: consensus sequence
  VARIANT 6: note = X is P or L
WGQGTXVTVSS

SEQ ID NO: 306 - protein, 15 aa, synthetic construct
  REGION 1..15: note = Synthetic: consensus sequence
  VARIANT 2: note = X is Y, F, V, or L
  VARIANT 5: note = X is T, K, or H
  VARIANT 8: note = X is Q or K
  VARIANT 11: note = X is R or K
  VARIANT 12: note = X is G or L
  VARIANT 13: note = X is L or M
WXQQXPGXAPXXXIG

SEQ ID NO: 307 - protein, 32 aa, synthetic construct
  REGION 1..32: note = Synthetic: consensus sequence
  VARIANT 2: note = X is V, T, or I
  VARIANT 4: note = X is D, A, or S
  VARIANT 10: note = X is L or G
  VARIANT 11: note = X is L or S
  VARIANT 13: note = X is T, G, or N
  VARIANT 14: note = X is K, T, or D
  VARIANT 15: note = X is A or F
  VARIANT 16: note = X is A, S, or T
  VARIANT 19: note = X is L or I
  VARIANT 20: note = X is T or S
  VARIANT 21: note = X is G, R, or S
  VARIANT 22: note = X is A, V, or L
  VARIANT 23: note = X is Q or E
  VARIANT 24: note = X is A or P
  VARIANT 25: note = X is D or E
  VARIANT 27: note = X is F or E
  VARIANT 28: note = X is S or A
  VARIANT 29: note = X is T, E, V, or D
GXPXRFSGSXXGXXXXLTXXXXXXXDXXXYYC

SEQ ID NO: 308 - protein, 5 aa, synthetic construct
  REGION 1..5: note = Synthetic: Linker
GGGGS

SEQ ID NO: 309 - protein, 30 aa, synthetic construct
  REGION 1..30: note = Synthetic: Linker
GGGGSGGGGSGGGGSGGGGSGGGGSGGGGS

(19) United States
(12) Patent Application Publication
(10) Pub. No.: US 2024/0124585 A1
(43) Pub. Date: Apr. 18, 2024

(54) ANTI-CD3 ANTIBODIES, COMPOSITIONS COMPRISING ANTI-CD3 ANTIBODIES AND METHODS OF MAKING AND USING ANTI-CD3 ANTIBODIES

(71) Applicant: SUTRO BIOPHARMA, INC., South San Francisco, CA (US)

(72) Inventors: Ryan STAFFORD, Emeryville, CA (US); Junhao YANG, Palo Alto, CA (US); Alice YAM, Tiburon, CA (US); Joni CASTRO, South San Francisco, CA (US); Gang YIN, South San Francisco, CA (US); Cuong TRAN, South San Francisco, CA (US); Xiaofan LI, Fremont, CA (US); Abigail YU, San Jose, CA (US)

(21) Appl. No.: 18/478,200

(22) Filed: Sep. 29, 2023

Related U.S. Application Data
(60) Provisional application No. 63/411,303, filed on Sep. 29, 2022.

(51) Int. Cl. C07K 16/28

(52) U.S. Cl. CPC C07K 16/2809; C07K 2317/565

(57) ABSTRACT

The present disclosure relates to antibodies that selectively bind to CD3 and its isoforms and homologs, and compositions comprising the antibodies. Also provided are methods of using the antibodies, such as therapeutic and diagnostic methods.

### CROSS-REFERENCE TO RELATED APPLICATION

This application claims priority to U.S. Provisional Patent Application No. 63/411,303, filed Sep. 29, 2022. The foregoing application is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 22, 2023, is named “108843.00467.xml” and is 427,124 bytes in size. The sequence listing contained in this .XML file is part of the specification and is hereby incorporated by reference herein in its entirety.

### FIELD OF THE INVENTION

The present disclosure generally relates to antibodies with binding specificity for cluster of differentiation 3 (CD3) and compositions comprising the antibodies, including pharmaceutical compositions, diagnostic compositions, and kits. Also provided are methods of making anti-CD3 antibodies, and methods of using anti-CD3 antibodies, for example, for therapeutic purposes, diagnostic purposes, and research purposes.

### BACKGROUND

The cellular immune system involves mobilizing T-cells that serve a variety of roles. T cells are immunologically specific and act against or in response to a variety of antigens. These antigens may come from foreign sources or from the host having been altered by neoplasia or infection. Although T cells do not themselves secrete antibodies, they are usually required for antibody secretion by the second class of lymphocytes.

T-cell activation requires a T cell receptor (TCR) to recognize a peptide in association with a CD3 complex. CD3 is a cell surface protein complex associated with the T-cell receptor. The association of CD3 with the TCR-peptide transmits an activation signal via intracellular signaling molecules, brought about by the CD3 protein complex undergoing a conformational change, to initiate a signaling cascade in the T cell. Smith-Garvin, et al., 2009, Annu Rev Immunology. 2009; 27: 591-619.

The CD3 protein complex comprises four chains: a CD3 gamma chain, a CD3 delta chain, and two CD3 epsilon chains. The CD3 epsilon subunit of the T cell receptor (TCR) complex contains two defined signaling domains, a proline-rich sequence, and an immune tyrosine activation motifs (ITAMs). Id.

There is a need for improved methods of targeting and/or modulating the activity of CD3. Given the specific expression and association of CD3 with TCRs, there is a need for improved therapeutics that can specifically target cells and tissues expressing CD3.

### SUMMARY

Provided herein are antibodies that selectively bind CD3. In some embodiments, the antibodies bind human CD3. In some embodiments, the antibodies also bind homologs of human CD3.

In some embodiments, the antibodies comprise, consist essentially of, or consist of, an illustrative CDR, framework, V_{H}, or V_{L}, or combination thereof, sequence provided in this disclosure, or a variant thereof. In some aspects, the variant is a variant with one or more conservative amino acid substitutions. In some aspects, the variant has sequence identity to the illustrative sequence or sequences.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-CD3 antibodies provided herein. In some embodiments, the method is a method of treatment. In some embodiments, the method is a diagnostic method. In some embodiments, the method is an analytical method. In some embodiments, the method is a method of purifying and/or quantifying CD3.

In some embodiments, the antibodies are used to treat a disease or condition. In some aspects, the disease or condition is selected from a cancer, autoimmune disease, and infection.

These and other embodiments of the invention along with many of its features are described in more detail in conjunction with the text below and attached figures.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.p-0oi9uygxdz

FIGS. 2-3 provide alignments of the VH sequences (SEQ ID NOs:206-231) for the antibodies provided herein. CDRs according to Chothia are boxed, and CDRs according to Kabat are shaded.

FIGS. 4-5 provides alignments of the VL sequences (SEQ ID NOs:232-255) from the antibodies provided herein. CDRs according to Chothia and Kabat are highlighted.

FIG. 6A shows the results of a re-directed T cell killing assay NCI-H1975 in Donor #1. FIG. 6B shows the results of a re-directed T cell killing assay NCI-H1975 in Donor #2. FIG. 6C shows the results of a re-directed T cell killing CHO-k in Donor #1. FIG. 6D shows the results of a re-directed T cell killing CHO-k in Donor #2.

FIG. 7 shows an SDS-PAGE analysis of Phytip captured anti-CD3 IgG. NR is nonreducing; R is reducing.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to. For example, a sentence stating that “if α₂ is A, then α₃ is not D; α₅ is not S; or α₆ is not S; or combinations thereof” includes the following combinations when α₂ is A: (1) α₃ is not D; (2) α₅ is not S; (3) α₆ is not S; (4) α₃ is not D; α₅ is not S; and α₆ is not S; (5) α₃ is not D and α₅ is not S; (6) α₃ is not D and α₆ is not S; and (7) α₅ is not S and α₆ is not S. In another example, if a set of antibody heavy chains comprise {A, B, and C} and a set of antibody light chains comprise {D, E, and F} then the potential combinations of heavy and light chains comprise the set of {AD, AE, AF, BD, BE, BF, CD, CE, and CF}.

The term “CD3” refers to, unless specified otherwise, any variants, isoforms and species homologs of human cluster of differentiation 3 (CD3) that are naturally expressed by cells, or that are expressed by cells transfected with a CD3 or CD3 gene. CD3 proteins include, for example, human CD3 (SEQ ID NO: 1). In some embodiments, CD3 proteins include cynomolgus monkey CD3 (SEQ ID NO: 2). In some embodiments, CD3 proteins include murine CD3 (SEQ ID NO: 3).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “CD3 antibody,” “anti-CD3 antibody,” “CD3 Ab,” “CD3-specific antibody,” “anti-CD3 Ab,” “CD3 antibody,” “anti-CD3 antibody,” “CD3 Ab,” “CD3-specific antibody,” or “anti-CD3 Ab” is an antibody, as described herein, which binds specifically to CD3 or CD3. In some embodiments, the antibody binds the extracellular domain of CD3. As used herein, unless otherwise specified, an antibody may also comprise an “antibody fragment” as defined herewithin.

The V_{H} and V_{L} regions may be further subdivided into regions of hypervariability (“hypervariable regions (HVRs)” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J Mol. Biol., 2001, 309:657-70 (“AHo” numbering scheme), each of which is incorporated by reference in its entirety.

Table 1A provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes. Table 1B provides the positions of LFR1, CDR-L1, LFR2, CDR-L2, LFR3, CDR-L3, LFR4, HFR1, CDR-H1, HFR2, CDR-H2, HFR3, CDR-H3, and HFR4 as identified by the Kabat and Chothia schemes.

TABLE 1A
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 | H31-H35 or H35B | H26-H32 or H34* |
| H2 | H50-H65 | H52-H56 |
| H3 | H95-H102 | H95-H102 |

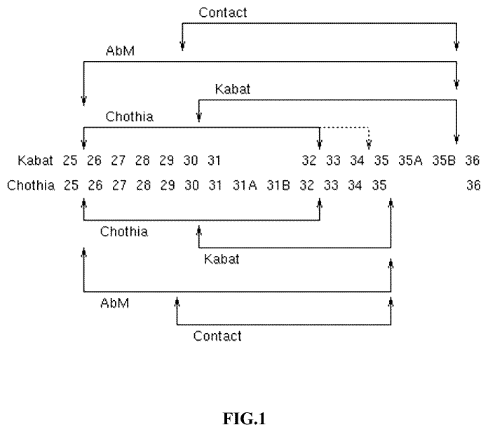

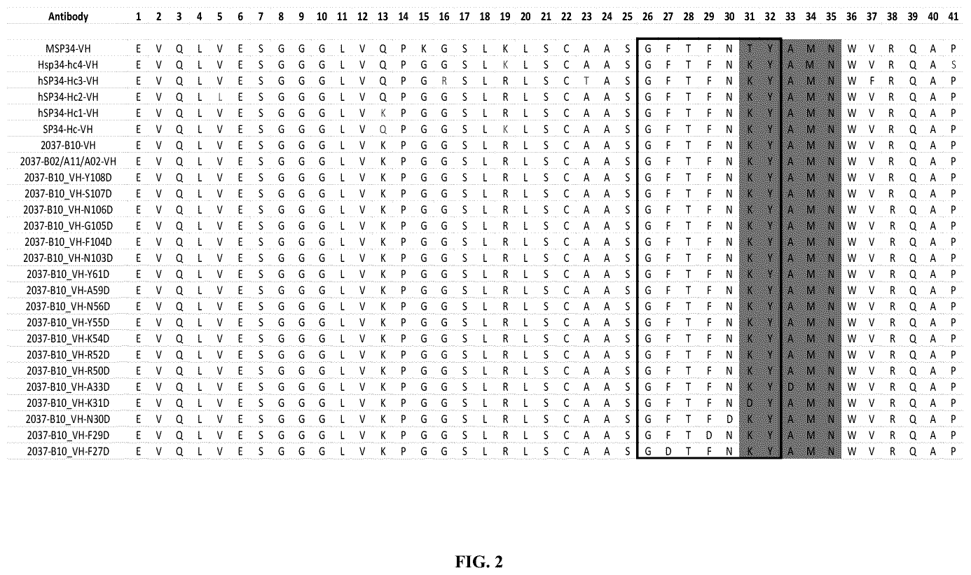

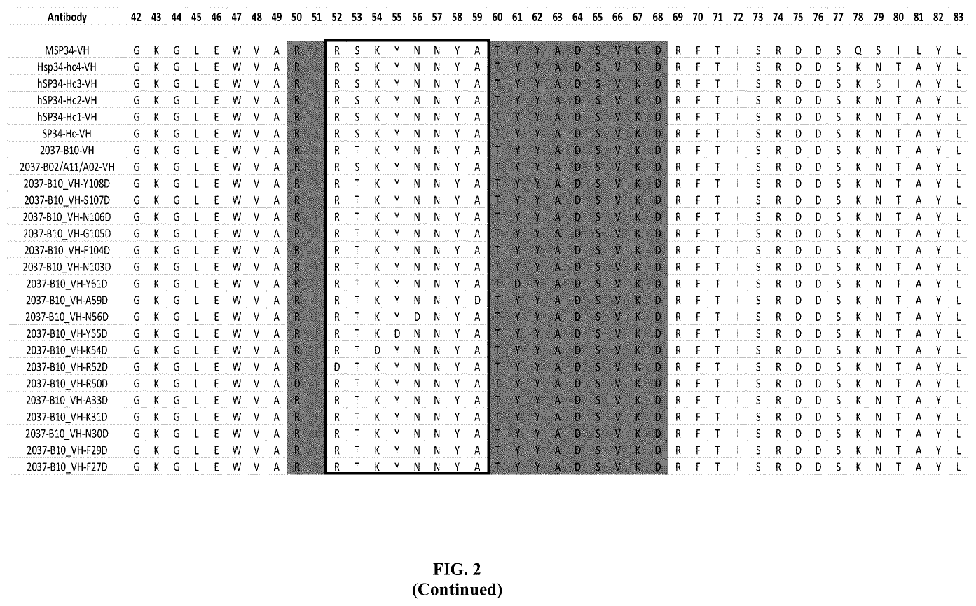

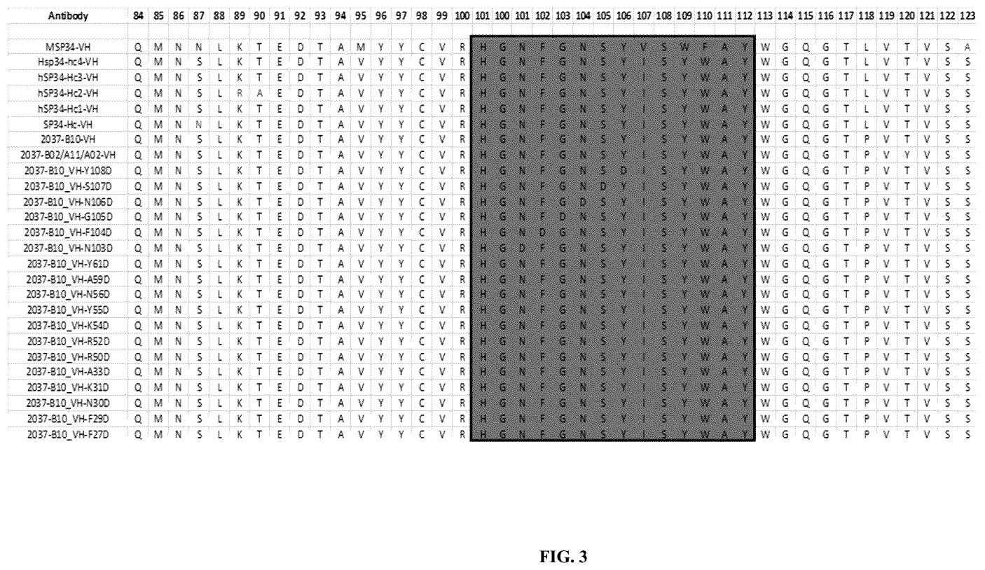

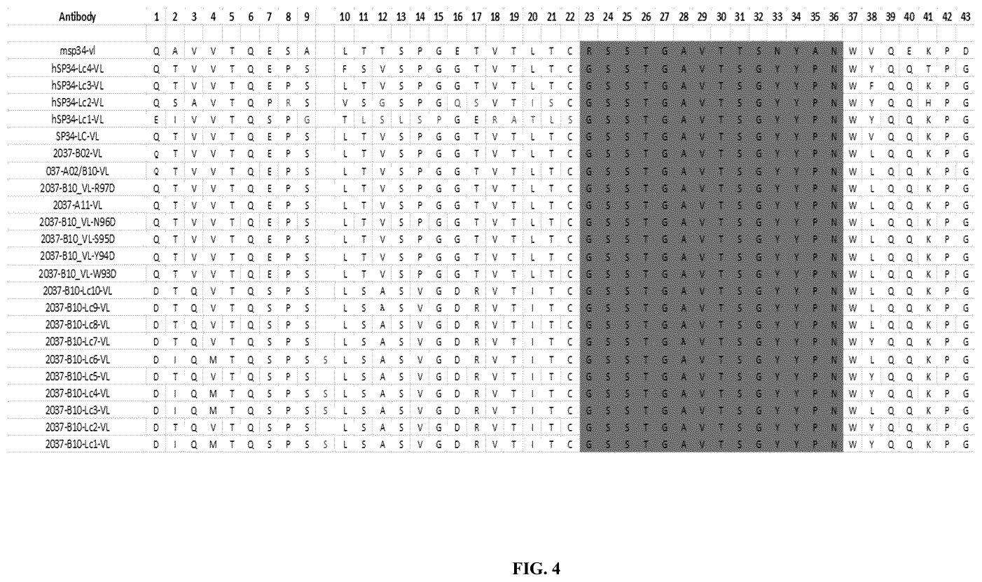

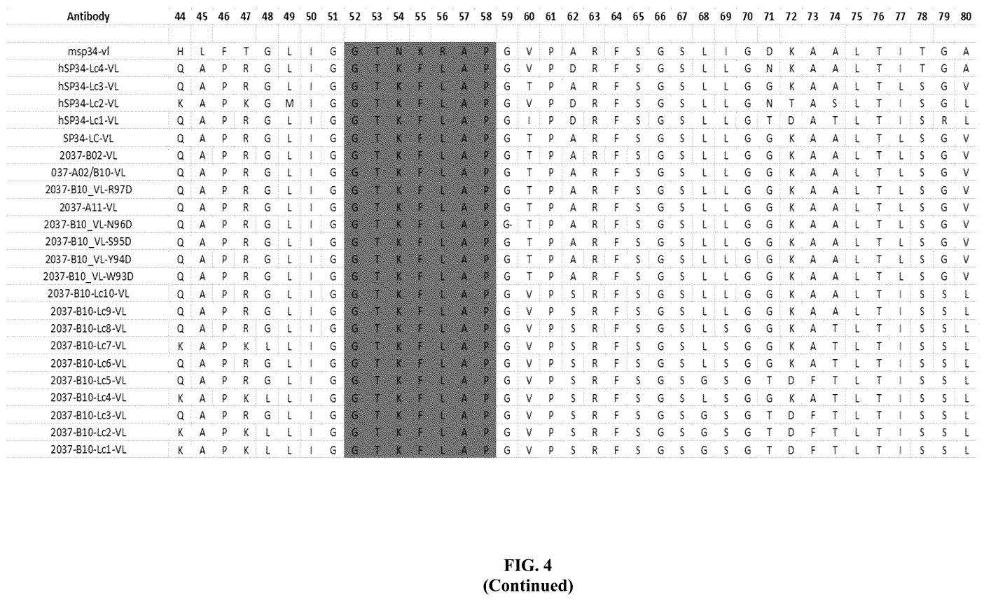

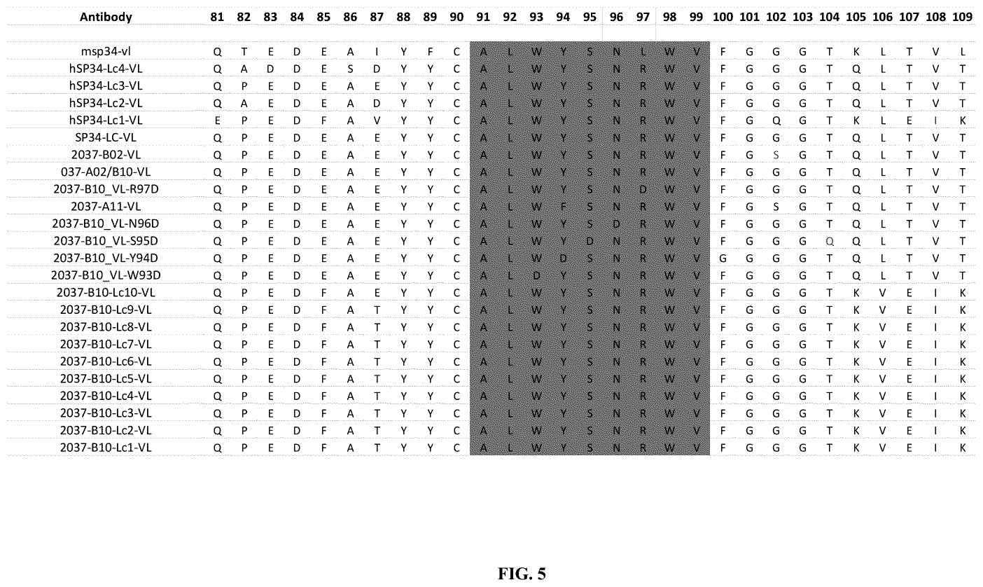

*The C-terminus of CDR-H1, when numbered using the Kabat numbering convention, varies between H32 and H34, depending on the length of the CDR, as illustrated in FIG. 1.

TABLE 1B
Residues in CDRs according to Kabat
and Chothia numbering schemes.

| CDR | Kabat | Chothia |
|---|---|---|
| LFR1 | L1-23 | L1-23 |
| L1 | L24-L34 | L24-L34 |
| LFR2 | L35-49 | L35-49 |
| L2 | L50-L56 | L50-L56 |
| LFR3 | L57-88 | L57-88 |
| L3 | L89-L97 | L89-L97 |
| LFR4 | L98-107 | L98-107 |
| HFR1 | H1-30 | H1-25 |
| H1 | H31-H35 or H35B | H26-H32 or H34* |
| HFR2 | H36-49 | H33-51 |
| H2 | H50-H65 | H52-H56 |
| HFR3 | H66-94 | H57-94 |
| H3 | H95-H102 | H95-H102 |
| HFR4 | H103-113 | H103-113 |

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinf.org.uk/abs/abnum, and described in Abhinandan and Martin, Immunology, 2008, 45:3832-3839, incorporated by reference in its entirety.

The “framework region” refers to a subdivision of the variable region acting as a scaffold for the CDRs. Unless specified otherwise, there are four framework regions for each heavy chain and light chain, wherein each CDRs is flanked by one framework region. Unless stated otherwise, the numbering of the framework region is based on using either the Kabat or Chothia numbering scheme. Table 1B provides the positions of framework positions LFR1, LFR2, LFR3, LFR4, HFR1, HFR2, HFR3, and HFR4 as identified by the Kabat and Chothia schemes.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a VH domain and a V_{L} domain in a single polypeptide chain. The V_{H} and V_{L} are generally linked by a peptide linker. See Plückthun A. (1994). In some embodiments, the linker is SEQ ID NO: 591. In some embodiments, the linker is SEQ ID NO: 592. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 584, or a portion thereof. SEQ ID NO: 584 provides the sequence of C_{H1}, C_{H2}, and C_{H3} of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐk_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., CD3). In one exemplary assay, CD3 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of CD3 with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, R, T, V, |
| | W, and Y |
| Negatively Charged Residues | D and E |
| Polar Residues | C, D, E, H, K, N, Q, R, S, and T |
| Positively Charged Residues | H, K, and R |
| Small Residues | A, C, D, G, N, P, S, T, and V |
| Very Small Residues | A, G, and S |
| Residues Involved in Turn Formation | A, C, D, E, G, H, K, N, Q, R, S, |
| | P, and T |
| Flexible Residues | Q, T, K, S, G, P, D, E, and R |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a disease that can be treated or diagnosed with an antibody provided herein. In some embodiments, the disease is gastric carcinoma, colorectal carcinoma, renal cell carcinoma, cervical carcinoma, non-small cell lung carcinoma, ovarian cancer, prostate cancer, and/or a cancer of epithelial origin.

### 2. Antibodies

Provided herein are antibodies that selectively bind CD3. In some aspects, the antibodies selectively bind to human CD3. In some aspects, the antibodies selectively bind to the extracellular domain of human CD3 (human CD3).

In some embodiments, the antibody binds to a homolog of human CD3. In some aspects, the antibody binds to a homolog of human CD3 from a species selected from monkeys, mice, dogs, cats, rats, cows, horses, goats and sheep. In some aspects, the homolog is a cynomolgus monkey homolog. In some aspects, the homolog is a mouse or murine analog.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 7, 8, 9, 10, 11, 12, 13, 14, 15, or 16 residues in length.

In some aspects, the Kabat/Chothia CDR-L1 of the antibody is 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

### Heavy Chain Sequences

### 2.1. CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs: 207-231.

In some embodiments, the antibody comprises a CDR-H3 sequence comprising, consisting of, or consisting essentially of a sequence selected from SEQ ID NOs: 109-133. In some aspects, the antibody comprises a CDR-H3 sequence comprising, consisting of, or consisting essentially of SEQ ID NO: 109. In some aspects, the antibody comprises a CDR-H3 sequence comprising, consisting of, or consisting essentially of SEQ ID NO: 110. In some aspects, the antibody comprises a CDR-H3 sequence comprising, consisting of, or consisting essentially of SEQ ID NO: 111. In some aspects, the antibody comprises a CDR-H3 sequence comprising, consisting of, or consisting essentially of SEQ ID NO: 112. In some aspects, the antibody comprises a CDR-H3 sequence comprising, consisting of, or consisting essentially of SEQ ID NO: 113. In some aspects, the antibody comprises a CDR-H3 sequence comprising, consisting of, or consisting essentially of SEQ ID NO: 114. In some aspects, the antibody comprises a CDR-H3 sequence comprising, consisting of, or consisting essentially of SEQ ID NO: 115. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 2.2. V_{H} Sequences Comprising Illustrative CDRs

In some embodiments, the antibody comprises a V_{H} sequence comprising one or more CDR-H3 sequences comprising, consisting of, or consisting essentially of one or more illustrative CDR-H3 sequences provided in this disclosure, and variants thereof. In some embodiments, the CDR-H3 sequences comprise, consist of, or consist essentially of one or more CDR-H3 sequences provided in a V_{H} sequence selected from SEQ ID NOs: 207-231.

### 2.2.1. V_{H} Sequences Comprising Illustrative Kabat CDRs

In some embodiments, the antibody comprises a V_{H} sequence comprising one or more Kabat CDR-H3 sequences comprising, consisting of, or consisting essentially of one or more illustrative Kabat CDR-H3 sequences provided in this disclosure, and variants thereof.

### 2.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 207-231.

In some embodiments, the antibody comprises a V_{H} sequence comprising a Kabat CDR-H3 sequence comprising, consisting of, or consisting essentially of a sequence selected from SEQ ID NOs: 109-133. In some aspects, the antibody comprises a CDR-H3 sequence comprising, consisting of, or consisting essentially of SEQ ID NO: 109. In some aspects, the antibody comprises a CDR-H3 sequence comprising, consisting of, or consisting essentially of SEQ ID NO: 110. In some aspects, the antibody comprises a CDR-H3 sequence comprising, consisting of, or consisting essentially of SEQ ID NO: 111. In some aspects, the antibody comprises a CDR-H3 sequence comprising, consisting of, or consisting essentially of SEQ ID NO: 112. In some aspects, the antibody comprises a CDR-H3 sequence comprising, consisting of, or consisting essentially of SEQ ID NO: 113. In some aspects, the antibody comprises a CDR-H3 sequence comprising, consisting of, or consisting essentially of SEQ ID NO: 114. In some aspects, the antibody comprises a CDR-H3 sequence comprising, consisting of, or consisting essentially of SEQ ID NO: 115. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133.

### 2.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 207-231.

In some embodiments, the antibody comprises a V_{H} sequence comprising a Kabat CDR-H2 sequence comprising, consisting of, or consisting essentially of a sequence selected from SEQ ID NOs: 83-107. In some aspects, the antibody comprises a CDR-H2 sequence comprising, consisting of, or consisting essentially of SEQ ID NO: 83. In some aspects, the antibody comprises a CDR-H2 sequence comprising, consisting of, or consisting essentially of SEQ ID NO: 84. In some aspects, the antibody comprises a CDR-H2 sequence comprising, consisting of, or consisting essentially of SEQ ID NO: 85. In some aspects, the antibody comprises a CDR-H2 sequence comprising, consisting of, or consisting essentially of SEQ ID NO: 86. In some aspects, the antibody comprises a CDR-H2 sequence comprising, consisting of, or consisting essentially of SEQ ID NO: 87. In some aspects, the antibody comprises a CDR-H2 sequence comprising, consisting of, or consisting essentially of SEQ ID NO: 88. In some aspects, the antibody comprises a CDR-H2 sequence comprising, consisting of, or consisting essentially of SEQ ID NO: 89. In some aspects, the antibody comprises a CDR-H2 sequence comprising, consisting of, or consisting essentially of SEQ ID NO: 90. In some aspects, the antibody comprises a CDR-H2 sequence comprising, consisting of, or consisting essentially of SEQ ID NO: 91. In some aspects, the antibody comprises a CDR-H2 sequence comprising, consisting of, or consisting essentially of SEQ ID NO: 92. In some aspects, the antibody comprises a CDR-H2 sequence comprising, consisting of, or consisting essentially of SEQ ID NO: 93. In some aspects, the antibody comprises a CDR-H2 sequence comprising, consisting of, or consisting essentially of SEQ ID NO: 94. In some aspects, the antibody comprises a CDR-H2 sequence comprising, consisting of, or consisting essentially of SEQ ID NO: 95. In some aspects, the antibody comprises a CDR-H2 sequence comprising, consisting of, or consisting essentially of SEQ ID NO: 96. In some aspects, the antibody comprises a CDR-H2 sequence comprising, consisting of, or consisting essentially of SEQ ID NO: 97. In some aspects, the antibody comprises a CDR-H2 sequence comprising, consisting of, or consisting essentially of SEQ ID NO: 98. In some aspects, the antibody comprises a CDR-H2 sequence comprising, consisting of, or consisting essentially of SEQ ID NO: 99. In some aspects, the antibody comprises a CDR-H2 sequence comprising, consisting of, or consisting essentially of SEQ ID NO: 100. In some aspects, the antibody comprises a CDR-H2 sequence comprising, consisting of, or consisting essentially of SEQ ID NO: 101. In some aspects, the antibody comprises a CDR-H2 sequence comprising, consisting of, or consisting essentially of SEQ ID NO: 102. In some aspects, the antibody comprises a CDR-H2 sequence comprising, consisting of, or consisting essentially of SEQ ID NO: 103. In some aspects, the antibody comprises a CDR-H2 sequence comprising, consisting of, or consisting essentially of SEQ ID NO: 104. In some aspects, the antibody comprises a CDR-H2 sequence comprising, consisting of, or consisting essentially of SEQ ID NO: 105. In some aspects, the antibody comprises a CDR-H2 sequence comprising, consisting of, or consisting essentially of SEQ ID NO: 106. In some aspects, the antibody comprises a CDR-H2 sequence comprising, consisting of, or consisting essentially of SEQ ID NO: 107.

### 2.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 207-231.

In some embodiments, the antibody comprises a V_{H} sequence comprising a Kabat CDR-H1 sequence comprising, consisting of, or consisting essentially of a sequence selected from SEQ ID NOs: 31-55. In some aspects, the antibody comprises a CDR-H1 sequence comprising, consisting of, or consisting essentially of SEQ ID NO: 31. In some aspects, the antibody comprises a CDR-H1 sequence comprising, consisting of, or consisting essentially of SEQ ID NO: 32. In some aspects, the antibody comprises a CDR-H1 sequence comprising, consisting of, or consisting essentially of SEQ ID NO: 33. In some aspects, the antibody comprises a CDR-H1 sequence comprising, consisting of, or consisting essentially of SEQ ID NO: 34. In some aspects, the antibody comprises a CDR-H1 sequence comprising, consisting of, or consisting essentially of SEQ ID NO: 35. In some aspects, the antibody comprises a CDR-H1 sequence comprising, consisting of, or consisting essentially of SEQ ID NO: 36. In some aspects, the antibody comprises a CDR-H1 sequence comprising, consisting of, or consisting essentially of SEQ ID NO: 37. In some aspects, the antibody comprises a CDR-H1 sequence comprising, consisting of, or consisting essentially of SEQ ID NO: 38. In some aspects, the antibody comprises a CDR-H1 sequence comprising, consisting of, or consisting essentially of SEQ ID NO: 39. In some aspects, the antibody comprises a CDR-H1 sequence comprising, consisting of, or consisting essentially of SEQ ID NO: 40. In some aspects, the antibody comprises a CDR-H1 sequence comprising, consisting of, or consisting essentially of SEQ ID NO: 41. In some aspects, the antibody comprises a CDR-H1 sequence comprising, consisting of, or consisting essentially of SEQ ID NO: 42. In some aspects, the antibody comprises a CDR-H1 sequence comprising, consisting of, or consisting essentially of SEQ ID NO: 43. In some aspects, the antibody comprises a CDR-H1 sequence comprising, consisting of, or consisting essentially of SEQ ID NO: 44. In some aspects, the antibody comprises a CDR-H1 sequence comprising, consisting of, or consisting essentially of SEQ ID NO: 45. In some aspects, the antibody comprises a CDR-H1 sequence comprising, consisting of, or consisting essentially of SEQ ID NO: 45. In some aspects, the antibody comprises a CDR-H1 sequence comprising, consisting of, or consisting essentially of SEQ ID NO: 46. In some aspects, the antibody comprises a CDR-H1 sequence comprising, consisting of, or consisting essentially of SEQ ID NO: 47. In some aspects, the antibody comprises a CDR-H1 sequence comprising, consisting of, or consisting essentially of SEQ ID NO: 48. In some aspects, the antibody comprises a CDR-H1 sequence comprising, consisting of, or consisting essentially of SEQ ID NO: 49. In some aspects, the antibody comprises a CDR-H1 sequence comprising, consisting of, or consisting essentially of SEQ ID NO: 50. In some aspects, the antibody comprises a CDR-H1 sequence comprising, consisting of, or consisting essentially of SEQ ID NO: 51. In some aspects, the antibody comprises a CDR-H1 sequence comprising, consisting of, or consisting essentially of SEQ ID NO: 52. In some aspects, the antibody comprises a CDR-H1 sequence comprising, consisting of, or consisting essentially of SEQ ID NO: 53. In some aspects, the antibody comprises a CDR-H1 sequence comprising, consisting of, or consisting essentially of SEQ ID NO: 54. In some aspects, the antibody comprises a CDR-H1 sequence comprising, consisting of, or consisting essentially of SEQ ID NO: 55.

### 2.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 109-133, and a Kabat CDR-H2 sequence comprising, consisting of, or consisting essentially of a sequence selected from SEQ ID NOs: 83-107. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 207-231.

### 2.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 109-133, and a Kabat CDR-H1 sequence comprising, consisting of, or consisting essentially of a sequence selected from SEQ ID NOs: 31-35. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 207-231.

### 2.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 31-55 and a Kabat CDR-H2 sequence comprising, consisting of, or consisting essentially of a sequence selected from SEQ ID NOs: 83-107. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 207-231.

### 2.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 31-55, a Kabat CDR-H2 sequence comprising, consisting of, or consisting essentially of a sequence selected from SEQ ID NOs: 83-107, and a Kabat CDR-H3 sequence comprising, consisting of, or consisting essentially of a sequence selected from SEQ ID NOs: 109-133. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 207-231.

In some embodiments, the antibody comprises three Kabat HC CDRs selected from the group consisting of SEQ ID NOs: 35/87/113; 34/86/112; 33/85/111; 32/84/110; 31/83/109; 37/89/115; 36/88/114; 38/64/90; 39/91/117; 40/92/118; 41/93/119; 42/94/120; 44/96/122; 45/97/123; 46/98/124; 47/99/125; 48/100/126; 49/101/127; 50/102/128; 51/103/129; 52/104/130; 53/105/131; 54/106/132; 55/107/133; and 43/95/121.

### 2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, 95%, 96%, 97%, 98%, or 99% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, 95%, 96%, 97%, 98%, or 99% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, 95%, 96%, 97%, 98%, or 99% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 2.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 2.2.2.1. Chothia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 207-231.

In some embodiments, the antibody comprises a V_{H} sequence comprising a Chothia CDR-H3 sequence comprising, consisting of, or consisting essentially of a sequence selected from SEQ ID NOs: 109-133. In some aspects, the antibody comprises a CDR-H3 sequence comprising, consisting of, or consisting essentially of SEQ ID NO: 109. In some aspects, the antibody comprises a CDR-H3 sequence comprising, consisting of, or consisting essentially of SEQ ID NO: 110. In some aspects, the antibody comprises a CDR-H3 sequence comprising, consisting of, or consisting essentially of SEQ ID NO: 111. In some aspects, the antibody comprises a CDR-H3 sequence comprising, consisting of, or consisting essentially of SEQ ID NO: 112. In some aspects, the antibody comprises a CDR-H3 sequence comprising, consisting of, or consisting essentially of SEQ ID NO: 113. In some aspects, the antibody comprises a CDR-H3 sequence comprising, consisting of, or consisting essentially of SEQ ID NO: 114. In some aspects, the antibody comprises a CDR-H3 sequence comprising, consisting of, or consisting essentially of SEQ ID NO: 115. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some aspects, the antibody comprises a CDR-H3 sequence comprising, consisting of, or consisting essentially of SEQ ID NO: 132. In some aspects, the antibody comprises a CDR-H3 sequence comprising, consisting of, or consisting essentially of SEQ ID NO: 133.

### 2.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 207-231.

In some embodiments, the antibody comprises a V_{H} sequence comprising a Chothia CDR-H2 sequence comprising, consisting of, or consisting essentially of a sequence selected from SEQ ID NOs: 57-81. In some aspects, the antibody comprises a CDR-H2 sequence comprising, consisting of, or consisting essentially of SEQ ID NO: 57. In some aspects, the antibody comprises a CDR-H2 sequence comprising, consisting of, or consisting essentially of SEQ ID NO: 58. In some aspects, the antibody comprises a CDR-H2 sequence comprising, consisting of, or consisting essentially of SEQ ID NO: 59. In some aspects, the antibody comprises a CDR-H2 sequence comprising, consisting of, or consisting essentially of SEQ ID NO: 60. In some aspects, the antibody comprises a CDR-H2 sequence comprising, consisting of, or consisting essentially of SEQ ID NO: 61.

In some aspects, the antibody comprises a CDR-H2 sequence comprising, consisting of, or consisting essentially of SEQ ID NO: 62. In some aspects, the antibody comprises a CDR-H2 sequence comprising, consisting of, or consisting essentially of SEQ ID NO: 63. In some aspects, the antibody comprises a CDR-H2 sequence comprising, consisting of, or consisting essentially of SEQ ID NO: 64. In some aspects, the antibody comprises a CDR-H2 sequence comprising, consisting of, or consisting essentially of SEQ ID NO: 65. In some aspects, the antibody comprises a CDR-H2 sequence comprising, consisting of, or consisting essentially of SEQ ID NO: 66. In some aspects, the antibody comprises a CDR-H2 sequence comprising, consisting of, or consisting essentially of SEQ ID NO: 67. In some aspects, the antibody comprises a CDR-H2 sequence comprising, consisting of, or consisting essentially of SEQ ID NO: 68. In some aspects, the antibody comprises a CDR-H2 sequence comprising, consisting of, or consisting essentially of SEQ ID NO: 69. In some aspects, the antibody comprises a CDR-H2 sequence comprising, consisting of, or consisting essentially of SEQ ID NO: 70. In some aspects, the antibody comprises a CDR-H2 sequence comprising, consisting of, or consisting essentially of SEQ ID NO: 71. In some aspects, the antibody comprises a CDR-H2 sequence comprising, consisting of, or consisting essentially of SEQ ID NO: 72. In some aspects, the antibody comprises a CDR-H2 sequence comprising, consisting of, or consisting essentially of SEQ ID NO: 73. In some aspects, the antibody comprises a CDR-H2 sequence comprising, consisting of, or consisting essentially of SEQ ID NO: 74. In some aspects, the antibody comprises a CDR-H2 sequence comprising, consisting of, or consisting essentially of SEQ ID NO: 75. In some aspects, the antibody comprises a CDR-H2 sequence comprising, consisting of, or consisting essentially of SEQ ID NO: 76. In some aspects, the antibody comprises a CDR-H2 sequence comprising, consisting of, or consisting essentially of SEQ ID NO: 77. In some aspects, the antibody comprises a CDR-H2 sequence comprising, consisting of, or consisting essentially of SEQ ID NO: 78. In some aspects, the antibody comprises a CDR-H2 sequence comprising, consisting of, or consisting essentially of SEQ ID NO: 79. In some aspects, the antibody comprises a CDR-H2 sequence comprising, consisting of, or consisting essentially of SEQ ID NO: 80. In some aspects, the antibody comprises a CDR-H2 sequence comprising, consisting of, or consisting essentially of SEQ ID NO: 81.

### 2.2.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 207-231.

In some embodiments, the antibody comprises a V_{H} sequence comprising a Chothia CDR-H1 sequence comprising, consisting of, or consisting essentially of a sequence selected from SEQ ID NOs: 5-29. In some aspects, the antibody comprises a CDR-H1 sequence comprising, consisting of, or consisting essentially of SEQ ID NO: 5. In some aspects, the antibody comprises a CDR-H1 sequence comprising, consisting of, or consisting essentially of SEQ ID NO: 6. In some aspects, the antibody comprises a CDR-H1 sequence comprising, consisting of, or consisting essentially of SEQ ID NO: 7. In some aspects, the antibody comprises a CDR-H1 sequence comprising, consisting of, or consisting essentially of SEQ ID NO: 8. In some aspects, the antibody comprises a CDR-H1 sequence comprising, consisting of, or consisting essentially of SEQ ID NO: 9. In some aspects, the antibody comprises a CDR-H1 sequence comprising, consisting of, or consisting essentially of SEQ ID NO: 10. In some aspects, the antibody comprises a CDR-H1 sequence comprising, consisting of, or consisting essentially of SEQ ID NO: 11. In some aspects, the antibody comprises a CDR-H1 sequence comprising, consisting of, or consisting essentially of SEQ ID NO: 12. In some aspects, the antibody comprises a CDR-H1 sequence comprising, consisting of, or consisting essentially of SEQ ID NO: 13. In some aspects, the antibody comprises a CDR-H1 sequence comprising, consisting of, or consisting essentially of SEQ ID NO: 14. In some aspects, the antibody comprises a CDR-H1 sequence comprising, consisting of, or consisting essentially of SEQ ID NO: 15. In some aspects, the antibody comprises a CDR-H1 sequence comprising, consisting of, or consisting essentially of SEQ ID NO: 16. In some aspects, the antibody comprises a CDR-H1 sequence comprising, consisting of, or consisting essentially of SEQ ID NO: 17. In some aspects, the antibody comprises a CDR-H1 sequence comprising, consisting of, or consisting essentially of SEQ ID NO: 18. In some aspects, the antibody comprises a CDR-H1 sequence comprising, consisting of, or consisting essentially of SEQ ID NO: 19. In some aspects, the antibody comprises a CDR-H1 sequence comprising, consisting of, or consisting essentially of SEQ ID NO: 20. In some aspects, the antibody comprises a CDR-H1 sequence comprising, consisting of, or consisting essentially of SEQ ID NO: 21. In some aspects, the antibody comprises a CDR-H1 sequence comprising, consisting of, or consisting essentially of SEQ ID NO: 22. In some aspects, the antibody comprises a CDR-H1 sequence comprising, consisting of, or consisting essentially of SEQ ID NO: 23. In some aspects, the antibody comprises a CDR-H1 sequence comprising, consisting of, or consisting essentially of SEQ ID NO: 24. In some aspects, the antibody comprises a CDR-H1 sequence comprising, consisting of, or consisting essentially of SEQ ID NO: 25. In some aspects, the antibody comprises a CDR-H1 sequence comprising, consisting of, or consisting essentially of SEQ ID NO: 26. In some aspects, the antibody comprises a CDR-H1 sequence comprising, consisting of, or consisting essentially of SEQ ID NO: 27. In some aspects, the antibody comprises a CDR-H1 sequence comprising, consisting of, or consisting essentially of SEQ ID NO: 28. In some aspects, the antibody comprises a CDR-H1 sequence comprising, consisting of, or consisting essentially of SEQ ID NO: 29.

### 2.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs:109-133, and a Chothia CDR-H2 sequence comprising, consisting of, or consisting essentially of a sequence selected from SEQ ID NOs:57-81. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs:207-231.

### 2.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 109-133, and a Chothia CDR-H1 sequence comprising, consisting of, or consisting essentially of a sequence selected from SEQ ID NOs: 5-29. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 207-231.

### 2.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 5-29 and a Chothia CDR-H2 sequence comprising, consisting of, or consisting essentially of a sequence selected from SEQ ID NOs: 57-81. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 207-231.

### 2.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 5-29, a Chothia CDR-H2 sequence comprising, consisting of, or consisting essentially of a sequence selected from SEQ ID NOs: 57-81, and a Chothia CDR-H3 sequence comprising, consisting of, or consisting essentially of a sequence selected from SEQ ID NOs: 109-133. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 207-231.

In some embodiments, the isolated antibody comprises three Chothia HC CDRs selected from the group consisting of SEQ ID NOs: 9/61/113; 8/60/112; 7/59/111; 6/58/110; 5/57/109; 11/63/115; 10/62/114; 12/64/116; 13/65/117; 14/66/118; 15/67/119; 16/68/120; 18/70/122; 19/71/123; 20/72/124; 21/73/125; 22/74/126; 23/75/127; 24/76/128; 25/77/129; 26/78/130; 27/79/131; 28/80/132; and 29/81/133.

### 2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, 95%, 96%, 97%, 98%, or 99% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, 95%, 96%, 97%, 98%, or 99% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, 95%, 96%, 97%, 98%, or 99% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 2.3. V_{H} Sequences

In some embodiments, the antibody comprises, consists of, or consists essentially of a V_{H} sequence provided in SEQ ID NOs: 207-231.

In some embodiments, the antibody comprises a CDR-H3, a CDR-H2, and a CDR-H1, each consisting of a CDR sequence of a V_{H} having the sequence set forth in one of SEQ ID NOS:207-231. In some embodiments, the antibody comprises a CDR-H3, a CDR-H2, a CDR-H1, and framework regions, each consisting of a CDR sequence or framework sequence of a V_{H} having the sequence set forth in one of SEQ ID NOS:207-231.

In some embodiments, the antibody comprises a V_{H} sequence comprising, consisting of, or consisting essentially of a sequence selected from SEQ ID NOs: 207-231. In some aspects, the antibody comprises a V_{H} sequence comprising, consisting of, or consisting essentially of SEQ ID NO: 207. In some aspects, the antibody comprises a V_{H} sequence comprising, consisting of, or consisting essentially of SEQ ID NO: 208. In some aspects, the antibody comprises a V_{H} sequence comprising, consisting of, or consisting essentially of SEQ ID NO: 209. In some aspects, the antibody comprises a V_{H} sequence comprising, consisting of, or consisting essentially of SEQ ID NO: 210. In some aspects, the antibody comprises a V_{H} sequence comprising, consisting of, or consisting essentially of SEQ ID NO: 411. In some aspects, the antibody comprises a V_{H} sequence comprising, consisting of, or consisting essentially of SEQ ID NO: 412. In some aspects, the antibody comprises a V_{H} sequence comprising, consisting of, or consisting essentially of SEQ ID NO: 413. In some aspects, the antibody comprises a V_{H} sequence comprising, consisting of, or consisting essentially of SEQ ID NO: 414. In some aspects, the antibody comprises a V_{H} sequence comprising, consisting of, or consisting essentially of SEQ ID NO: 415. In some aspects, the antibody comprises a V_{H} sequence comprising, consisting of, or consisting essentially of SEQ ID NO: 416. In some aspects, the antibody comprises a V_{H} sequence comprising, consisting of, or consisting essentially of SEQ ID NO: 417. In some aspects, the antibody comprises a V_{H} sequence comprising, consisting of, or consisting essentially of SEQ ID NO: 418. In some aspects, the antibody comprises a V_{H} sequence comprising, consisting of, or consisting essentially of SEQ ID NO: 419. In some aspects, the antibody comprises a V_{H} sequence comprising, consisting of, or consisting essentially of SEQ ID NO: 420. In some aspects, the antibody comprises a V_{H} sequence comprising, consisting of, or consisting essentially of SEQ ID NO: 421. In some aspects, the antibody comprises a V_{H} sequence comprising, consisting of, or consisting essentially of SEQ ID NO: 422. In some aspects, the antibody comprises a V_{H} sequence comprising, consisting of, or consisting essentially of SEQ ID NO: 423. In some aspects, the antibody comprises a V_{H} sequence comprising, consisting of, or consisting essentially of SEQ ID NO: 424. In some aspects, the antibody comprises a V_{H} sequence comprising, consisting of, or consisting essentially of SEQ ID NO: 425. In some aspects, the antibody comprises a V_{H} sequence comprising, consisting of, or consisting essentially of SEQ ID NO: 426. In some aspects, the antibody comprises a V_{H} sequence comprising, consisting of, or consisting essentially of SEQ ID NO: 427. In some aspects, the antibody comprises a V_{H} sequence comprising, consisting of, or consisting essentially of SEQ ID NO: 428. In some aspects, the antibody comprises a V_{H} sequence comprising, consisting of, or consisting essentially of SEQ ID NO: 429. In some aspects, the antibody comprises a V_{H} sequence comprising, consisting of, or consisting essentially of SEQ ID NO: 430. In some aspects, the antibody comprises a V_{H} sequence comprising, consisting of, or consisting essentially of SEQ ID NO: 431.

### 2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### Light Chain Sequences

### 2.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 2.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 233-255.

In some embodiments, the antibody comprises a V_{L} sequence comprising a CDR-L3 sequence comprising, consisting of, or consisting essentially of a sequence selected from SEQ ID NOs: 183-205. In some aspects, the antibody comprises a CDR-L3 sequence comprising, consisting of, or consisting essentially of SEQ ID NO: 183. In some aspects, the antibody comprises a CDR-L3 sequence comprising, consisting of, or consisting essentially of SEQ ID NO: 184. In some aspects, the antibody comprises a CDR-L3 sequence comprising, consisting of, or consisting essentially of SEQ ID NO: 185. In some aspects, the antibody comprises a CDR-L3 sequence comprising, consisting of, or consisting essentially of SEQ ID NO: 186. In some aspects, the antibody comprises a CDR-L3 sequence comprising, consisting of, or consisting essentially of SEQ ID NO: 187. In some aspects, the antibody comprises a CDR-L3 sequence comprising, consisting of, or consisting essentially of SEQ ID NO: 188. In some aspects, the antibody comprises a CDR-L3 sequence comprising, consisting of, or consisting essentially of SEQ ID NO: 189. In some aspects, the antibody comprises a CDR-L3 sequence comprising, consisting of, or consisting essentially of SEQ ID NO: 190. In some aspects, the antibody comprises a CDR-L3 sequence comprising, consisting of, or consisting essentially of SEQ ID NO: 191. In some aspects, the antibody comprises a CDR-L3 sequence comprising, consisting of, or consisting essentially of SEQ ID NO: 192. In some aspects, the antibody comprises a CDR-L3 sequence comprising, consisting of, or consisting essentially of SEQ ID NO: 193. In some aspects, the antibody comprises a CDR-L3 sequence comprising, consisting of, or consisting essentially of SEQ ID NO: 194. In some aspects, the antibody comprises a CDR-L3 sequence comprising, consisting of, or consisting essentially of SEQ ID NO: 195. In some aspects, the antibody comprises a CDR-L3 sequence comprising, consisting of, or consisting essentially of SEQ ID NO: 196. In some aspects, the antibody comprises a CDR-L3 sequence comprising, consisting of, or consisting essentially of SEQ ID NO: 197. In some aspects, the antibody comprises a CDR-L3 sequence comprising, consisting of, or consisting essentially of SEQ ID NO: 198. In some aspects, the antibody comprises a CDR-L3 sequence comprising, consisting of, or consisting essentially of SEQ ID NO: 199. In some aspects, the antibody comprises a CDR-L3 sequence comprising, consisting of, or consisting essentially of SEQ ID NO: 200. In some aspects, the antibody comprises a CDR-L3 sequence comprising, consisting of, or consisting essentially of SEQ ID NO: 201. In some aspects, the antibody comprises a CDR-L3 sequence comprising, consisting of, or consisting essentially of SEQ ID NO: 202. In some aspects, the antibody comprises a CDR-L3 sequence comprising, consisting of, or consisting essentially of SEQ ID NO: 203. In some aspects, the antibody comprises a CDR-L3 sequence comprising, consisting of, or consisting essentially of SEQ ID NO: 204. In some aspects, the antibody comprises a CDR-L3 sequence comprising, consisting of, or consisting essentially of SEQ ID NO: 205.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs: 233-255.

In some embodiments, the antibody comprises a V_{L} sequence comprising a CDR-L2 sequence comprising, consisting of, or consisting essentially of a sequence selected from SEQ ID NOs: 159-181. In some aspects, the antibody comprises a V_{L} sequence comprising a CDR-L2 sequence comprising, consisting of, or consisting essentially of SEQ ID NO: 159. In some aspects, the antibody comprises a V_{L} sequence comprising a CDR-L2 sequence comprising, consisting of, or consisting essentially of SEQ ID NO: 160. In some aspects, the antibody comprises a V_{L} sequence comprising a CDR-L2 sequence comprising, consisting of, or consisting essentially of SEQ ID NO: 161. In some aspects, the antibody comprises a V_{L} sequence comprising a CDR-L2 sequence comprising, consisting of, or consisting essentially of SEQ ID NO: 162. In some aspects, the antibody comprises a CDR-L2 sequence comprising, consisting of, or consisting essentially of SEQ ID NO: 163. In some aspects, the antibody comprises a CDR-L2 sequence comprising, consisting of, or consisting essentially of SEQ ID NO: 164. In some aspects, the antibody comprises a CDR-L2 sequence comprising, consisting of, or consisting essentially of SEQ ID NO: 165. In some aspects, the antibody comprises a CDR-L2 sequence comprising, consisting of, or consisting essentially of SEQ ID NO: 166. In some aspects, the antibody comprises a CDR-L2 sequence comprising, consisting of, or consisting essentially of SEQ ID NO: 167. In some aspects, the antibody comprises a CDR-L2 sequence comprising, consisting of, or consisting essentially of SEQ ID NO: 168. In some aspects, the antibody comprises a CDR-L2 sequence comprising, consisting of, or consisting essentially of SEQ ID NO: 169. In some aspects, the antibody comprises a CDR-L2 sequence comprising, consisting of, or consisting essentially of SEQ ID NO: 170. In some aspects, the antibody comprises a CDR-L2 sequence comprising, consisting of, or consisting essentially of SEQ ID NO: 171. In some aspects, the antibody comprises a CDR-L2 sequence comprising, consisting of, or consisting essentially of SEQ ID NO: 172. In some aspects, the antibody comprises a CDR-L2 sequence comprising, consisting of, or consisting essentially of SEQ ID NO: 174. In some aspects, the antibody comprises a CDR-L2 sequence comprising, consisting of, or consisting essentially of SEQ ID NO: 175. In some aspects, the antibody comprises a CDR-L2 sequence comprising, consisting of, or consisting essentially of SEQ ID NO: 176. In some aspects, the antibody comprises a CDR-L2 sequence comprising, consisting of, or consisting essentially of SEQ ID NO: 17. In some aspects, the antibody comprises a CDR-L2 sequence comprising, consisting of, or consisting essentially of SEQ ID NO: 178. In some aspects, the antibody comprises a CDR-L2 sequence comprising, consisting of, or consisting essentially of SEQ ID NO: 179. In some aspects, the antibody comprises a CDR-L2 sequence comprising, consisting of, or consisting essentially of SEQ ID NO: 180. In some aspects, the antibody comprises a CDR-L2 sequence comprising, consisting of, or consisting essentially of SEQ ID NO: 181.

### 2.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs: 233-255.

In some embodiments, the antibody comprises a V_{L} sequence comprising a CDR-L1 sequence comprising, consisting of, or consisting essentially of a sequence selected from SEQ ID NOs: 135-157. In some aspects, the antibody comprises a V_{L} sequence comprising a CDR-L1 sequence comprising, consisting of, or consisting essentially of SEQ ID NO: 135. In some aspects, the antibody comprises a V_{L} sequence comprising a CDR-L1 sequence comprising, consisting of, or consisting essentially of SEQ ID NO: 136. In some aspects, the antibody comprises a V_{L} sequence comprising a CDR-L1 sequence comprising, consisting of, or consisting essentially of SEQ ID NO: 137. In some aspects, the antibody comprises a V_{L} sequence comprising a CDR-L1 sequence comprising, consisting of, or consisting essentially of SEQ ID NO: 138. In some aspects, the antibody comprises a V_{L} sequence comprising a CDR-L1 sequence comprising, consisting of, or consisting essentially of SEQ ID NO: 139. In some aspects, the antibody comprises a V_{L} sequence comprising a CDR-L1 sequence comprising, consisting of, or consisting essentially of SEQ ID NO: 140. In some aspects, the antibody comprises a V_{L} sequence comprising a CDR-L1 sequence comprising, consisting of, or consisting essentially of SEQ ID NO: 141. In some aspects, the antibody comprises a V_{L} sequence comprising a CDR-L1 sequence comprising, consisting of, or consisting essentially of SEQ ID NO: 142. In some aspects, the antibody comprises a V_{L} sequence comprising a CDR-L1 sequence comprising, consisting of, or consisting essentially of SEQ ID NO: 143. In some aspects, the antibody comprises a V_{L} sequence comprising a CDR-L1 sequence comprising, consisting of, or consisting essentially of SEQ ID NO: 144. In some aspects, the antibody comprises a V_{L} sequence comprising a CDR-L1 sequence comprising, consisting of, or consisting essentially of SEQ ID NO: 145. In some aspects, the antibody comprises a V_{L} sequence comprising a CDR-L1 sequence comprising, consisting of, or consisting essentially of SEQ ID NO: 146. In some aspects, the antibody comprises a V_{L} sequence comprising a CDR-L1 sequence comprising, consisting of, or consisting essentially of SEQ ID NO: 147. In some aspects, the antibody comprises a V_{L} sequence comprising a CDR-L1 sequence comprising, consisting of, or consisting essentially of SEQ ID NO: 148. In some aspects, the antibody comprises a V_{L} sequence comprising a CDR-L1 sequence comprising, consisting of, or consisting essentially of SEQ ID NO: 149. In some aspects, the antibody comprises a V_{L} sequence comprising a CDR-L1 sequence comprising, consisting of, or consisting essentially of SEQ ID NO: 150. In some aspects, the antibody comprises a V_{L} sequence comprising a CDR-L1 sequence comprising, consisting of, or consisting essentially of SEQ ID NO: 151. In some aspects, the antibody comprises a V_{L} sequence comprising a CDR-L1 sequence comprising, consisting of, or consisting essentially of SEQ ID NO: 152. In some aspects, the antibody comprises a V_{L} sequence comprising a CDR-L1 sequence comprising, consisting of, or consisting essentially of SEQ ID NO: 153. In some aspects, the antibody comprises a V_{L} sequence comprising a CDR-L1 sequence comprising, consisting of, or consisting essentially of SEQ ID NO: 154. In some aspects, the antibody comprises a V_{L} sequence comprising a CDR-L1 sequence comprising, consisting of, or consisting essentially of SEQ ID NO: 156. In some aspects, the antibody comprises a V_{L} sequence comprising a CDR-L1 sequence comprising, consisting of, or consisting essentially of SEQ ID NO: 157.

### 2.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 183-205 and a CDR-L2 sequence comprising, consisting of, or consisting essentially of a sequence selected from SEQ ID NOs: 159-181. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 233-255.

### 2.5.5. CDR-L3+CDR-L1

In some embodiments, the antibody comprises a V_{L} sequence comprising a CDR-L3 sequence comprising, consisting of, or consisting essentially of a sequence selected from SEQ ID NOs: 183-205 and a CDR-L1 sequence comprising, consisting of, or consisting essentially of a sequence selected from SEQ ID NOs: 135-157. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 233-255.

### 2.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 135-157 and a CDR-L2 sequence comprising, consisting of, or consisting essentially of a sequence selected from SEQ ID NOs: 159-181. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 233-255.

### 2.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 135-157, a CDR-L2 sequence comprising, consisting of, or consisting essentially of a sequence selected from SEQ ID NOs: 159-181, and a CDR-L3 sequence comprising, consisting of, or consisting essentially of a sequence selected from SEQ ID NOs: 183-205. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 233-255.

### 2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.6. V_{L} Sequences

In some embodiments, the antibody comprises, consists of, or consists essentially of a V_{L} sequence provided in SEQ ID NOs: 233-255.

In some embodiments, the antibody comprises a CDR-L3, a CDR-L2, and a CDR-L1, each consisting of a CDR sequence of a V_{L} having the sequence set forth in one of SEQ ID NOS:233-255. In some embodiments, the antibody comprises a CDR-L3, a CDR-L2, a CDR-L1, and framework regions, each consisting of a CDR sequence or framework sequence of a V_{L} having the sequence set forth in one of SEQ ID NOS:233-255.

In some embodiments, the antibody comprises a V_{L} sequence comprising, consisting of, or consisting essentially of a sequence selected from SEQ ID NOs: 233-255. In some aspects, the antibody comprises a V_{L} sequence comprising, consisting of, or consisting essentially of SEQ ID NO: 233. In some aspects, the antibody comprises a V_{L} sequence comprising, consisting of, or consisting essentially of SEQ ID NO: 234. In some aspects, the antibody comprises a V_{L} sequence comprising, consisting of, or consisting essentially of SEQ ID NO: 235. In some aspects, the antibody comprises a V_{L} sequence comprising, consisting of, or consisting essentially of SEQ ID NO: 236. In some aspects, the antibody comprises a V_{L} sequence comprising, consisting of, or consisting essentially of SEQ ID NO: 237. In some aspects, the antibody comprises a V_{L} sequence comprising, consisting of, or consisting essentially of SEQ ID NO: 238. In some aspects, the antibody comprises a V_{L} sequence comprising, consisting of, or consisting essentially of SEQ ID NO: 239. In some aspects, the antibody comprises a V_{L} sequence comprising, consisting of, or consisting essentially of SEQ ID NO: 240. In some aspects, the antibody comprises a V_{L} sequence comprising, consisting of, or consisting essentially of SEQ ID NO: 241. In some aspects, the antibody comprises a V_{L} sequence comprising, consisting of, or consisting essentially of SEQ ID NO: 242. In some aspects, the antibody comprises a V_{L} sequence comprising, consisting of, or consisting essentially of SEQ ID NO: 243. In some aspects, the antibody comprises a V_{L} sequence comprising, consisting of, or consisting essentially of SEQ ID NO: 244. In some aspects, the antibody comprises a V_{L} sequence comprising, consisting of, or consisting essentially of SEQ ID NO: 245. In some aspects, the antibody comprises a V_{L} sequence comprising, consisting of, or consisting essentially of SEQ ID NO: 246. In some aspects, the antibody comprises a V_{L} sequence comprising, consisting of, or consisting essentially of SEQ ID NO: 247. In some aspects, the antibody comprises a V_{L} sequence comprising, consisting of, or consisting essentially of SEQ ID NO: 248. In some aspects, the antibody comprises a V_{L} sequence comprising, consisting of, or consisting essentially of SEQ ID NO: 249. In some aspects, the antibody comprises a V_{L} sequence comprising, consisting of, or consisting essentially of SEQ ID NO: 250. In some aspects, the antibody comprises a V_{L} sequence comprising, consisting of, or consisting essentially of SEQ ID NO: 251. In some aspects, the antibody comprises a V_{L} sequence comprising, consisting of, or consisting essentially of SEQ ID NO: 252. In some aspects, the antibody comprises a V_{L} sequence comprising, consisting of, or consisting essentially of SEQ ID NO: 253. In some aspects, the antibody comprises a V_{L} sequence comprising, consisting of, or consisting essentially of SEQ ID NO: 254. In some aspects, the antibody comprises a V_{L} sequence comprising, consisting of, or consisting essentially of SEQ ID NO: 255.

### 2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7. Pairs

### 2.7.1. CDR-H3-CDR-L3 Pairs

In some embodiments, the antibody comprises a CDR-H3 sequence and a CDR-L3 sequence. In some embodiments, the CDR-H3 sequence is part of a V_{H} and the CDR-L3 sequence is part of a V_{L}. In some embodiments, the CDR-H3 sequence and the CDR-L3 sequence are from V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some embodiments, the CDR-H3 sequence is a CDR-H3 sequence comprising, consisting of, or consisting essentially of any one of SEQ ID NOs: 109-133, and the CDR-L3 sequence is a CDR-L3 sequence comprising, consisting of, or consisting essentially of any one of SEQ ID NOs: 183-205.

In some aspects, the CDR-H3-CDR-L3 pairs are selected from SEQ ID NOs: SEQ ID NOs: 113 and 187; 113 and 186; 113 and 185; 113 and 184; 113 and 183; 112 and 187; 112 and 186; 112 and 185; 112 and 184; 112 and 183; 111 and 187; 111 and 186; 111 and 185; 111 and 184; 111 and 183; 110 and 187; 110 and 186; 110 and 185; 110 and 184; 110 and 183; 109 and 187; 109 and 186; 109 and 185; 109 and 184; 109 and 183; 115 and 190; 115 and 189; 115 and 188; 114 and 190; 116 and 190; 117 and 190; 118 and 190; 119 and 190; 120 and 190; 122 and 190; 123 and 190; 124 and 190; 125 and 190; 126 and 190; 127 and 190; 128 and 190; 129 and 190; 130 and 190; 131 and 190; 132 and 190; 133 and 190; 114 and 191; 114 and 192; 114 and 193; 114 and 194; 114 and 195; 110 and 205; 110 and 204; 110 and 203; 110 and 202; 110 and 201; 110 and 200; 110 and 199; 110 and 198; 110 and 197; 110 and 196; 121 and 191; 118 and 191; 116 and 191; 113 and 191; 128 and 191; 121 and 191; 118 and 191; 116 and 191; 113 and 191; 128 and 191; 121 and 201; 118 and 201; 116 and 201; 113 and 201; 128 and 201; 122 and 200; 118 and 200; 116 and 200; 113 and 200; and 128 and 200.

### 2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, 95%, 96%, 97%, 98%, or 99% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.7.2. CDR-H1-CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}. In some embodiments, the CDR-H1 sequence and the CDR-L1 sequence are from V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some aspects, the CDR-H1 sequence is a Chothia CDR-H1 sequence comprising, consisting of, or consisting essentially of any one of SEQ ID NOs: 5-29, and the CDR-L1 sequence is a CDR-L1 sequence comprising, consisting of, or consisting essentially of any one of SEQ ID NOs: 135-157.

In some aspects, the CDR-H1 sequence is a Kabat CDR-H1 sequence comprising, consisting of, or consisting essentially of any one of SEQ ID NOs: 31-55, and the CDR-L1 sequence is a CDR-L1 sequence comprising, consisting of, or consisting essentially of any one of SEQ ID NOs: 135-157.

### 2.7.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, 95%, 96%, 97%, 98%, or 99% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 2.7.3. CDR-H2-CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}. In some embodiments, the CDR-H2 sequence and the CDR-L2 sequence are from V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some aspects, the CDR-H2 sequence is a Chothia CDR-H2 sequence comprising, consisting of, or consisting essentially of any one of SEQ ID NOs: 57-81, and the CDR-L2 sequence is a CDR-L2 sequence comprising, consisting of, or consisting essentially of any one of SEQ ID NOs: 159-181.

In some aspects, the CDR-H2 sequence is a Kabat CDR-H2 sequence comprising, consisting of, or consisting essentially of any one of SEQ ID NOs: 83-107, and the CDR-L2 sequence is a CDR-L2 sequence comprising, consisting of, or consisting essentially of any one of SEQ ID NOs: 159-181.

### 2.7.3.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, 95%, 96%, 97%, 98%, or 99% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

2.7.4. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of any one of SEQ ID NOs: 207-231, and the V_{L} sequence is a V_{L} sequence comprising, consisting of, or consisting essentially of any one of SEQ ID NOs: 233-255.

In some aspects, the V_{H}-V_{L} pairs are selected from SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some aspects, the V_{H}-V_{L} pairs are selected from SEQ ID NOs: 206 and 232; 207 and 232; 208 and 232; 209 and 210; 211 and 232; 212 and 232; 213 and 232; 214 and 232; 215 and 232; 216 and 232; 217 and 232; 218 and 232; 219 and 232; 220 and 232; 221 and 232; 222 and 232; 223 and 232; 224 and 232; 225 and 232; 226 and 232; 227 and 232; 228 and 232; 229 and 232; 230 and 232; 231 and 232.

In some aspects, the V_{H}-V_{L} pairs are selected from SEQ ID NOs: 206 and 233; 207 and 233; 208 and 233; 209 and 210; 211 and 233; 212 and 233; 213 and 233; 214 and 233; 215 and 233; 216 and 233; 217 and 233; 218 and 233; 219 and 233; 220 and 233; 221 and 233; 222 and 233; 223 and 233; 224 and 233; 225 and 233; 226 and 233; 227 and 233; 228 and 233; 229 and 233; 230 and 233; 231 and 233.

In some aspects, the V_{H}-V_{L} pairs are selected from SEQ ID NOs: 206 and 234; 207 and 234; 208 and 234; 209 and 210; 211 and 234; 212 and 234; 213 and 234; 214 and 234; 215 and 234; 216 and 234; 217 and 234; 218 and 234; 219 and 234; 220 and 234; 221 and 234; 222 and 234; 223 and 234; 224 and 234; 225 and 234; 226 and 234; 227 and 234; 228 and 234; 229 and 234; 230 and 234; 231 and 234.

In some aspects, the V_{H}-V_{L} pairs are selected from SEQ ID NOs: 206 and 235; 207 and 235; 208 and 235; 209 and 210; 211 and 235; 212 and 235; 213 and 235; 214 and 235; 215 and 235; 216 and 235; 217 and 235; 218 and 235; 219 and 235; 220 and 235; 221 and 235; 222 and 235; 223 and 235; 224 and 235; 225 and 235; 226 and 235; 227 and 235; 228 and 235; 229 and 235; 230 and 235; 231 and 235.

In some aspects, the V_{H}-V_{L} pairs are selected from SEQ ID NOs: 206 and 236; 207 and 236; 208 and 236; 209 and 210; 211 and 236; 212 and 236; 213 and 236; 214 and 236; 215 and 236; 216 and 236; 217 and 236; 218 and 236; 219 and 236; 220 and 236; 221 and 236; 222 and 236; 223 and 236; 224 and 236; 225 and 236; 226 and 236; 227 and 236; 228 and 236; 229 and 236; 230 and 236; 231 and 236.

In some aspects, the V_{H}-V_{L} pairs are selected from SEQ ID NOs: 206 and 237; 207 and 237; 208 and 237; 209 and 210; 211 and 237; 212 and 237; 213 and 237; 214 and 237; 215 and 237; 216 and 237; 217 and 237; 218 and 237; 219 and 237; 220 and 237; 221 and 237; 222 and 237; 223 and 237; 224 and 237; 225 and 237; 226 and 237; 227 and 237; 228 and 237; 229 and 237; 230 and 237; 231 and 237.

In some aspects, the V_{H}-V_{L} pairs are selected from SEQ ID NOs: 206 and 238; 207 and 238; 208 and 238; 209 and 210; 211 and 238; 212 and 238; 213 and 238; 214 and 238; 215 and 238; 216 and 238; 217 and 238; 218 and 238; 219 and 238; 220 and 238; 221 and 238; 222 and 238; 223 and 238; 224 and 238; 225 and 238; 226 and 238; 227 and 238; 228 and 238; 229 and 238; 230 and 238; 231 and 238.

In some aspects, the V_{H}-V_{L} pairs are selected from SEQ ID NOs: 206 and 239; 207 and 239; 208 and 239; 209 and 210; 211 and 239; 212 and 239; 213 and 239; 214 and 239; 215 and 239; 216 and 239; 217 and 239; 218 and 239; 219 and 239; 220 and 239; 221 and 239; 222 and 239; 223 and 239; 224 and 239; 225 and 239; 226 and 239; 227 and 239; 228 and 239; 229 and 239; 230 and 239; 231 and 239.

In some aspects, the V_{H}-V_{L} pairs are selected from SEQ ID NOs: 206 and 240; 207 and 240; 208 and 240; 209 and 210; 211 and 240; 212 and 240; 213 and 240; 214 and 240; 215 and 240; 216 and 240; 217 and 240; 218 and 240; 219 and 240; 220 and 240; 221 and 240; 222 and 240; 223 and 240; 224 and 240; 225 and 240; 226 and 240; 227 and 240; 228 and 240; 229 and 240; 230 and 240; 231 and 240.

In some aspects, the V_{H}-V_{L} pairs are selected from SEQ ID NOs: 206 and 241; 207 and 241; 208 and 241; 209 and 210; 211 and 241; 212 and 241; 213 and 241; 214 and 241; 215 and 241; 216 and 241; 217 and 241; 218 and 241; 219 and 241; 220 and 241; 221 and 241; 222 and 241; 223 and 241; 224 and 241; 225 and 241; 226 and 241; 227 and 241; 228 and 241; 229 and 241; 230 and 241; 231 and 241.

In some aspects, the V_{H}-V_{L} pairs are selected from SEQ ID NOs: 206 and 242; 207 and 242; 208 and 242; 209 and 210; 211 and 242; 212 and 242; 213 and 242; 214 and 242; 215 and 242; 216 and 242; 217 and 242; 218 and 242; 219 and 242; 220 and 242; 221 and 242; 222 and 242; 223 and 242; 224 and 242; 225 and 242; 226 and 242; 227 and 242; 228 and 242; 229 and 242; 230 and 242; 231 and 242.

In some aspects, the V_{H}-V_{L} pairs are selected from SEQ ID NOs: 206 and 243; 207 and 243; 208 and 243; 209 and 210; 211 and 243; 212 and 243; 213 and 243; 214 and 243; 215 and 243; 216 and 243; 217 and 243; 218 and 243; 219 and 243; 220 and 243; 221 and 243; 222 and 243; 223 and 243; 224 and 243; 225 and 243; 226 and 243; 227 and 243; 228 and 243; 229 and 243; 230 and 243; 231 and 243.

In some aspects, the V_{H}-V_{L} pairs are selected from SEQ ID NOs: 206 and 244; 207 and 244; 208 and 244; 209 and 210; 211 and 244; 212 and 244; 213 and 244; 214 and 244; 215 and 244; 216 and 244; 217 and 244; 218 and 244; 219 and 244; 220 and 244; 221 and 244; 222 and 244; 223 and 244; 224 and 244; 225 and 244; 226 and 244; 227 and 244; 228 and 244; 229 and 244; 230 and 244; 231 and 244.

In some aspects, the V_{H}-V_{L} pairs are selected from SEQ ID NOs: 206 and 245; 207 and 245; 208 and 245; 209 and 210; 211 and 245; 212 and 245; 213 and 245; 214 and 245; 215 and 245; 216 and 245; 217 and 245; 218 and 245; 219 and 245; 220 and 245; 221 and 245; 222 and 245; 223 and 245; 224 and 245; 225 and 245; 226 and 245; 227 and 245; 228 and 245; 229 and 245; 230 and 245; 231 and 245.

In some aspects, the V_{H}-V_{L} pairs are selected from SEQ ID NOs: 206 and 246; 207 and 246; 208 and 246; 209 and 210; 211 and 246; 212 and 246; 213 and 246; 214 and 246; 215 and 246; 216 and 246; 217 and 246; 218 and 246; 219 and 246; 220 and 246; 221 and 246; 222 and 246; 223 and 246; 224 and 246; 225 and 246; 226 and 246; 227 and 246; 228 and 246; 229 and 246; 230 and 246; 231 and 246.

In some aspects, the V_{H}-V_{L} pairs are selected from SEQ ID NOs: 206 and 247; 207 and 247; 208 and 247; 209 and 210; 211 and 247; 212 and 247; 213 and 247; 214 and 247; 215 and 247; 216 and 247; 217 and 247; 218 and 247; 219 and 247; 220 and 247; 221 and 247; 222 and 247; 223 and 247; 224 and 247; 225 and 247; 226 and 247; 227 and 247; 228 and 247; 229 and 247; 230 and 247; 231 and 247.

In some aspects, the V_{H}-V_{L} pairs are selected from SEQ ID NOs: 206 and 248; 207 and 248; 208 and 248; 209 and 210; 211 and 248; 212 and 248; 213 and 248; 214 and 248; 215 and 248; 216 and 248; 217 and 248; 218 and 248; 219 and 248; 220 and 248; 221 and 248; 222 and 248; 223 and 248; 224 and 248; 225 and 248; 226 and 248; 227 and 248; 228 and 248; 229 and 248; 230 and 248; 231 and 248.

In some aspects, the V_{H}-V_{L} pairs are selected from SEQ ID NOs: 206 and 249; 207 and 249; 208 and 249; 209 and 210; 211 and 249; 212 and 249; 213 and 249; 214 and 249; 215 and 249; 216 and 249; 217 and 249; 218 and 249; 219 and 249; 220 and 249; 221 and 249; 222 and 249; 223 and 249; 224 and 249; 225 and 249; 226 and 249; 227 and 249; 228 and 249; 229 and 249; 230 and 249; 231 and 249.

In some aspects, the V_{H}-V_{L} pairs are selected from SEQ ID NOs: 206 and 250; 207 and 250; 208 and 250; 209 and 210; 211 and 250; 212 and 250; 213 and 250; 214 and 250; 215 and 250; 216 and 250; 217 and 250; 218 and 250; 219 and 250; 220 and 250; 221 and 250; 222 and 250; 223 and 250; 224 and 250; 225 and 250; 226 and 250; 227 and 250; 228 and 250; 229 and 250; 230 and 250; 231 and 250.

In some aspects, the V_{H}-V_{L} pairs are selected from SEQ ID NOs: 206 and 251; 207 and 251; 208 and 251; 209 and 210; 211 and 251; 212 and 251; 213 and 251; 214 and 251; 215 and 251; 216 and 251; 217 and 251; 218 and 251; 219 and 251; 220 and 251; 221 and 251; 222 and 251; 223 and 251; 224 and 251; 225 and 251; 226 and 251; 227 and 251; 228 and 251; 229 and 251; 230 and 251; 231 and 251.

In some aspects, the V_{H}-V_{L} pairs are selected from SEQ ID NOs: 206 and 252; 207 and 252; 208 and 252; 209 and 210; 211 and 252; 212 and 252; 213 and 252; 214 and 252; 215 and 252; 216 and 252; 217 and 252; 218 and 252; 219 and 252; 220 and 252; 221 and 252; 222 and 252; 223 and 252; 224 and 252; 225 and 252; 226 and 252; 227 and 252; 228 and 252; 229 and 252; 230 and 252; 231 and 252.

In some aspects, the V_{H}-V_{L} pairs are selected from SEQ ID NOs: 206 and 253; 207 and 253; 208 and 253; 209 and 210; 211 and 253; 212 and 253; 213 and 253; 214 and 253; 215 and 253; 216 and 253; 217 and 253; 218 and 253; 219 and 253; 220 and 253; 221 and 253; 222 and 253; 223 and 253; 224 and 253; 225 and 253; 226 and 253; 227 and 253; 228 and 253; 229 and 253; 230 and 253; 231 and 253.

In some aspects, the V_{H}-V_{L} pairs are selected from SEQ ID NOs: 206 and 254; 207 and 254; 208 and 254; 209 and 210; 211 and 254; 212 and 254; 213 and 254; 214 and 254; 215 and 254; 216 and 254; 217 and 254; 218 and 254; 219 and 254; 220 and 254; 221 and 254; 222 and 254; 223 and 254; 224 and 254; 225 and 254; 226 and 254; 227 and 254; 228 and 254; 229 and 254; 230 and 254; 231 and 254.

In some aspects, the V_{H}-V_{L} pairs are selected from SEQ ID NOs: 206 and 255; 207 and 255; 208 and 255; 209 and 210; 211 and 255; 212 and 255; 213 and 255; 214 and 255; 215 and 255; 216 and 255; 217 and 255; 218 and 255; 219 and 255; 220 and 255; 221 and 255; 222 and 255; 223 and 255; 224 and 255; 225 and 255; 226 and 255; 227 and 255; 228 and 255; 229 and 255; 230 and 255; 231 and 255.

2.7.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some embodiments, the antibody comprises a V_{H}-V_{L} pair, or a variant thereof, selected from the group consisting of SEQ ID NOS: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 2.7.5. Heavy Chain-Light Chain Pairs

In some embodiments, the antibody comprises a heavy chain sequence of an antibody disclosed herein and a light chain sequence of a suitable antibody. In some embodiments, the antibody comprises a heavy chain sequence of an antibody disclosed herein and a light chain sequence of an antibody disclosed herein.

In some embodiments, the heavy chain comprises a V_{H} sequence comprising, consisting of, or consisting essentially of any one of SEQ ID NOs: 207-231, and the light chain comprises a light chain sequence of any suitable antibody. Techniques for determining whether a particular light chain will pair with a heavy chain as described herein are well known to those of skill in the art. For example, a cell-free protein synthesis reaction comprising a nucleic acid encoding the heavy chain of interest and a nucleic acid encoding the light chain to be assessed may be performed as described, for example, in Example 1.

In some embodiments, the heavy chain comprises a V_{H} sequence comprising, consisting of, or consisting essentially of any one of SEQ ID NOs: 207-231, and the light chain comprises a V_{L} sequence comprising, consisting of, or consisting essentially of any one of SEQ ID NOs: 233-255.

In some embodiments, the heavy chain comprises a V_{H} sequence comprising, consisting of, or consisting essentially of any one of SEQ ID NOs: 207-231, and the light chain comprises a V_{L} sequence comprising, consisting of, or consisting essentially of any one of SEQ ID NOs: 233-255.

In some embodiments, the heavy chain comprises a V_{H} sequence comprising, consisting of, or consisting essentially of any one of SEQ ID NOs: 207-231, and the light chain comprises a V_{L} sequence comprising, consisting of, or consisting essentially of SEQ ID NO: 233-255.

### 2.8. Antibodies Comprising All Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some embodiments, the antibody comprises a CDR-H3, a CDR-H2, CDR-H1, a CDR-L3, a CDR-L2, and a CDR-L1 of a V_{H}-V_{L} pair selected from the group consisting of 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some embodiments, the antibody comprises a CDR-H3, a CDR-H2, CDR-H1, a CDR-L3, a CDR-L2, a CDR-L1, and framework regions of a V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some aspects, the CDR-H1 sequence is a Chothia CDR-H1 sequence comprising, consisting of, or consisting essentially of SEQ ID NOs: 5-29; the CDR-H2 sequence is a Chothia CDR-H2 sequence comprising, consisting of, or consisting essentially of SEQ ID NOs: 31-55; the CDR-H3 sequence is a CDR-H3 sequence comprising, consisting of, or consisting essentially of SEQ ID NOs: 109-133; the CDR-L1 sequence is a CDR-L1 sequence comprising, consisting of, or consisting essentially of SEQ ID NOs: 135-157; the CDR-L2 sequence is a CDR-L2 sequence comprising, consisting of, or consisting essentially of SEQ ID NOs: 159-181; and the CDR-L3 sequence is a CDR-L3 sequence comprising, consisting of, or consisting essentially of SEQ ID NOs: 183-205.

In some aspects, the CDR-H1 sequence is a Kabat CDR-H1 sequence comprising, consisting of, or consisting essentially of SEQ ID NOs: 31-55; the CDR-H2 sequence is a Kabat CDR-H2 sequence comprising, consisting of, or consisting essentially of SEQ ID NOs: 83-107; the CDR-H3 sequence is a CDR-H3 sequence comprising, consisting of, or consisting essentially of SEQ ID NOs: 109-133; the CDR-L1 sequence is a CDR-L1 sequence comprising, consisting of, or consisting essentially of SEQ ID NOs: 135-157; the CDR-L2 sequence is a CDR-L2 sequence comprising, consisting of, or consisting essentially of SEQ ID NOs: 159-181; and the CDR-L3 sequence is a CDR-L3 sequence comprising, consisting of, or consisting essentially of SEQ ID NOs: 183-205.

In some embodiments, the antibody comprises six CDRs selected from the group consisting of SEQ ID NOs: 9/61/113/134/163/187; 9/61/113/138/162/186; 9/61/113/137/161/185; 9/61/113/136/160/184; 9/61/113/135/159/183; 8/60/112/134/163/187; 8/60/112/138/162/186; 8/60/112/137/161/185; 8/60/112/136/160/184; 8/60/112/135/159/183; 7/59/111/134/163/187; 7/59/111/138/162/186; 7/59/111/137/161/185; 7/59/111/136/160/184; 7/59/111/135/159/183; 6/58/110/134/163/187; 6/58/110/138/162/186; 6/58/110/137/161/185; 6/58/110/136/160/184; 6/58/110/135/159/183; 5/57/109/134/163/187; 5/57/109/138/162/186; 5/57/109/137/161/185; 5/57/109/136/160/184; 5/57/109/135/159/183; 11/63/115/142/166/190; 11/63/115/141/165/189; 11/63/115/140/164/188; 10/62/114/142/166/190; 12/64/116/142/166/190; 13/65/117/142/166/190; 14/66/118/142/166/190; 15/67/119/142/166/190; 16/68/120/142/166/190; 18/70/122/142/166/190; 19/71/123/142/166/190; 20/72/124/142/166/190; 21/73/125/142/166/190; 22/74/126/142/166/190; 23/75/127/142/166/190; 24/76/128/142/166/190; 25/77/129/142/166/190; 26/78/130/142/166/190; 27/79/131/142/166/190; 28/80/132/142/166/190; 29/81/133/142/166/190; 10/62/114/143/166/191; 10/62/114/144/167/192; 10/62/114/145/168/193; 10/62/114/146/169/194; 10/62/114/147/170/195; 6/58/110/157/181/205; 6/58/110/156/180/204; 6/58/110/155/179/203; 6/58/110/154/178/202; 6/58/110/153/177/201; 6/58/110/152/176/200; 6/58/110/151/175/199; 6/58/110/150/174/198; 6/58/110/149/173/197; 6/58/110/148/172/196; 17/69/121/143/166/191; 14/66/118/143/166/191; 12/64/116/143/166/191; 9/61/113/143/166/191; 24/76/128/143/166/191; 17/69/121/143/166/191; 14/66/118/143/166/191; 12/64/116/143/166/191; 9/61/113/143/166/191; 24/76/128/143/166/191; 17/69/121/153/177/201; 14/66/118/153/177/201; 12/64/116/153/177/201; 9/61/113/153/177/201; 14/66/118/153/177/201; 18/70/122/152/176/200; 14/66/118/152/176/200; 12/64/116/152/176/200; 9/61/113/152/176/200; and 24/76/128/152/176/200.

In some embodiments, the antibody comprises six CDRs selected from the group consisting of SEQ ID NOs: 35/87/113/134/163/187; 35/87/113/138/162/186; 35/87/113/137/161/185; 35/87/113/136/160/184; 35/87/113/135/159/183; 34/86/112/134/163/187; 34/86/112/138/162/186; 34/86/112/137/161/185; 34/86/112/136/160/184; 34/86/112/135/159/183; 33/85/111/134/163/187; 33/85/111/138/162/186; 33/85/111/137/161/185; 33/85/111/136/160/184; 33/85/111/135/159/183; 32/84/110/134/163/187; 32/84/110/138/162/186; 32/84/110/137/161/185; 32/84/110/136/160/184; 32/84/110/135/159/183; 31/83/109/134/163/187; 31/83/109/138/162/186; 31/83/109/137/161/185; 31/83/109/136/160/184; 31/83/109/135/159/183; 37/89/115/142/166/190; 37/89/115/141/165/189; 37/89/115/140/164/188; 36/88/114/142/166/190; 38/64/90/142/166/190; 39/91/117/142/166/190; 40/92/118/142/166/190; 41/93/119/142/166/190; 42/94/120/142/166/190; 44/96/122/142/166/190; 45/97/123/142/166/190; 46/98/124/142/166/190; 47/99/125/142/166/190; 48/100/126/142/166/190; 49/101/127/142/166/190; 50/102/128/142/166/190; 51/103/129/142/166/190; 52/104/130/142/166/190; 53/105/131/142/166/190; 54/106/132/142/166/190; 55/107/133/142/166/190; 36/88/114/143/166/191; 36/88/114/144/167/192; 36/88/114/145/168/193; 36/88/114/146/169/194; 36/88/114/147/170/195; 32/84/110/157/181/205; 32/84/110/156/180/204; 32/84/110/155/179/203; 32/84/110/154/178/202; 32/84/110/153/177/201; 32/84/110/152/176/200; 32/84/110/151/175/199; 32/84/110/150/174/198; 32/84/110/149/173/197; 32/84/110/148/172/196; 43/95/121/143/166/191; 40/92/118/143/166/191; 38/64/90/143/166/191; 35/87/113/143/166/191; 50/102/128/143/166/191; 43/95/121/143/166/191; 40/92/118/143/166/191; 38/64/90/143/166/191; 35/87/113/143/166/191; 50/102/128/143/166/191; 43/95/121/153/177/201; 40/92/118/153/177/201; 38/64/90/153/177/201; 35/87/113/153/177/201; 50/102/128/153/177/201; 44/96/122/152/176/200; 40/92/118/152/176/200; 38/64/90/152/176/200; 35/87/113/152/176/200; and 50/102/128/152/176/200.

### 2.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, 95%, 96%, 97%, 98%, or 99% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, 95%, 96%, 97%, 98%, or 99% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, 95%, 96%, 97%, 98%, or 99% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 2.9. Consensus Sequences

In some embodiments, provided herein are anti-CD3 antibodies comprising one or more sequences defined by consensus sequences. Each consensus sequence is based, at least in part, on one or more alignments of two or more useful anti-CD3 CDR sequences provided in this disclosure. Based on such alignments, a person of skill in the art would recognize that different amino acid residues may useful in certain positions of the CDRs. Accordingly, each consensus sequence encompasses two or more useful anti-CD3 CDR sequences.

In some embodiments, the antibodies comprise one to six of the consensus CDR sequences provided herein. In some embodiments, the antibodies comprise two to six of the consensus CDR sequences provided herein. In some embodiments, the antibodies comprise three to six of the consensus CDR sequences provided herein. In some embodiments, the antibodies comprise four to six of the consensus CDR sequences provided herein. In some embodiments, the antibodies comprise five to six of the consensus CDR sequences provided herein. In some embodiments, the antibodies comprise six of the consensus CDR sequences provided herein. In some embodiments, the antibodies comprise a V_{L} comprising the CDR-L consensus sequence(s). In some embodiments, the antibodies comprise a V_{H} comprising the CDR-H consensus sequence(s). In some embodiments, the antibodies comprise a V_{H} comprising the CDR-H consensus sequence(s) and a V_{L} comprising the CDR-L consensus sequence(s).

### 2.9.1. CDR-H3 Consensus Sequences

In some embodiments, the antibody comprises a CDR-H3 sequence defined by the consensus sequence H-G-α₁-α₂-α₃-α₄-α₅-α₆-I-S-Y-W-A-Y, where α₁ is N or D; α₂ is F or D; α₃ is G or D; α₄ is N or D; α₅ is S or D; α₆ is Y or D (SEQ ID NO: 294).

In some embodiments, the sequence is selected from SEQ ID NOs: 109-133, or a variant thereof having three, two, or one amino acid substitution(s).

### 2.9.2. Chothia CDR-H1 Consensus Sequences

In some embodiments, the antibody comprises a Chothia CDR-H1 sequence defined by the consensus sequence G-ε₁-T-ε₂-ε₃-ε₄-Y, wherein ε₁ is F or D; ε₂ is F or D; ε₃ is N or D; and ε₆ is T or K.

In some embodiments, the sequence is selected from SEQ ID NOs:5-29, or a variant thereof having two or one amino acid substitutions(s).

### 2.9.3. Chothia CDR-H2 Consensus Sequences

In some embodiments, the antibody comprises a Chothia CDR-H2 sequence defined by the consensus sequence ζ1-ζ₂-ζ₃-ζ₄-ζ₅-N-Y-A, wherein ζ₁ is R or D; ζ₂ is S or T; ζ₃ is K or D; ζ₄ is Y or D; and ζ₅ is N or D.

In some embodiments, the sequence is selected from SEQ ID NOs: 57-81, or a variant thereof having two or one amino acid substitutions.

In some embodiments, ζ₂ is T.

### 2.9.4. Kabat CDR-H1 Consensus Sequences

In some embodiments, the antibody comprises a Kabat CDR-H1 sequence defined by the consensus sequence μ₁-Y-μ₂-M-N, where μ₁ is K or D; μ₂ is A or D.

In some embodiments, the sequence is selected from SEQ ID NOs: 31-55, or a variant thereof having two or one amino acid substitutions(s).

### 2.9.5. Kabat CDR-H2 Consensus Sequences

In some embodiments, the antibody comprises a Kabat CDR-H2 sequence defined by the consensus sequence θ₁-I-θ₃-θ₄-θ₅-θ₆-θ₇-N-Y-θ₁₀-T-θ₁₁-Y-A-D-S-V-K-D; wherein θ₁ is R or D; θ₃ is R or D; θ₄ is S or T; θ₅ is K or D; θ₆ is Y or D; θ₇ is N or D; θ₁₀ is A or D; and θ₁₁ is Y or D (SEQ ID NO: 295).

In some embodiments, a sequence is selected from SEQ ID NOs: 83-107, or a variant thereof having three, two, or one amino acid substitutions(s).

In some embodiments, θ₄ is T.

### 2.9.6. Kabat CDR-L3 Consensus Sequences

In some embodiments, the antibody comprises a Kabat CDR-H2 sequence defined by the sequence the consensus sequence A-L-β₃-β₄-β₅-β₆-β₇-W-V, where β₃ is W or D; β₄ is Y, F, or D, β₅ is S or D, β₆ is N or D, and β₇ is R or D (SEQ ID NO: 296).

In some embodiments, the sequence is selected from SEQ ID NOs: 183-205, or a variant thereof having three, two, or one amino acid substitution(s).

In some embodiments, β7 is R.

### 2.9.7. Kabat CDR-L2 Consensus Sequences

In some embodiments, the antibody comprises a Kabat CDR-H2 sequence defined by the sequence defined by the consensus sequence G-T-K-F-L-A-P (SEQ ID NO: 299).

In some embodiments, the sequence is selected from SEQ ID NOs: 159-181.

### 2.9.8. Kabat CDR-L1 Consensus Sequences

In some embodiments, the antibody comprises a Kabat CDR-H2 sequence defined by the sequence defined by the consensus sequence G-S-S-T-G-A-V-T-S-G-Y-Y-P-N (SEQ ID NO: 300).

In some embodiments, the sequence is selected from SEQ ID NOs: 135-157.

### 2.9.8.1 Constant Region Sequences

In some embodiments, the antibody of any one of the preceding embodiments comprises at least one constant region domain.

In some embodiments, the antibody, of any one of the preceding embodiments, comprises a constant region comprises a sequence selected from SEQ ID Nos: 257-259.

### 2.9.9. Framework Regions

In some embodiments, the antibody of any of the previous embodiments further comprises human framework regions.

In some embodiments, the antibody of any of the previous embodiments, further comprises human kappa framework regions.

### 3. scFv

In some embodiments, the antibody comprises an scFv sequence comprising, consisting of, or consisting essentially of a scFv sequence provided herein. In some aspects, the scFv sequence is an scFv sequence provided in SEQ ID NOs: 264-290.

In some embodiments, the antibody comprises an scFv sequence comprising, consisting of, or consisting essentially of a sequence selected from SEQ ID NOs: 264-290. In some aspects, the antibody comprises an scFv sequence comprising, consisting of, or consisting essentially of SEQ ID NO: 264. In some aspects, the antibody comprises an scFv sequence comprising, consisting of, or consisting essentially of SEQ ID NO: 265. In some aspects, the antibody comprises an scFv sequence comprising, consisting of, or consisting essentially of SEQ ID NO: 266. In some aspects, the antibody comprises an scFv sequence comprising, consisting of, or consisting essentially of SEQ ID NO: 267. In some aspects, the antibody comprises an scFv sequence comprising, consisting of, or consisting essentially of SEQ ID NO: 268. In some aspects, the antibody comprises an scFv sequence comprising, consisting of, or consisting essentially of SEQ ID NO: 269. In some aspects, the antibody comprises an scFv sequence comprising, consisting of, or consisting essentially of SEQ ID NO: 270. In some aspects, the antibody comprises an scFv sequence comprising, consisting of, or consisting essentially of SEQ ID NO: 271. In some aspects, the antibody comprises an scFv sequence comprising, consisting of, or consisting essentially of SEQ ID NO: 272. In some aspects, the antibody comprises an scFv sequence comprising, consisting of, or consisting essentially of SEQ ID NO: 273. In some aspects, the antibody comprises an scFv sequence comprising, consisting of, or consisting essentially of SEQ ID NO: 274. In some aspects, the antibody comprises an scFv sequence comprising, consisting of, or consisting essentially of SEQ ID NO: 275. In some aspects, the antibody comprises an scFv sequence comprising, consisting of, or consisting essentially of SEQ ID NO: 276. In some aspects, the antibody comprises an scFv sequence comprising, consisting of, or consisting essentially of SEQ ID NO: 277. In some aspects, the antibody comprises an scFv sequence comprising, consisting of, or consisting essentially of SEQ ID NO: 278. In some aspects, the antibody comprises an scFv sequence comprising, consisting of, or consisting essentially of SEQ ID NO: 279. In some aspects, the antibody comprises an scFv sequence comprising, consisting of, or consisting essentially of SEQ ID NO: 280. In some aspects, the antibody comprises an scFv sequence comprising, consisting of, or consisting essentially of SEQ ID NO: 281. In some aspects, the antibody comprises an scFv sequence comprising, consisting of, or consisting essentially of SEQ ID NO: 282. In some aspects, the antibody comprises an scFv sequence comprising, consisting of, or consisting essentially of SEQ ID NO: 283. In some aspects, the antibody comprises an scFv sequence comprising, consisting of, or consisting essentially of SEQ ID NO: 284. In some aspects, the antibody comprises an scFv sequence comprising, consisting of, or consisting essentially of SEQ ID NO: 285. In some aspects, the antibody comprises an scFv sequence comprising, consisting of, or consisting essentially of SEQ ID NO: 286. In some aspects, the antibody comprises an scFv sequence comprising, consisting of, or consisting essentially of SEQ ID NO: 287. In some aspects, the antibody comprises an scFv sequence comprising, consisting of, or consisting essentially of SEQ ID NO: 288. In some aspects, the antibody comprises an scFv sequence comprising, consisting of, or consisting essentially of SEQ ID NO: 289. In some aspects, the antibody comprises an scFv sequence comprising, consisting of, or consisting essentially of SEQ ID NO: 290.

In some aspects, the scFv sequence comprises, consists of, or consists essentially of a variant of an illustrative scFv sequence provided in this disclosure. In some aspects, the scFv sequence comprises, consists of, or consists essentially of a sequence having at least 70%, 75%, 80%, 85%, 90%, 95%, 96%, 97%, 98%, or 99% identity with any of the illustrative scFv sequences provided in this disclosure. In some aspects, the scFv sequence comprises, consists of, or consists essentially of any of the illustrative scFv sequences provided in this disclosure, with 1, 2, or 3 amino acid substitutions. In some aspects, the amino acid substitutions are conservative amino acid substitutions.

### 4. Germline

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 1, V_{H} 2, V_{H} 3, or V_{H} 4 families, or a variant thereof. In some embodiments, a CDR-L sequence provided herein may be combined with a variable region encoded by a variable region germline gene selected from the Vκ1, Vκ2, or Vκ3, or a variant thereof. 5. Affinity

In some embodiments, the affinity of the antibody for CD3 as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, less than about 10⁻¹² M. In some embodiments, the affinity of the antibody is between about 10⁻⁷ M and 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹² M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁸ M and 10⁻⁹ M. In some embodiments, the affinity of the antibody is between about 10⁻⁹ M and 10⁻¹² M. In some embodiments, the affinity of the antibody is between about 10⁻⁹ M and 10⁻¹¹ M. In some embodiments, the affinity of the antibody is between about 10⁻⁹ M and 10⁻¹⁰ M. In some embodiments, the affinity of the antibody is between about 10⁻¹⁰ M and 10⁻¹² M. In some embodiments, the affinity of the antibody is between about 10⁻¹¹ M and 10⁻¹² M.

In some embodiments the antibody has a kₐ of at least about 10³ M⁻¹×sec⁻¹. In some embodiments the antibody has a kₐ of at least about 10⁴ M⁻¹×sec⁻¹. In some embodiments the antibody has a kₐ of at least about 10⁵ M⁻¹×sec⁻¹. In some embodiments the antibody has a kₐ of at least about 10⁶ M⁻¹×sec⁻¹. In some embodiments the antibody has a kₐ of between about 10³ M⁻¹×sec⁻¹ and about 10⁷ M⁻¹×sec⁻¹. In some embodiments the antibody has a kₐ of between about 10³ M⁻¹×sec⁻¹ and about 10⁴ M⁻¹×sec⁻¹. In some embodiments the antibody has a kₐ of between about 10³ M⁻¹×sec⁻¹ and about 10⁵ M⁻¹×sec⁻¹. In some embodiments the antibody has a kₐ of between about 10³ M⁻¹×sec⁻¹ and about 10⁶ M⁻¹×sec⁻¹. In some embodiments the antibody has a kₐ of between about 10⁴ M⁻¹×sec⁻¹ and about 10⁵ M⁻¹×sec⁻¹. In some embodiments the antibody has a kₐ of between about 10⁴ M⁻¹×sec⁻¹ and about 10⁶ M⁻¹×sec⁻¹. In some embodiments the antibody has a kₐ of between about 10⁴ M⁻¹×sec⁻¹ and about 10⁷ M⁻¹×sec⁻¹. In some embodiments the antibody has a kₐ of between about 10⁵ M⁻¹×sec⁻¹ and about 10⁶ M⁻¹×sec⁻¹. In some embodiments the antibody has a kₐ of between about 10⁵ M⁻¹×sec⁻¹ and about 10⁷ M⁻¹×sec⁻¹. In some embodiments the antibody has a kₐ of between about 10⁶ M⁻¹×sec⁻¹ and about 10⁷ M⁻¹×sec¹.

In some embodiments the antibody has a k_{d} of about 10⁻⁸ sec⁻¹ or less. In some embodiments the antibody has a k_{d} of about 10⁻⁷ sec⁻¹ or less. In some embodiments the antibody has a k_{d} of about 10⁻⁶ sec⁻¹ or less. In some embodiments the antibody has a k_{d} of about 10⁻⁵ sec¹ or less. In some embodiments the antibody has a k_{d} of about 10˜⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁸ sec⁻¹. In some embodiments the antibody has a k_{d} of between about 10⁻² sec⁻¹ and about 10⁻⁷ sec⁻¹. In some embodiments the antibody has a k_{d} of between about 10⁻² sec⁻¹ and about 10⁻⁶ sec⁻¹. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 10⁻⁴ sec⁻¹. In some embodiments the antibody has a k_{d} of between about 10⁻² sec⁻¹ and about 10⁻³ sec⁻¹. In some embodiments the antibody has a k_{d} of between about 10⁻³ sec⁻¹ and about 10⁻⁸ sec⁻¹. In some embodiments the antibody has a k_{d} of between about 10⁻³ sec⁻¹ and about 10⁻⁷ sec⁻¹. In some embodiments the antibody has a k_{d} of between about 10⁻³ sec⁻¹ and about 10⁻⁶ sec⁻¹. In some embodiments the antibody has a k_{d} of between about 10⁻³ sec⁻¹ and about 10⁻⁵ sec⁻¹. In some embodiments the antibody has a k_{d} of between about 10⁻³ sec⁻¹ and about 10⁻⁴ sec⁻¹. In some embodiments the antibody has a k_{d} of between about 10⁻⁴ sec⁻¹ and about 10⁻⁸ sec⁻¹. In some embodiments the antibody has a k_{d} of between about 10⁻⁴ sec⁻¹ and about 10⁻⁷ sec⁻¹. In some embodiments the antibody has a k_{d} of between about 10⁻⁴ sec⁻¹ and about 10⁻⁶ sec⁻¹. In some embodiments the antibody has a k_{d} of between about 10⁻⁴ sec⁻¹ and about 10⁻⁵ sec⁻¹. In some embodiments the antibody has a k_{d} of between about 10⁻⁵ sec⁻¹ and about 10⁻⁸ sec⁻¹. In some embodiments the antibody has a k_{d} of between about 10⁻⁵ sec⁻¹ and about 10⁻⁷ sec⁻¹. In some embodiments the antibody has a k_{d} of between about 10⁻⁵ sec⁻¹ and about 10⁻⁶ sec⁻¹. In some embodiments the antibody has a k_{d} of between about 10⁻⁶ sec⁻¹ and about 10⁻⁸ sec⁻¹. In some embodiments the antibody has a k_{d} of between about 10⁻⁶ sec⁻¹ and about 10⁻⁷ sec⁻¹. In some embodiments the antibody has a k_{d} of between about 10⁻⁷ sec⁻¹ and about 10⁻⁸ sec⁻¹.

In some embodiments, the affinity of the antibody for human CD3, human CD3 extracellular domain, or for individual domains within human CD3, as determined by surface plasmon resonance at 25° C., and as indicated by K_{D}, is from about 1.49×10⁻⁹ M to about 1.85×10⁻⁷ M. In some embodiments, the affinity of the antibody for human CD3, as determined by surface plasmon resonance at 25° C., and as indicated by K_{D}, is from about 7.15×10⁻⁹ M to about 1.22×10⁻⁸ M. In some embodiments, the affinity of the antibody for human CD3, as determined by surface plasmon resonance at 25° C., and as indicated by K_{D}, is from about 8.50×10⁻⁸ M to about 9.23×10⁻¹² M. In some embodiments, the affinity of the antibody for human CD3 is about any of the K_{D} values reported for human CD3 in the examples below.

In some embodiments the antibody has a kₐ when associating with human CD3, human CD3 extracellular domain, or for individual domains within human CD3, as determined by surface plasmon resonance at 25° C., of from about 4.11×10³ M⁻¹×sec⁻¹ to about 2.44×10⁵ M⁻¹×sec⁻¹. In some embodiments the antibody has a kₐ when associating with human CD3, as determined by surface plasmon resonance at 25° C., of from about 3.82×10⁴ M⁻¹×sec⁻¹ to about 6.77×10⁵ M⁻¹×sec⁻¹. In some embodiments the antibody has a kₐ when associating with human CD3, as determined by surface plasmon resonance at 25° C., of from about 7.72×10³ M⁻¹×sec⁻¹ to about 7.96×10⁵ M⁻¹×sec⁻¹. In some embodiments the antibody has a kₐ when associating with human CD3 of about any of the kₐ values reported for human CD3 in the examples below.

In some embodiments the antibody has a k_{d} when dissociating from human CD3, human CD3 extracellular domain, or for individual domains within human CD3, as determined by surface plasmon resonance at 25° C., of from about 1.15×10⁻⁴ sec⁻¹ to about 7.61×10⁻⁴ sec⁻¹. In some embodiments the antibody has a k_{d} when dissociating from human CD3, as determined by surface plasmon resonance at 25° C., of from about 4.64×10−4 sec⁻¹ to about 2.78×10−4 sec⁻¹. In some embodiments the antibody has a k_{d} when dissociating from human CD3, as determined by surface plasmon resonance at 25° C., of from about 1.90×10−4 sec⁻¹ to about 7.12×10⁻⁸ sec⁻¹. In some embodiments the antibody has a k_{d} when dissociating from human CD3 of about any of the k_{d} values reported for human CD3 in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein. 6. Epitope Bins

In some embodiments, the antibody binds the same epitope as an antibody encompassing any of SEQ ID NOs: 207-255. In some embodiments, the antibody binds the same epitope as an antibody comprising any of the V_{H}-V_{L} pairs, above. In some embodiments, the antibody competes for epitope binding with an antibody encompassing any of SEQ ID NOs: 207-255. In some embodiments, the antibody competes for epitope binding with an antibody comprising any of the V_{H}-V_{L} pairs, above.

In some embodiments, the antibody specifically binds to CD3, wherein the antibody binds to the same epitope as a second antibody with a V_{H} region selected from SEQ ID NOs: 207-231 and a V_{L} region selected from SEQ ID NOs: 233-255.

### 7. Glycosylation Variants

In certain embodiments, an antibody may be altered to increase, decrease or eliminate the extent to which it is glycosylated. In certain embodiments, an antibody may be altered to be aglycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 8. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

An alteration in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 9. Preparation of Antibodies

### 9.1. Antigen Preparation

The CD3 protein to be used for isolation of the antibodies may be intact CD3 or a fragment of CD3. The intact CD3 protein, or fragment of CD3, may be in the form of an isolated protein or protein expressed by a cell. Other forms of CD3 useful for generating antibodies will be apparent to those skilled in the art.

### 9.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies. 9.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

In some embodiments, an isolated antibody that binds to CD3 has been humanized.

In some embodiments, an isolated humanized antibody that specifically binds to CD3, comprises a CDR-H3 sequence defined by the consensus sequence defined by the consensus sequence H-G-α₁-α₂-α₃-α₄-α₅-α₆-I-S-Y-W-A-Y, where α₁ is N or D; α₂ is F or D; α₃ is G or D; α₄ is N or D; α₅ is S or D; α₆ is Y or D (SEQ ID NO: 294), and selected from SEQ ID NOs: 109-133. In some embodiments, the isolated humanized antibody comprises a CDR-H1 sequence defined by the consensus sequence μ₁-Y-μ₂-M-N, where μ₁ is K or D; μ₂ is A or D, and selected from SEQ ID NOs: 31-55 or defined by the consensus sequence a sequence G-ε₁-T-ε₂-ε₃-ε₄-Y, wherein ε₁ is F or D; ε₂ is F or D; ε₃ is N or D; and ε₄ is K or D, and selected from SEQ ID NOs: 5-29. In some embodiments, the isolated humanized antibody comprises a CDR-H2 sequence defined by a sequence defined by the consensus sequence θ₁-I-θ₃-θ₄-θ₅-θ₆-θ₇-N-Y-θ₁₀-T-θ₁₁-Y-A-D-S-V-K-D; wherein θ₁ is R or D; θ₃ is R or D; θ₄ is S or T; θ₅ is K or D; θ₆ is Y or D; θ₇ is N or D; θ₁₀ is A or D; and θ₁₁ is Y or D; and θ₁₁ is Y or D (SEQ ID NO: 301), and sequence selected from SEQ ID NOs: 83-107, or a sequence defined by the consensus sequence ζ₁-ζ₂-ζ₃-ζ₄-ζ₅-N-Y-A, wherein ζ₁ is R or D; ζ₂ is S or T; ζ₃ is K or D; ζ₄ is Y or D; and ζ₅ is N or D and selected from SEQ ID NOs: 57-81. In certain embodiments, ζ₂ is T. In certain embodiments, θ₄ is T. In some embodiments, the isolated humanized antibody comprises a CDR-L1 sequence defined by the consensus sequence G-S-S-T-G-A-V-T-S-G-Y-Y-P-N (SEQ ID NO: 300) and selected from SEQ ID NOs: 135-157. In some embodiments, the isolated humanized antibody comprises a CDR-L2 sequence defined by defined by the consensus sequence G-T-K-F-L-A-P (SEQ ID NO: 299) and selected from SEQ ID NOs: 159-181. In some embodiments, the isolated humanized antibody comprises a CDR-L3 sequence a sequence defined by the consensus sequence A-L-β₃-β₄-β₅-β₆-β₇-W-V, where β₃ is W or D; β₄ is Y, F, or D, β₅ is S or D, β₆ is N or D, and β₇ is R or D (SEQ ID NO: 296) and selected from SEQ ID NOs: 183-205. In some embodiments, the isolated humanized antibody comprises a V_{H} having the sequence set forth in one of SEQ ID NOs: 207-231. In certain embodiments, the isolated humanized antibody comprises a V_{L} having the sequences set forth in one of SEQ ID NOs: 232-255. In certain embodiments, β₇ is R.

In some embodiments, the humanized antibody comprises a V_{H} comprises four framework regions. In some embodiments, a first framework region is defined by E-V-Q-L-γ₅-E-S-G-G-G-L-V-γ₁₃-P-G-T₁₆-S-L-γ₁₉-L-S-C-γ₂₃-A-S, wherein γ₅ is L or V, γ₁₃ is K or Q, γ₁₆ is G or R, γ₁₉ is K or R; γ₂₃ is T or A (SEQ ID NO: 302). In some embodiments, a second framework region defined by the sequence W-γ₃₇-R-Q-A-γ₄₁-G-K-G-L-E-W-V-A; γ₃₇ is V or F, γ₄₁ is S or P (SEQ ID NO: 303). In some embodiments, a third frame work region is defined by the sequence R-F-T-I-S-R-D-D-S-K-γ₇₉-γ₈₀-A-Y-L-Q-M-N-γ₈₇-L-γ₈₉-γ₉₀-E-D-T-A-V-Y-Y-C-V-R (SEQ ID NO: 304); wherein γ₇₉ is S or N, γ₈₀ is I or T, γ₈₇ is S or N, γ₈₉ is R or K, and γ₉₀ A or T. In some embodiments, a fourth framework region is defined by the sequence W-G-Q-G-T-γ₁₁₈-V-T-V-S-S; wherein γ₁₁₈ is P or L (SEQ ID NO: 305).

In some embodiments, the V_{L} comprises four framework regions. In some embodiments, a first framework region is defined by the sequence £₁-£₂-£₃-£₄-T-Q-£₇-£₈-£₉-£₁₀-£₁₁-£₁₂-£₁₃-£₁₄-£₁₅-£₁₆-£₁₇-£₁₈-£₁₉-£₂₀-£₂₁-£₂₂; wherein £₁ is Q, E, or D; £₂ is T, I, or S; £₃ is Q, V, and A; £₄ is M or V; £₇ is S, E, and P; £₈ is P or R; £₉ is S or G; £₁₀ is F, L, V, or T; i is S, T, or L; £₁₂ is V, G, S, or A; £₁₃ is S or L; £₁₄ is P, S, or V; £₁₅ is G or P; £₁₆ is G, Q, or D; £₁₇ is T, S, E, or R; £₁₈ is V or R; £₁₉ is T or A; £₂₀ is L, I, or T; £₂₁ is T, S, or L; and £₂₂ is C or S. In some embodiments, a second framework region defined by the sequence W-£₃₈-Q-Q-£₄₁-P-G-£₄₄-A-P-£₄₇-£₄₈-£₄₉-I-G (SEQ ID NO: 306); wherein £₃₈ is Y, F, V, or L; £₄₁ is T, K, or H; £₄₄ is Q or K; £₄₇ is R or K; £₄₈ is G or L; £₄₉ is L or M. In some embodiments, a third framework region defined by the sequence G-£₆₀-P-£₆₂-R-F-S-G-S-£₆₈-£₆₉-G-£₇₁-£₇₂-£₇₃-£₇₄-L-T-£₇₇-£₇₈-£₇₉-£₈₀-£₈₁-£₈₂-£₈₃-D-£₈₅-£₈₆-£₈₇-Y-Y-C (SEQ ID NO: 307); wherein £₆₀ is V, T, or I; £₆₂ is D, A, or S; £₆₈ is L or G; £₆₉ L or S; £₇₁ is T, G, or N; £₇₂ is K, T, or D; £₇₃ is A or F; £₇₄ is A, S, or T; £₇₇ is L or I; £₇₈ is T or S; £₇₉ is G, R, or S; £₈₀ is A, V, or L; £₈₁ is Q or E; £₈₂ is A or P; £₈₃ is D or E; £₈₅ is F or E; £₈₆ is S or A; £₈₇ is T, E, V, or D. In some embodiments, a fourth framework region defined by the sequence £₁₀₀-G-£₁₀₂-G-£₁₀₄-£₁₀₅-£₁₀₆-£₁₀₇-£₁₀₈-£₁₀₉; wherein £₁₀₀ is F or G; £₁₀₂ is G, S, or Q; £₁₀₄ is T or Q; £₁₀₅ is Q or K; £₁₀₆ is V or L; £₁₀₇ is T or E; £₁₀₈ is I or V; £₁₀₉ is T or K.

In some embodiments, the humanized antibody of the first framework region of the V_{L} has a serine insertion between £₉-£₁₀.

In some embodiments, the isolated humanized antibody that specifically binds to CD3, comprises an antibody V_{H} comprising one or more of the following residues: D27, D29, D30, D31, D33, D50, D54, D55, D56, D59, D61, D101, D102, D104, D105, and D106.

In some embodiments, the isolated humanized antibody that specifically binds to CD3, comprises an antibody V_{L} comprising one or more of the following residues: D93, D94, D95, D96, and D97.

### 9.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 10. Vectors, Host Cells, and Recombinant Methods

The invention also provides isolated nucleic acids encoding anti-CD3 antibodies, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-CD3 antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-CD3 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ⁴ heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt). 11. Pharmaceutical Compositions and Methods of Administration

Any of the antibodies provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

### 11.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 11.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months. 12. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving CD3. In some embodiments, the disease or condition is a disease or condition that can be diagnosed by overexpression of CD3. In some embodiments, the disease or condition is a disease or condition that can benefit from treatment with an anti-CD3 antibody. In some embodiments, the disease or condition is a cancer.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibodies provided herein is gastric cancer, colorectal cancer, renal cell carcinoma, cervical cancer, non-small cell lung carcinoma, ovarian cancer, uterine cancer, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is lung cancer. In some embodiments, the disease is head and neck cancer. In some embodiments, the disease is renal cell carcinoma. In some embodiments, the disease is brain carcinoma. In some embodiments, the disease is endometrial carcinoma.

### 13. Diagnostic Applications

In some embodiments, the antibodies provided herein are used in diagnostic applications. For example, an anti-CD3 antibody may be useful in assays for CD3 protein. In some aspects the antibody can be used to detect the expression of CD3 in various cells and tissues. These assays may be useful, for example, in making a diagnosis and/or prognosis for a disease, such as a cancer.

In some diagnostic and prognostic applications, the antibody may be labeled with a detectable moiety. Suitable detectable moieties include, but are not limited to radioisotopes, fluorescent labels, and enzyme-substrate labels. In another embodiment, the anti-CD3 antibody need not be labeled, and the presence of the antibody can be detected using a labeled antibody which specifically binds to the anti-CD3 antibody.

### 14. Affinity Purification Reagents

The antibodies of the invention may be used as affinity purification agents. In this process, the antibodies may be immobilized on a solid phase such a resin or filter paper, using methods well known in the art. The immobilized antibody is contacted with a sample containing the CD3 protein (or fragment thereof) to be purified, and thereafter the support is washed with a suitable solvent that will remove substantially all the material in the sample except the CD3 protein, which is bound to the immobilized antibody. Finally, the support is washed with another suitable solvent, such as glycine buffer, pH 5.0 that will release the CD3 protein from the antibody.

### 15. Kits

In some embodiments, an anti-CD3 antibody provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-CD3 antibody. In some embodiments, the anti-CD3 antibody is provided in the form of a pharmaceutical composition. In some embodiments, the anti-CD3 antibody is provided in a lyophilized form.

### 16. Bi-Specific Antibodies

In some embodiments, the isolated antibody that binds CD3 is provided as a single arm binder. For example, the anti-CD3 antibody is provided as part of a bi-specific antibody or bi-specific antibody construct as disclosed here.

### 16.1. Bi-Specific Antibodies and Antigen-Binding Constructs

Provided herein are bi-specific antigen-binding constructs, e.g., antibodies, that bind to CD3 and a different antigen. The bi-specific antigen-binding construct includes two antigen-binding polypeptide constructs, e.g., antigen binding domains, that specifically binding to CD3 and/or another antigen. In some embodiments, the antigen-binding construct is derived from known antibodies or antigen-binding constructs. In some embodiments, the antigen-binding polypeptide constructs comprise two antigen binding domains that comprise antibody fragments. The antibody fragments may be the same format or different formats from each other. For example, in some embodiments, the antigen-binding polypeptide constructs comprise a first antigen binding domain comprising an scFv and a second antigen binding domain comprising a Fab.

In some embodiments, the antigen-binding polypeptide constructs comprise a first antigen binding domain and a second antigen binding domain, wherein both antigen binding domains comprise an scFv. In other embodiments, the first and second antigen binding domains each comprise a Fab. Any combination of antibody formats is suitable for the bi-specific antibody constructs disclosed herein.

In some embodiments, a bi-specific antibody or antigen-binding construct comprises a first binding domain that specifically binds to CD3, and a second binding domain that specifically binds to a different antigen. In some embodiments, the first binding domain comprises a CDR-H3 sequence defined by the consensus sequence H-G-α₁-α₂-α₃-α₄-α₅-α₆-I-S-Y-W-A-Y (SEQ ID NO: 294), where α₁ is N or D; α₂ is F or D; α₃ is G or D; α₄ is N or D; α₅ is S or D; α₆ is Y or D. In some embodiments, the bi-specific antibody comprises a Chothia CDR-H1 sequence defined by the consensus sequence G-ε₁-T-ε₂-ε₃-ε₄-Y, wherein ε₁ is F or D; ε₂ is F or D; ε₃ is N or D; and ε₄ is K or D. In some embodiments, the bi-specific antibody comprises a Kabat CDR-H1 sequence defined by the consensus sequence μ₁-Y-μ₂-M-N, where μ₁ is K or D; μ₂ is A or D. In some embodiments, the bi-specific antibody comprises a Kabat CDR-H2 sequence defined by the consensus sequence θ₁-I-θ₃-θ₄-θ₅-θ₆-θ₇-N-Y-θ₁₀-T-θ₁₁-Y-A-D-S-V-K-D; wherein θ₁ is R or D; θ₃ is R or D; θ₄ is S or T; θ₅ is K or D; θ₆ is Y or D; θ₇ is N or D; θ₁₁ is A or D; and θ₁₁ is Y or D; and θ₁₁ is Y or D (SEQ ID NO: 295). In certain embodiments, θ₄ is T. In some embodiments, the bi-specific antibody comprises a Chothia CDR-H2 sequence defined by the consensus sequence ζ₁-ζ₂-ζ₃-ζ₄-ζ₅-N-Y-A, wherein ζ₁ is R or D; ζ₂ is S or T; ζ₃ is K or D; ζ₄ is Y or D; and (s is N or D. In certain embodiments, 42 is T.

In some embodiments, the bi-specific antibody or antigen binding construct comprises a first binding domain comprising a CDR-L1 sequence defined by the consensus sequence G-S-S-T-G-A-V-T-S-G-Y-Y-P-N (SEQ ID NO: 300). In some embodiments, the bi-specific antibody or antigen binding construct comprises a CDR-L2 sequence defined by the consensus sequence G-T-K-F-L-A-P (SEQ ID NO: 299) and selected from SEQ ID NOs: 159-181. In some embodiments, the bi-specific antibody or antigen binding fragment comprises a CDR-L3 sequence a sequence defined by the consensus sequence A-L-β₃-β₄-β₅-β₆-β₇-W-V (SEQ ID NO: 296), where β₃ is W or D; β₄ is Y, F, or D, β₅ is S or D, β₆ is N or D, and β₇ is R or D and selected from SEQ ID NOs: 183-205. In certain embodiments, β₇ is R.

In some embodiments, the bi-specific antibody or antigen binding construct comprises a first binding domain comprising a Chothia CDR-H3 sequence selected from SEQ ID NOs: 109-133. In some embodiments, the bi-specific antibody or antigen binding construct comprises a first binding domain comprises a Chothia CDR-H2 sequence selected from SEQ ID NOs: 57-81. In some embodiments, the bi-specific antibody or antigen binding construct comprises a first binding domain comprising a Chothia CDR-H1 sequence selected from SEQ ID NOs: 5-29. In some embodiments, the bi-specific antibody or antigen binding construct comprises a first binding first binding domain comprising a Kabat CDR-H2 sequence selected from SEQ ID NOs: 83-107. In some embodiments, the bi-specific antibody or antigen binding construct comprises a the first binding domain comprising a Kabat CDR-H1 sequence selected from SEQ ID NOs: 31-55.

In some embodiments, the bi-specific antibody or antigen binding construct comprises a first binding domain comprising a CDR-H3, a CDR-H2, CDR-H1, a CDR-L3, a CDR-L2, and a CDR-L1, each consisting of a CDR sequence of a V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some embodiments, the bi-specific antibody or antigen binding construct comprises a first binding domain comprising a CDR-H3, a CDR-H2, CDR-H1, a CDR-L3, a CDR-L2, a CDR-L1, and framework regions each consisting of a CDR sequence or framework sequence of a V_{H}-V_{L} pair selected from the group consisting of SEQ ID NOs: 211 and 237; 211 and 236; 211 and 235; 211 and 234; 211 and 233; 210 and 237; 210 and 236; 210 and 235; 210 and 234; 210 and 233; 209 and 237; 209 and 236; 209 and 235; 209 and 234; 209 and 233; 208 and 237; 208 and 236; 208 and 235; 208 and 234; 208 and 233; 207 and 237; 207 and 236; 207 and 235; 207 and 234; 207 and 233; 213 and 240; 213 and 239; 213 and 238; 212 and 240; 214 and 240; 215 and 240; 216 and 240; 217 and 240; 218 and 240; 220 and 240; 221 and 240; 222 and 240; 223 and 240; 224 and 240; 225 and 240; 226 and 240; 227 and 240; 228 and 240; 229 and 240; 230 and 240; 231 and 240; 212 and 241; 212 and 242; 212 and 243; 212 and 244; 212 and 245; 208 and 255; 208 and 254; 208 and 253; 208 and 252; 208 and 251; 208 and 250; 208 and 249; 208 and 248; 208 and 247; 208 and 246; 219 and 241; 216 and 241; 214 and 241; 211 and 241; 226 and 241; 219 and 241; 216 and 241; 214 and 241; 211 and 241; 226 and 241; 219 and 251; 216 and 251; 214 and 251; 211 and 251; 226 and 251; 220 and 250; 216 and 250; 214 and 250; 211 and 250; and 226 and 250.

In some embodiments, the bi-specific antibody comprises a first binding domain binds to CD3 and the second binding domain binds to epithelial cell adhesion molecule (EpCAM).

In some embodiments, the bi-specific antibody is humanized or human.

### EXAMPLES

### Example 1

### Humanization of Antibodies

The SP34 mouse monoclonal antibody (Silvana Pessano et al. EMBO J 1985, 4, 337-344) sequence was used as a starting point for humanization since it shows human and cyno cross-reactivity. The CDRs from the SP34 heavy chain were grafted onto human antibody frameworks VH3-15, VH3-23, VH3-49, and VH3-73 by standard methodology (Kuramochi et al. Methods in Molecular Biology 2014, 1060, 123-137) to yield humanized heavy chains, hSP34-HC1, hSP34-HC2, hSP34-HC3, and hSP34-HC4, respectively. All heavy chains used human IgG1 constant domains. The CDRs from the SP34 light chain were grafted onto human antibody frameworks Vk3-20, Vl2-11, Vl7-43, and Vl8-61 by standard methodology (Kuramochi et al. Methods in Molecular Biology 2014, 1060, 123-137) to yield humanized light chains, hSP34-LC1, hSP34-LC2, hSP34-LC3, and hSP34-LC4, respectively. LC1 uses a human kappa constant domain, while LC2, LC3, and LC4 use human lambda constant domains.

### Example 2

### Ribosome Display Selections

Individual SMP1913-F09 (hSP34-HC1+hSP34-LC3) DNA templates in either HC, LC, or scFv format were constructed by ATUM/DNA 2.0 suitable for constructing ribosome display libraries. Antibody libraries were constructed using a standard overlap extension PCR protocol (Heckman and Pease, Nat Protoc. 2007, 2, 924-932) with mutagenic primers targeting complementary determining regions (CDRs). For the scFv library, CDRs H1, H2, H3 and L3 were targeted. For the Fab library CDRs H1, H2, and H3 were targeted. Then scFv- (Hanes and Pluckthun Proc Natl Acad Sci USA 1997, 94, 4937-4942) and Fab-based (Stafford R L et al Protein Eng Des Sel, 2014, 4, 97-109) ribosome display selections were performed according to published protocols (Dreier and Pluckthun Method Mol Biol 2011, 687, 283-306) against a biotinylated CD3e protein (Acro biosystems). After multiple rounds of selection, the DNA from RT-PCR output was cloned into an optimized vector for cell-free expression (Yin et al. mAbs 2012, 4, 217-225) using standard molecular biology techniques. All constructs were HIS- and FLAG-tagged to streamline purification and testing during screening. This led to antibodies in the 2037 sequence family.

### Example 3

### 2037-B10 Stability/Affinity Engineering

Following the ribosome display protocol described above, the 2037-B10 antibody was further engineered to improve its stability, solubility, and optimize its affinity. In particular, it has been noted from the literature that lower affinity CD3 antibodies may be better tolerated in the context of tumor-targeting bispecific antibodies (e.g. Leong et al. Blood 2017, 129, 609-618; Moore et al Blood 2015 126: 1798; Saber et al. Regul Toxicol Pharmacol 2017, 90, 144-152).

First, the 2037-B10 CDRs were grafted onto human kappa frameworks Vk1-39 in a similar manner as described previously (Lehmann et al., mAbs 2015, 7, 6, 1058-1071) to generate 2037-B10-Lc1, 2037-B10-Lc2, 2037-B10-Lc3, 2037-B10-Lc4, 2037-B10-Lc5, 2037-B10-Lc6, 2037-B10-Lc7, 2037-B10-Lc8, 2037-B10-Lc9, and 2037-B10-Lc10. All light chains use human kappa constant domains. To evaluate solubility and aggregation resistance, aspartic acid mutations were initially introduced into 2037-B10 heavy chain around and in CDR H1 (F27D, F29D, N30D, K31D, A33D). Dudgeon et al. PNAS 2012, 109, 27, 10879-10884. Additional aspartic acid mutations were also designed around CDR H3 and L3, to evaluate aggregation resistance.

### Example 4

### Primary Screening of Antibody Variants

Libraries of antibody variants generated by selection workflow were transformed into E. coli and grown on agar plates with the antibiotic (Kanamycin). Individual colonies were grown in liquid broth (TB+antibiotic Kanamycin) and used as a template for DNA amplification via rolling circle amplification (RCA). The variants were then expressed in a cell-free protein synthesis reaction as described (Yin, G. et al. 2012). Briefly, cell-free extracts were treated with 50 μM iodoacetamide for 30 min at RT (20° C.) and added to a premix containing cell-free components (Cai, Q. et al. 2015), 100% (v/v) RCA DNA template (approximately 10 μg/mL DNA) for scFvFc variants of interest. 60 μL cell free (CF) reactions were incubated at 30° C. for 12 hr on a shaker at 650 rpm in 96-well plates. 400-1500 colonies were screened, depending on the predicted diversity of different selection campaigns.

Exemplary humanized and affinity-matured antibodies are reported in Tables 5-9, below.

TABLE 5
Humanized 1913 Antibodies

| | | Sequence ID: | | Sequence ID: |
|---|---|---|---|---|
| Variant ID | Heavy chain | V_{H}/IgG1 | Light chain | V_{L}/ [symbol] |
| SMP1913- | SP34-HC-VH + | 211/256 | SP34-VL + | 237/258 |
| F01 | human IgG1 | | human lambda | |
| SMP1913- | SP34-HC-VH + | 211/256 | hSP34-Lc1-VL + | 236/257 |
| F02 | human IgG1 | | human kappa | |
| SMP1913- | SP34-HC-VH + | 211/256 | hSP34-Lc2-VL + | 235/258 |
| F03 | human IgG1 | | human lambda | |
| SMP1913- | SP34-HC-VH + | 211/256 | hSP34-Lc3-VL + | 234/258 |
| F04 | human IgG1 | | human lambda | |
| SMP1913- | SP34-HC-VH + | 211/256 | hSP34-Lc4-VL + | 233/258 |
| F05 | human IgG1 | | human lambda | |
| SMP1913- | hSP34-Hc1-VH + | 210/256 | SP34-VL + | 237/258 |
| F06 | human IgG1 | | human lambda | |
| SMP1913- | hSP34-Hc1-VH + | 210/256 | hSP34-Lc1-VL + | 236/257 |
| F07 | human IgG1 | | human kappa | |
| SMP1913- | hSP34-Hc1-VH + | 210/256 | hSP34-Lc2-VL + | 235/258 |
| F08 | human IgG1 | | human lambda | |
| SMP1913- | hSP34-Hc1-VH + | 210/256 | hSP34-Lc3-VL + | 234/258 |
| F09 | human IgG1 | | human lambda | |
| SMP1913- | hSP34-Hc1-VH + | 210/256 | hSP34-Lc4-VL + | 233/258 |
| F10 | human IgG1 | | human lambda | |
| SMP1913- | hSP34-Hc2-VH + | 209/256 | SP34-VL + | 237/258 |
| F11 | human IgG1 | | human lambda | |
| SMP1913- | hSP34-Hc2-VH + | 209/256 | hSP34-Lc1-VL + | 236/257 |
| G01 | human IgG1 | | human kappa | |
| SMP1913- | hSP34-Hc2-VH + | 209/256 | hSP34-Lc2-VL + | 235/258 |
| G02 | human IgG1 | | human lambda | |
| SMP1913- | hSP34-Hc2-VH + | 209/256 | hSP34-Lc3-VL + | 234/258 |
| G03 | human IgG1 | | human lambda | |
| SMP1913- | hSP34-Hc2-VH + | 209/256 | hSP34-Lc4-VL + | 233/258 |
| G04 | human IgG1 | | human lambda | |
| SMP1913- | hSP34-Hc3-VH + | 208/256 | SP34-VL + | 237/258 |
| G05 | human IgG1 | | human lambda | |
| SMP1913- | hSP34-Hc3-VH + | 208/256 | hSP34-Lc1-VL + | 236/257 |
| G06 | human IgG1 | | human kappa | |
| SMP1913- | hSP34-Hc3-VH + | 208/256 | hSP34-Lc2-VL + | 235/258 |
| G07 | human IgG1 | | human lambda | |
| SMP1913- | hSP34-Hc3-VH + | 208/256 | hSP34-Lc3-VL + | 234/258 |
| G08 | human IgG1 | | human lambda | |
| SMP1913- | hSP34-Hc3-VH + | 208/256 | hSP34-Lc4-VL + | 233/258 |
| G09 | human IgG1 | | human lambda | |
| SMP1913- | hSP34-Hc4-VH + | 207/256 | SP34-VL + | 236/258 |
| G10 | human IgG1 | | human lambda | |
| SMP1913- | hSP34-Hc4-VH + | 207/256 | hSP34-Lc1-VL + | 236/257 |
| G11 | human IgG1 | | human kappa | |
| SMP1913- | hSP34-Hc4-VH + | 207/256 | hSP34-Lc2-VL + | 235/258 |
| H01 | human IgG1 | | human lambda | |
| SMP1913- | hSP34-Hc4-VH + | 207/256 | hSP34-Lc3-VL + | 234/258 |
| H02 | human IgG1 | | human lambda | |
| SMP1913- | hSP34-Hc4-VH + | 207/256 | hSP34-Lc4-VL + | 233/258 |
| H03 | human IgG1 | | human lambda | |

TABLE 6
Affinity Matured 2037 Antibodies

| | | Sequence ID: | | Sequence ID: |
|---|---|---|---|---|
| Variant ID | Heavy chain | V_{H}/IgG1 | Light chain | V_{L}/ [symbol] |
| SRP2037-A02 | B02/A11/A02-VH + | 213/256 | 2037-A02/B10-VL + | 240/258 |
| | human IgG1 | | human lambda | |
| SRP2037-A11 | B02/A11/A02-VH + | 213/256 | 2037-A11-VL + | 239/258 |
| | human IgG1 | | human lambda | |
| SRP2037-B02 | B02/A11/A02-VH + | 213/256 | 2037-B02-VL + | 238/258 |
| | human IgG1 | | human lambda | |
| SRP2037-B10 | 2037-B10-VH + | 212/256 | 2037-A02/B10-VL + | 240/258 |
| | human IgG1 | | human lambda | |

TABLE 7
Affinity Matured 2037 Antibodies with Single Mutations

| Variant ID | | Sequence ID: | | Sequence ID: |
|---|---|---|---|---|
| SRP 2037 | Heavy chain | V_{H}/IgG1 | Light chain | V_{L}/ [symbol] |
| Y180D | 2037-B10_VH-Y108D + | 214/256 | 2037-A02/B10-VL + | 240/258 |
| | human IgG1 | | human lambda | |
| S107D | 2037-B10_VH-S107D + | 215/256 | 2037-A02/B10-VL + | 240/258 |
| | human IgG1 | | human lambda | |
| N106D | 2037-B10_VH-N106D + | 216/256 | 2037-A02/B10-VL + | 240/258 |
| | human IgG1 | | human lambda | |
| G105D | 2037-B10_VH-G105D + | 217/256 | 2037-A02/B10-VL + | 240/258 |
| | human IgG1 | | human lambda | |
| F104D | 2037-B10_VH-F104D + | 218/256 | 2037-A02/B10-VL + | 240/258 |
| | human IgG1 | | human lambda | |
| N103D | 2037-B10_VH-N103D + | 219/256 | 2037-A02/B10-VL + | 240/258 |
| | human IgG1 | | human lambda | |
| Y61D | 2037-B10_VH-Y61D + | 220/256 | 2037-A02/B10-VL + | 240/258 |
| | human IgG1 | | human lambda | |
| A59D | 2037-B10_VH-A59D + | 221/256 | 2037-A02/B10-VL + | 240/258 |
| | human IgG1 | | human lambda | |
| N56D | 2037-B10_VH-N56D + | 222/256 | 2037-A02/B10-VL + | 240/258 |
| | human IgG1 | | human lambda | |
| Y55D | 2037-B10_VH-Y55D + | 223/256 | 2037-A02/B10-VL + | 240/258 |
| | human IgG1 | | human lambda | |
| K54D | 2037-B10_VH-K54D + | 224/256 | 2037-A02/B10-VL + | 240/258 |
| | human IgG1 | | human lambda | |
| R52D | 2037-B10_VH-R52D + | 225/256 | 2037-A02/B10-VL + | 240/258 |
| | human IgG1 | | human lambda | |
| R50D | 2037-B10_VH-R50D + | 226/256 | 2037-A02/B10-VL + | 240/258 |
| | human IgG1 | | human lambda | |
| A33D | 2037-B10_VH-A33D + | 227/256 | 2037-A02/B10-VL + | 240/258 |
| | human IgG1 | | human lambda | |
| K31D | 2037-B10_VH-K31D + | 228/256 | 2037-A02/B10-VL + | 240/258 |
| | human IgG1 | | human lambda | |
| N30D | 2037-B10_VH-N30D + | 229/256 | 2037-A02/B10-VL + | 240/258 |
| | human IgG1 | | human lambda | |
| F29D | 2037-B10_VH-F29D + | 230/256 | 2037-A02/B10-VL + | 240/258 |
| | human IgG1 | | human lambda | |
| F27D | 2037-B10_VH-F27D + | 231/256 | 2037-A02/B10-VL + | 240/258 |
| | human IgG1 | | human lambda | |
| R97D | 2037-B10-VH + | 212/256 | 2037-B10 VL-R97D + | 241/258 |
| | human IgG1 | | human lambda | |
| N96D | 2037-B10-VH + | 212/256 | 2037-B10 VL-N96D + | 242/258 |
| | human IgG1 | | human lambda | |
| S95D | 2037-B10-VH + | 212/256 | 2037-B10 VL-S95D + | 243/258 |
| | human IgG1 | | human lambda | |
| Y94D | 2037-B10-VH + | 212/256 | 2037-B10 VL-Y94D + | 244/258 |
| | human IgG1 | | human lambda | |
| W93D | 2037-B10-VH + | 212/256 | 2037-B10 VL-W93D + | 245/258 |
| | human IgG1 | | human lambda | |

TABLE 8
Affinity Matured 2037-B10 Kappa Grafts

| | | Sequence ID: | | Sequence ID: |
|---|---|---|---|---|
| Variant ID | Heavy chain | V_{H}/IgG1 | Light chain | V_{L}/_{κ} |
| 2037-B10-Lc1 | 2037-B10-VH + | 212/256 | 2037-B10-Lc1-VL + | 255/257 |
| | human IgG1 | | human kappa | |
| 2037-B10-Lc2 | 2037-B10-VH + | 212/256 | 2037-B10-Lc2-VL + | 254/257 |
| | human IgG1 | | human kappa | |
| 2037-B10-Lc3 | 2037-B10-VH + | 212/256 | 2037-B10-Lc3-VL + | 253/257 |
| | human IgG1 | | human kappa | |
| 2037-B10-Lc4 | 2037-B10-VH + | 212/256 | 2037-B10-Lc4-VL + | 252/257 |
| | human IgG1 | | human kappa | |
| 2037-B10-Lc5 | 2037-B10-VH + | 212/256 | 2037-B10-Lc5-VL + | 251/257 |
| | human IgG1 | | human kappa | |
| 2037-B10-Lc6 | 2037-B10-VH + | 212/256 | 2037-B10-Lc6-VL + | 250/257 |
| | human IgG1 | | human kappa | |
| 2037-B10-Lc7 | 2037-B10-VH + | 212/256 | 2037-B10-Lc7-VL + | 249/257 |
| | human IgG1 | | human kappa | |
| 2037-B10-Lc8 | 2037-B10-VH + | 212/256 | 2037-B10-Lc8-VL + | 248/257 |
| | human IgG1 | | human kappa | |
| 2037-B10-Lc9 | 2037-B10-VH + | 212/256 | 2037-B10-Lc9-VL + | 247/257 |
| | human IgG1 | | human kappa | |
| 2037-B10-Lc10 | 2037-B10-VH + | 212/256 | 2037-B10-Lc10-VL + | 246/257 |
| | human IgG1 | | human kappa | |

TABLE 9
Affinity Matured 2037-B10 Combination Asp + Kappa Grafts

| Variant ID | | Sequence ID: | | Sequence ID: |
|---|---|---|---|---|
| SRP 2037-B10 | Heavy chain | V_{H}/IgG1 | Light chain | V_{L}/ [symbol] |
| Y55D/R97D | 2037-B10_VH-Y55D + | 223/256 | 2037-B10 VL-R97D + | 241/258 |
| | human IgG1 | | human lambda | |
| Y61D/R97D | 2037-B10_VH-Y61D + | 220/256 | 2037-B10 VL-R97D + | 241/258 |
| | human IgG1 | | human lambda | |
| F104D/R97D | 2037-B10_VH-F104D + | 218/256 | 2037-B10 VL-R97D + | 241/258 |
| | human IgG1 | | human lambda | |
| S107D/R97D | 2037-B10_VH-S107D + | 215/256 | 2037-B10 VL-R97D + | 241/258 |
| | human IgG1 | | human lambda | |
| F29D/R97D | 2037-B10_VH-F29D + | 230/256 | 2037-B10 VL-R97D + | 241/258 |
| | human IgG1 | | human lambda | |
| Y55D/LC5 | 2037-B10_VH-Y55D + | 223/256 | 2037-B10-Lc5-VL + | 251/257 |
| | human IgG1 | | human kappa | |
| Y61D/LC5 | 2037-B10_VH-Y61D + | 220/256 | 2037-B10-Lc5-VL + | 251/257 |
| | human IgG1 | | human kappa | |
| F104D/LC5 | 2037-B10_VH-F104D + | 218/256 | 2037-B10-Lc5-VL + | 251/257 |
| | human IgG1 | | human kappa | |
| S107D/LC5 | 2037-B10_VH-S107D + | 215/256 | 2037-B10-Lc5-VL + | 251/257 |
| | human IgG1 | | human kappa | |
| F29D/LC5 | 2037-B10_VH-F29D + | 230/256 | 2037-B10-Lc5-VL + | 251/257 |
| | human IgG1 | | human kappa | |
| Y55D/LC6 | 2037-B10_VH-Y55D + | 223/256 | 2037-B10-Lc6-VL + | 250/257 |
| | human IgG1 | | human kappa | |
| Y61D/LC6 | 2037-B10_VH-Y61D + | 220/256 | 2037-B10-Lc6-VL + | 250/257 |
| | human IgG1 | | human kappa | |
| F104D/LC6 | 2037-B10_VH-F104D + | 218/256 | 2037-B10-Lc6-VL + | 250/257 |
| | human IgG1 | | human kappa | |
| S107D/LC6 | 2037-B10_VH-S107D + | 215/256 | 2037-B10-Lc6-VL + | 250/257 |
| | human IgG1 | | human kappa | |
| F29D/LC6 | 2037-B10_VH-F29D + | 230/256 | 2037-B10-Lc6-VL + | 250/257 |
| | human IgG1 | | human kappa | |

### Example 5

### ELISA Characterization

Following small-scale cell-free protein synthesis (60 μL reactions), each reaction was diluted 1:50 and tested for binding to CD3 protein by ELISA. Briefly, human CD3 delta-epsilon-Fc heterodimer (CD3DE-Fc, Acro Biosystems, Cat #CDD-H52WO), cynomolgus CD3 delta-epsilon heterodimer (CD3DE, Acro Cat #CDD-C52W4) and human IgG1 Fc (Acro Biosystems, Cat #FCC-H5214) were coated to 384-well Maxisorp plates in 0.2M sodium bicarbonate-carbonate pH 9.4 and blocked with 200 BSA in PBST (phosphate buffer saline, 0.100 Tween20, diluted from Teknova Cat #P2192). Antibodies expressed in CF were diluted 1:50 in 0.20% BSA in PBST and were incubated on the CD33-coated plates, washed, and detected with HRP-conjugated anti-human Fab antibodies (Jackson ImmunoResearch, West Grove, PA) and Pierce Pico Supersignal ELISA substrate (ThermoFisher Scientific).

For ELISA characterization of purified antibodies, antibody variants were normalized to 200-500 nM and then serially diluted in 0.200 BSA in PBST before being applied to CD3-coated plates as described above. Binding was detected with HRP-conjugated anti-human Fab antibodies and Pierce Pico Supersignal ELISA substrate. ELISA binding was plotted as a function of antibody concentration and fitted using non-linear regression analysis using Softmax Pro. The EC50 (the antibody concentration at the midpoint of the binding curve) and Bₘax (maximal binding signal at saturating concentrations of antibody) were reported.

### Example 6

### Cell Lines and Cell Culture Conditions

Jurkat, Jurkat RT3-T3.5 and HUT78 cells, were obtained from ATCC and were maintained in RPMI-1640 (Cellgro-Mediatech) supplemented with 20% heat-inactivated fetal bovine serum (Sigma), 1% Penicillin/Streptomycin (Cellgro-Mediatech), and 2 mmol/L-glutamax (Life Technology).

NCI-H1975 cells were obtained from ATCC and were maintained in DMEM/F12 (Cellgro-Mediatech) supplemented with 10% heat-inactivated fetal bovine serum (Sigma), 1% Penicillin/Streptomycin (Cellgro-Mediatech), and 2 mmol/L-glutamax (Life Technology).

### Example 7

### High-Throughput Cell Binding

A high-throughput primary screen was performed to rapidly assess cell binding of antibodies produced in small-scale (60 μL) cell-free reactions. In this screen, four components are combined in equal volumes to a final volume of 100 μL/well in a U-bottom 96-well plate (Greiner Cat #650201) or flat bottom 384 well plate (Greiner Cat #781201). These components are as follows: 1) CD3-expressing Jurkat cells diluted in assay buffer (1×PBS+0.2% BSA, sterile filtered) to achieve a final concentration of 50,000 cells/well, 2) CD3-negative Jurkat RT3-T3.5 cells stained with CellTrace Oregon Green (Invitrogen Cat #34555) and diluted in assay buffer to achieve a final concentration of 50,000 cells/well, 3) a 1:50 dilution of cell-free reaction producing the antibody of interest diluted in assay buffer, and 4) a secondary anti-human antibody (AlexaFluor 647 AffiniPure F(ab′)₂ Donkey anti-human IgG, Fc specific; Jackson ImmunoResearch Cat #709-606-098) diluted 1:100 in assay buffer. Plates are then incubated on ice for one hour. Cells are pelleted by spinning at 1500×g for 5 minutes and resuspended in assay buffer. High-throughput flow cytometry is then performed on resuspended cells on a FACS instrument (BD Biosciences FACS Canto II or BD Biosciences LSR II) and data is analyzed with FlowJo software. Antibody binding is assessed by the proportional level of secondary antibody signal (presumably due to binding to our antibody of interest) on Jurkat CD3-positive cells compared to the signal on Jurkat RT3.T3.5 CD3-negative cells.

### Example 8

### Small-Scale Purification of Antibody Variants

The top leads from an initial round of screening were cultured and miniprepped via the Qiaprep 96 Turbo miniprep kit (Qiagen) according to manufacturer's instructions. 10 μg/mL miniprepped scFvFc DNA was added to 4 mL cell-free reactions and incubated overnight for 12 hr at 25-30° C., 650 rpm. Other variants for humanization, affinity-modulation, or TAG-scan variants with non-natural amino acid incorporation were similarly expressed from maxiprep plasmid at an optimized HC:LC plasmid ratio (typically 3:1). Expressed variants from clarified cell-free reactions were purified via IMAC purification using a semi-automated high throughput batch purification method. Briefly, purifications were performed in a 96-well plate format where 50 L/well of IMAC resin (Ni Sepharose High Performance, GE Healthcare) was equilibrated in IMAC binding buffer (50 mM HEPES pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM HEPES pH 8.0, 300 mM NaCl, 500 mM imidazole). For some samples, a CH1-XL affinity matrix (ThermoFisher Scientific, Cat #2943452010) was utilized to achieve higher purity samples. Briefly, cell-free reactions were applied to a CH1-XL resin that was prewashed with 20 mM Tris pH 7.5 and incubated for 15-30 minutes to allow for antibody binding. The resin was subsequently washed with 20 mM Tris pH 7.5, and purified antibodies were eluted with 20 mM citric acid, 150 mM NaCl, pH 3.5 followed by neutralization by adding 1 M Tris-HCl pH 9. All samples were buffer exchanged into PBS+10% sucrose using a 96-well Zeba plate (7 kDa MWCO, Thermofisher) and quantified via high throughput capillary electrophoresis using the Labchip GXII (Perkin Elmer) against a Herceptin standard curve, per manufacturer's instructions.

### Example 9

### Differential Scanning Fluorimetry

A protein thermal shift assay was carried out by mixing the protein to be assayed with an environmentally sensitive dye (SYPRO Orange, Life Technologies Cat #S-6650) in a phosphate buffered solution (PBS), and monitoring the fluorescence of the mixture in real time as it underwent controlled thermal denaturation. Protein solutions between 0.2-2 mg/mL were mixed at a 1:1 volumetric ratio with a 1:250 PBS-diluted solution of SYPRO Orange (SYPRO Orange stock dye is 5000× in DMSO). 5 μL aliquots of the protein-dye mixture were dispensed in quadruplicate in a 384-well microplate that was pretreated for 30 minutes at 95° C. (Bio-Rad Cat #MSP-3852), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (˜1.5° C. per minute), allowing 3 seconds of equilibration at each temperature before taking a fluorescence measurement. At the end of the experiment, the transition melting temperature (Fab™) was determined using the Bio-Rad CFX manager software.

### Example 10

### Biacore Off-Rate and Kinetic Analysis

Anti-Fab or anti-Fc polyclonal antibodies were immobilized onto a CM5 chip (GE Life Sciences) using amine coupling chemistry (from Amine Coupling Kit, GE Life Sciences). The immobilization steps were carried out at a flow rate of 25 μl/min in 1×HBS-EP+ buffer (GE Life Sciences; 10× Stock diluted before use). The sensor surfaces were activated for 7 min with a mixture of NHS (0.05 M) and EDC (0.2 M). The Anti-Fab or Anti-Fc antibodies were injected over all 4 flow cells at a concentration of 25 μg/mL in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and kinetic binding experiments were performed at 25° C. using 1×HBS-EP+ buffer. Test and control antibodies were injected over the anti-Fab or anti-Fc surface at concentrations of 5-10 μg/mL for 12 seconds at a flow rate of 10 μL/min on flow cells 2, 3 and 4, followed by a buffer wash for 30 seconds at the same flow rate. Kinetic characterization of antibody samples was carried out with a range of antigen concentrations from 1-100 nM and one injection of 0 nM antigen (for example, 100, 50, 25, 6.25, 1.56 and 0 nM). After capturing ligand (antibody) on the anti-Fc or anti-Fab surface, the analyte (human CD3 epsilon, Acro Cat #CDE-H5223) was bound for 180 seconds, followed by a 600 second dissociation phase at a flow rate of 50 μL/min. Between each ligand capture and analyte binding cycle, regeneration was carried out using two injections of 10 mM Glycine pH 2.0 for 30 seconds at 30 μL/min, followed by a 30 second buffer wash step.

The data was fit with the Biacore T200 Evaluation software, using a 1-1 Langmuir binding model. K_{D} (affinity, nM) was determined as a ratio of the kinetic rate constants calculated from the fits of the association and dissociation phases.

### Example 11

### Preparation of Bi-Specific Antibodies

The bispecific antibodies were generated as tandem scFv's with an anti-EpCAM scFv followed by an anti-CD3 scFv and joined by a short GGGGS linker (SEQ ID NO: 308). Specifically, the anti-EpCAM scFv was constructed in a VL to VH orientation whereas the anti-CD3 scFv was constructed in a VH to VL orientation. VH and VL domains were joined by a standard linker familiar to those in the art.

### Example 12

### Re-Directed T Cell Killing Assay

PBMCs were isolated from human blood donors by Leukosep tube and Nycoprep 1.077 buffer according to the manufacture's recommendation. CD8+ T cells were isolated by immunomagnetic negative selection from fresh PBMCs (Miltenyi #130-096-495—human CD8+ T cell isolation kit). Isolated CD8+ T cells were cultured in primary T cell culture media (RPMI supplemented with 10% heat-inactivated fetal bovine serum from Hyclone, 1% Penicillin/Streptomycin and 2 mmol/L-glutamax) before the assay. The day before the assay, EpCam positive lung cancer cell NCI-H1975 cells were stained with Oregon green (Thermo Fisher Scientific) and plated on 96-well half area plates at 5 k cells per well in NCI-H1975 culture media (DMEM/F12 media with 10% FBS from Sigma, 1% Penicillin/Streptomycin and 2 mmol/L-glutamax). The next day, the cell culture media was replaced with 25 ul of primary T cell culture media. CD8+ T cells were added into each well at 5 k/cell in 25 ul primary T cell culture media. EpCAM-CD3 bispecific antibodies were also added into the well at 3× of the final concentration in 25 ul of primary T cell culture media. The plates were incubated in a cell culture incubator for 2 days before the cells were collected with Accutase (Cellgro-Mediatech). The cells were washed once and propidium iodide (1:200) were added to stain the dead cells before the cells were read on FACS Canto. The specific cell killing was reported as % of dead target cells/total target cells in each well and data was expressed as % specific cell killing vs. dose of bi-specific antibody in nM. The cell killing EC50 was reported by GraphPad Prism using non-linear regression analysis, using log(inhibitor) vs. response, variable slope, 4-parameter fit equations.

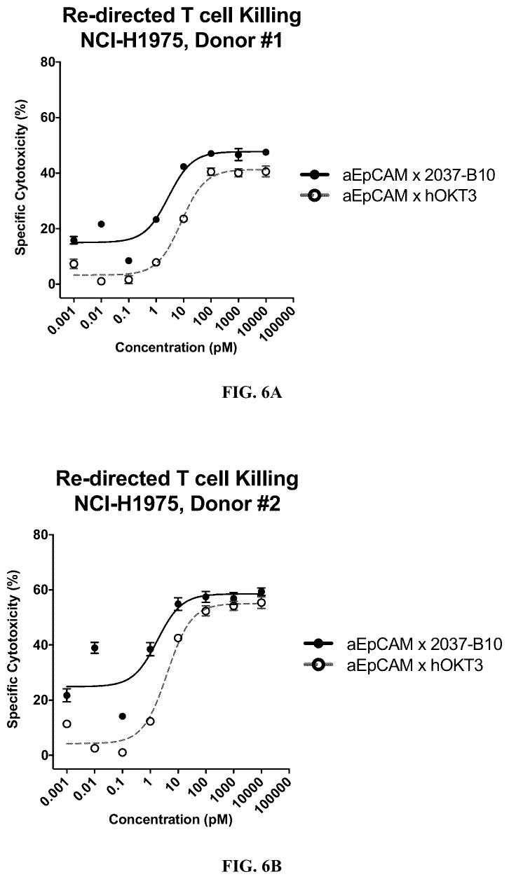

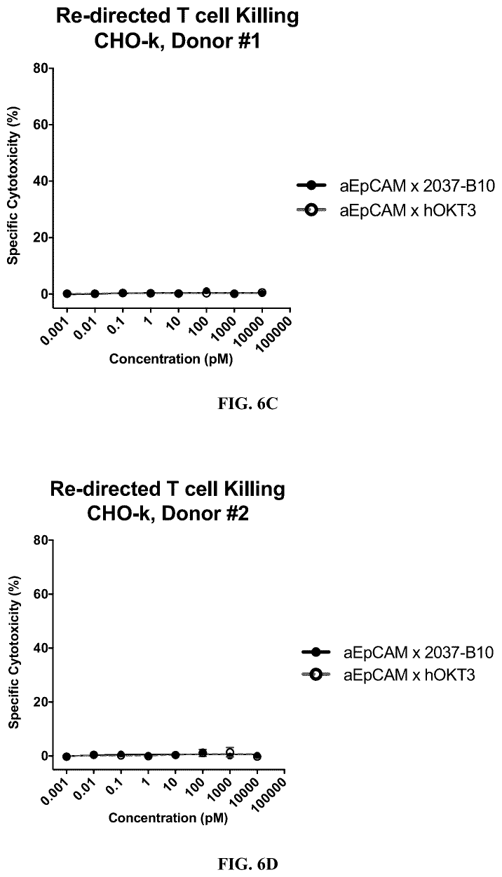

In the presence of human CD8+ T cells, EpCAM-CD3 showed potent cell killing activity on EpCAM positive NCI-H1975 cells, but not on the EpCAM negative CHO-k cell (FIG. 6A-6D and Table 10).

TABLE 10

| | | Cell killing | Cell killing |
|---|---|---|---|
| SEQ | | Donor #1 | Donor #2 |
| ID NO: | Sample | EC50 (pM) | EC50 (pM) |
| 262 | aEpCAM × hOKT3 | 8.2 | 4.1 |
| 263 | aEpCAM × 2037-B10 | 2.8 | 1.8 |

### Example 13

### T Cell Activation and Cytokine Release Assay

PBMCs were isolated from human blood donors by Leukosep tube and Nycoprep 1.077 buffer according to the manufacture's recommendation. CD4+ T cells were isolated by immunomagnetic negative selection from fresh PBMCs (Miltenyi #130-096-533—human CD4+ T cell isolation kit). Isolated CD4+ T cells were cultured in primary T cell culture media before the assay. The day before the assay, 25 ul of anti-CD3 antibodies at 5 ug/mL in PBS were coated onto 96-well half area plates at 4° C. overnight. The next day, the plates were washed twice with PBS and 50 k of CD4+ T cells were added into each well and incubated in a cell culture incubator for 48 hrs. IFN-g and IL-2 levels in the cell culture conditional media were measured by ELISA kits according to the manufacture recommendations (Human IFN-gamma Quantikine ELISA Kit and Human IL-2 Quantikine ELISA Kit from R&D system).

### Example 14

### PREPARATION OF scFvs

A single-chain antibody is made in either the V_{H}V_{L} or V_{L}V_{H} orientation with a linker sequence between the V_{H} and V_{L} domains. Typically, scFv linkers are composed of (GGGGS)n (SEQ ID NO: 309) repeats where n=3, 4, 5, or 6 for linkers of 15, 20, 25, or 30 residues respectively. For cell-free expression, an N-terminal Met is added, but for mammalian expression a leader peptide is added. On the C-terminal end of the scFv, an Fc sequence can be added to extend in vivo half-life or the scFv can be used directly. An optional linker sequence can be incorporated between the scFv and the Fc. An exemplary scFv-Fc linker sequence is AAGSDQEPKSS (SEQ ID NO: 291). C-terminal affinity tags can optionally be added to facilitate purification and assay development. An exemplary affinity tag is a C-terminal FlagHis tag GSGDYKDDDDKGSGHHHHHH (SEQ ID NO: 292). A stop codon is typically inserted at the end of the sequence. An exemplary scFv of the present disclosure is SEQ ID NO: 287, with an N-terminal Met residue, a V_{H} domain, a GGGGSGGGGSGGGGS (SEQ ID NO: 293) linker, a VL domain, an AAGSDQEPKSS (SEQ ID NO: 291) linker, an Fc domain, a FlagHis tag, and a stop codon.

### Example 15

### Characteristics of Illustrative Anti-CD3 Antibodies

Tables 11 through 20 show results obtained using the illustrative antibodies described herein.

Table 11 shows the results obtained with antibodies isolated from affinity maturation of initial the SMP 1913 antibody lead. Table 11 shows the estimated antibody concentration recovered from an expression extract. Table 11 shows unexpected results demonstrating that even though the antibodies were derived from a common lead antibody, the binding affinities to Jurkat cells and relative expression vary.

TABLE 11

| | | FACS S/N | estimated |
|---|---|---|---|
| | | (Jurkat/Jurkat | antibody |
| | humanized SP34 | RT3-T3.5 | recovered |
| Clone | derivative | binding) | (μg/mL) |
| SMP1913-F09 | hSP34-Hc1 + SP34-Lc3 | 6.2 | 598 |
| SMP1913-F04 | SP34-Hc + SP34-Lc3 | 5.1 | 707 |
| SMP1913-G10 | hSP34-Hc4 + SP34-Lc | 1.7 | 87 |
| SMP1913-F06 | hSP34-Hc1 + SP34-Lc | 1.6 | 409 |
| SMP1913-F01 | SP34-Hc + SP34-Lc | 1.5 | 184 |
| SMP1913-G02 | hSP34-Hc2 + SP34-Lc | 1.4 | 54 |
| SMP1913-G01 | hSP34-Hc2 + SP34-Lc3 | 1.3 | 185 |
| SMP1913-F10 | hSP34-Hc1 + SP34-Lc4 | 1 | 75 |
| SMP1913-G04 | hSP34-Hc2 + SP34-Lc4 | 1 | 41 |
| SMP1913-G08 | hSP34-Hc3 + SP34-Lc3 | 1 | 61 |
| SMP1913-H02 | hSP34-Hc4 + SP34-Lc3 | 1 | 63 |
| SMP1913-F03 | SP34-Hc + SP34-Lc2 | 1 | 78 |
| SMP1913-F08 | hSP34-Hc1 + SP34-Lc2 | 0.9 | 51 |
| SMP1913-G03 | hSP34-Hc2 + SP34-Lc2 | 0.9 | 31 |
| SMP1913-F02 | SP34-Hc + SP34-Lc1 | 0.9 | 106 |
| SMP1913-F05 | SP34-Hc + SP34-Lc4 | 0.9 | 93 |
| SMP1913-F07 | hSP34-Hc1 + SP34-Lc1 | 0.8 | 118 |
| SMP1913-F11 | hSP34-Hc2 + SP34-Lc1 | 0.8 | 36 |
| SMP1913-G05 | hSP34-Hc3 + SP34-Lc | 0.8 | 44 |
| SMP1913-G06 | hSP34-Hc3 + SP34-Lc1 | NT | ND |
| SMP1913-G07 | hSP34-Hc3 + SP34-Lc2 | NT | ND |
| SMP1913-G09 | hSP34-Hc3 + SP34-Lc4 | NT | ND |
| SMP1913-G11 | hSP34-Hc4 + SP34-Lc1 | NT | ND |
| SMP1913-H01 | hSP34-Hc4 + SP34-Lc2 | NT | ND |
| SMP1913-H03 | hSP34-Hc4 + SP34-Lc4 | NT | ND |

ND = not detected;
NT = not tested

TABLE 12
Table 12: A comparison of the Mouse SP34 antibody when compared to the generated antibody 1913-F09.
The results show that the SMP 1913-F09 antibody is active for binding to human and cynomolgus CD3.

| | humanized | ELISA EC₅₀ (nM) | | | | ELISA Bmax (RLU) | | | |
|---|---|---|---|---|---|---|---|---|---|
| | SP34 | no | CD3- | human | cyno | no | CD3- | human | cyno |
| Clone | derivative | CD3 | GE | CD3E | CD3E | CD3 | GE | CD3E | CD3E |
| Mouse | | 197 | 574 | 251 | 352 | 1.92E+03 | 3.61E+03 | 1.23E+05 | 1.16E+05 |
| SP34 | | | | | | | | | |
| SMP1913- | hSP34-Hc1 + | 971 | 1110 | 28.9 | 35.6 | 1.74E+06 | 1.70E+06 | 1.62E+06 | 1.57E+06 |
| F09 | SP34-Lc3 | | | | | | | | |

TABLE 13
Table 13: The characteristics of the SRP 2037 antibodies generated
by affinity maturation of the SMP1913-F09 antibody.

| | | Single point | | | | | | | |
|---|---|---|---|---|---|---|---|---|---|
| | | ELISA binding | | | | | | | |
| | | to CD3DE-Fc | | | | Cell binding, | | | |
| | | | huCD3DE- | Cell binding, | | Jurkat RT3- | | T cell | |
| SEQ | | cyCD3DE/Fc, | Fc/Fc, | Jurkat | | T3.5 | | activation | |
| ID | | ELISA | ELISA | | K_{d} | | K_{d} | IFN | IL2 |
| NO: | Clone | S/N | S/N | Bₘₐₓ | (nM) | Bₘₐₓ | (nM) | (ng/ml) | (ng/ml) |
| 264 | SRP | 76.8 | 59.0 | 5987 | 0.16 | 406 | 0.59 | 0.2 | 0.1 |
| | 2037:A01 | | | | | | | | |
| 265 | SRP | 102.8 | 80.6 | 6340 | 0.12 | 881 | 9.56 | 1.49 | 0.24 |
| | 2037:A02 | | | | | | | | |
| 266 | SRP | 62.1 | 55.1 | 4535 | 0.16 | 263 | 0.35 | 0 | 0.03 |
| | 2037:A03 | | | | | | | | |
| 267 | SRP | 19.3 | 14.7 | 4804 | 1.7 | NC | NC | 0.02 | 0.07 |
| | 2037:A04 | | | | | | | | |
| 268 | SRP | 28.9 | 21.2 | 5896 | 0.12 | 425 | 0.73 | 0.96 | 0.27 |
| | 2037:A05 | | | | | | | | |
| 269 | SRP | 64.9 | 47.0 | 5824 | 0.1 | NC | NC | 1.04 | 0.24 |
| | 2037:A06 | | | | | | | | |
| 27 | SRP | 103.5 | 89.9 | 4771 | 0.14 | 429 | 3.28 | 1.22 | 0.31 |
| | 2037:A08 | | | | | | | | |
| 272 | SRP | 48.6 | 41 | 4874 | 0.1 | 456 | 2.56 | 1.07 | 0.24 |
| | 2037:A09 | | | | | | | | |
| 273 | SRP | 24.2 | 59.7 | 4876 | 0.13 | 336 | 0.87 | 2.21 | 0.35 |
| | 2037:A11 | | | | | | | | |
| 275 | SRP | 82.9 | 73.4 | 5946 | 0.09 | 716 | 7.16 | 1.84 | 0.33 |
| | 2037:B02 | | | | | | | | |
| 276 | SRP | 44.0 | 31.1 | 6271 | 1.68 | 501 | 5.12 | 0 | 0.04 |
| | 2037:B03 | | | | | | | | |
| 277 | SRP | 172.6 | 131.1 | 5952 | 0.59 | 625 | 6.35 | 4.41 | 0.98 |
| | 2037:B04 | | | | | | | | |
| 278 | SRP | 120.7 | 93.5 | 6130 | 0.13 | 445 | 0.5 | 0.16 | 0.07 |
| | 2037:B05 | | | | | | | | |
| 279 | SRP | 208.5 | 162.1 | 5042 | 0.17 | NC | NC | 0 | 0.02 |
| | 2037:B06 | | | | | | | | |
| 280 | SRP | 43.5 | 35.9 | 5751 | 0.17 | 350 | 0.45 | 1.99 | 0.44 |
| | 2037:B07 | | | | | | | | |
| 281 | SRP | 200.7 | 161 | 4819 | 0.34 | 492 | 7.14 | 0.27 | 0.14 |
| | 2037:B08 | | | | | | | | |
| 282 | SRP | 63.5 | 47.2 | 5361 | 0.08 | 326 | 0.13 | 0.45 | 0.17 |
| | 2037:B09 | | | | | | | | |
| 283 | SRP | 203.9 | 144.4 | 4913 | 0.18 | 528 | 7.59 | 3.73 | 0.45 |
| | 2037:B10 | | | | | | | | |
| 284 | SRP | 64.0 | 46.2 | 5057 | 0.16 | 351 | 1.25 | 1.89 | 0.33 |
| | 2037:B11 | | | | | | | | |
| 285 | SRP | 136.4 | 107.5 | 6233 | 0.19 | 496 | 1.26 | 3.04 | 0.41 |
| | 2037:C01 | | | | | | | | |
| 286 | SRP | 80.2 | 61.7 | 6646 | 0.16 | NC | NC | 1.92 | 0.35 |
| | 2037:C02 | | | | | | | | |
| 287 | SRP | 67.4 | 44.1 | 6132 | 2.3 | 814 | 9.2 | 0 | 0.04 |
| | 2037:C03 | | | | | | | | |
| 288 | SRP | 114.2 | 88.1 | 5366 | 0.34 | 673 | 7.4 | 0.14 | 0.13 |
| | 2037:C04 | | | | | | | | |
| 289 | SRP | 177.1 | 130.5 | 6826 | 0.22 | 875 | 7.46 | 1.98 | 0.32 |
| | 2037:C05 | | | | | | | | |
| 290 | SRP | 99.7 | 77.5 | 6971 | 0.19 | NC | NC | 1.46 | 0.26 |
| | 2037:C06 | | | | | | | | |

NC = not calculable due to incomplete titration

TABLE 14
Table 14: The characteristics of the SRP 2037 antibodies generated
by affinity maturation of the SMP1913-F09 using the Biacore assay.

| SEQ | | | | | |
|---|---|---|---|---|---|
| ID | Biacore ligand | Biacore | kd₁ | K_{D} | Chi²/ |
| NO: | (scFvFc) | analyte | (1/s) | (M) | Rₘₐₓ |
| 264 | SRP2037:A01 | rhCD3E | 4.98E−04 | 2.36E−09 | 1.10% |
| 265 | SRP2037:A02 | rhCD3E | 1.71E−06 | 6.58E−12 | 1.20% |
| 266 | SRP2037:A03 | rhCD3E | 2.35E−04 | 1.23E−09 | 0.80% |
| 267 | SRP2037:A04 | rhCD3E | 3.27E−06 | 3.67E−10 | 3.50% |
| 268 | SRP2037:A05 | rhCD3E | 1.57E−04 | 7.12E−10 | 1.60% |
| 269 | SRP2037:A06 | rhCD3E | 3.47E−04 | 1.21E−09 | 1.90% |
| 271 | SRP2037:A08 | rhCD3E | 2.37E−04 | 1.21E−09 | 1.50% |
| 272 | SRP2037:A09 | rhCD3E | 1.79E−04 | 7.47E−10 | 1.20% |
| 273 | SRP2037:A11 | rhCD3E | 1.35E−04 | 5.93E−10 | 1.40% |
| 275 | SRP2037:B02 | rhCD3E | 4.67E−04 | 2.54E−09 | 0.90% |
| 276 | SRP2037:B03 | rhCD3E | 3.37E−03 | 1.24E−06 | 0.40% |
| 277 | SRP2037:B04 | rhCD3E | 1.01E−03 | 1.11E−08 | 8.30% |
| 278 | SRP2037:B05 | rhCD3E | 6.08E−04 | 2.99E−09 | 0.80% |
| 279 | SRP2037:B06 | rhCD3E | 4.10E−04 | 1.31E−09 | 1.40% |
| 280 | SRP2037:B07 | rhCD3E | 4.31E−04 | 2.49E−09 | 3.50% |
| 281 | SRP2037:B08 | rhCD3E | 3.59E−04 | 1.80E−09 | 1.40% |
| 282 | SRP2037:B09 | rhCD3E | 3.58E−04 | 1.65E−09 | 1.50% |
| 283 | SRP2037:B10 | rhCD3E | 1.06E−03 | 6.18E−09 | 3.30% |
| 284 | SRP2037:B11 | rhCD3E | 4.93E−04 | 3.44E−09 | 0.80% |
| 285 | SRP2037:C01 | rhCD3E | 1.12E−03 | 8.47E−09 | 4.30% |
| 286 | SRP2037:C02 | rhCD3E | 9.30E−04 | 5.31E−09 | 3.40% |
| 287 | SRP2037:C03 | rhCD3E | 3.71E−03 | 1.32E−06 | 0.30% |
| 288 | SRP2037:C04 | rhCD3E | 1.86E−03 | 1.80E−08 | 3.40% |
| 289 | SRP2037:C05 | rhCD3E | 4.37E−04 | 3.08E−09 | 19.10% |
| 290 | SRP2037:C06 | rhCD3E | 8.44E−05 | 4.93E−10 | 1.20% |

TABLE 15
Table 15: The results of characteristics of the SRP
2037 antibodies generated by affinity modulation
of the SRP2037-B10 using the cell binding assay.

HUT78 cell binding

| Clone | Bₘₐₓ (MFI) | K_{d} (nM) |
|---|---|---|
| 2037-B10 WT | 1,045 | 0.15 |
| 2037-B10_Hc-A33D | 103 | 8.57 |
| 2037-B10_Hc-A59D | 742 | 0.07 |
| 2037-B10_Hc-F104D | 617 | 1.72 |
| 2037-B10_Hc-F27D | 1,357 | 1.27 |
| 2037-B10_Hc-F29D | 1,080 | 1.87 |
| 2037-B10_Hc-G105D | 1,006 | 0.36 |
| 2037-B10_Hc-K31D | 1,060 | 0.94 |
| 2037-B10_Hc-K54D | 857 | 0.37 |
| 2037-B10_Hc-N103D | 166 | 28.64 |
| 2037-B10_Hc-N106D | 1,013 | 0.26 |
| 2037-B10_Hc-N30D | 989 | 0.22 |
| 2037-B10_Hc-N56D | 216 | 27.38 |
| 2037-B10_Hc-R50D | 119 | 6.61 |
| 2037-B10_Hc-R52D | 142 | 14.09 |
| 2037-B10_Hc-S107D | 1,057 | 1.72 |
| 2037-B10_Hc-Y108D | 820 | 0.12 |
| 2037-B10_Hc-Y55D | 544 | 0.27 |
| 2037-B10_Hc-Y61D | 689 | 0.32 |
| 2037-B10_Lc-N96D | 1,224 | 0.43 |
| 2037-B10_Lc-R97D | 953 | 0.58 |
| 2037-B10_Lc-S95D | 896 | 0.29 |
| 2037-B10_Lc-W93D | 88 | 21.88 |
| 2037-B10_Lc-Y94D | 1,603 | 0.7 |

MFI, mean fluorescence intensity

TABLE 16
Table 16: The results of characteristics of the SRP 2037
antibodies generated by affinity modulation of the SRP2037-
B10 using the Biacore assay to measure kinetics.

| | Biacore kinetics, human CD3 epsilon | | | |
|---|---|---|---|---|
| Ligand | ka (1/Ms) | kd (1/s) | KD (M) | Chi²/Rmax |
| 2037-B10 WT | 4.78E+05 | 2.22E−04 | 4.65E−10 | 4.10% |
| 2037-B10_Hc-A59D | 5.27E+05 | 2.51E−04 | 4.76E−10 | 4.20% |
| 2037-B10_Hc-F104D | 8.07E+04 | 6.31E−04 | 7.82E−09 | 0.80% |
| 2037-B10_Hc-F27D | 2.59E+05 | 2.78E−04 | 1.07E−09 | 1.30% |
| 2037-B10_Hc-F29D | 2.08E+05 | 4.91E−04 | 2.36E−09 | 1.40% |
| 2037-B10_Hc-G105D | 2.70E+05 | 2.07E−04 | 7.67E−10 | 3.00% |
| 2037-B10_Hc-K31D | 2.71E+05 | 3.77E−04 | 1.39E−09 | 1.10% |
| 2037-B10_Hc-K54D | 3.37E+05 | 2.89E−04 | 8.56E−10 | 1.50% |
| 2037-B10_Hc-N103D | 6.86E+03 | 5.83E−04 | 8.50E−08 | 0.40% |
| 2037-B10_Hc-N106D | 3.29E+05 | 2.77E−04 | 8.41E−10 | 1.70% |
| 2037-B10_Hc-N30D | 5.24E+05 | 1.89E−04 | 3.60E−10 | 2.70% |
| 2037-B10_Hc-N56D | 1.24E+05 | 6.68E−04 | 5.37E−09 | 0.90% |
| 2037-B10_Hc-S107D | 1.46E+05 | 2.15E−04 | 1.47E−09 | 0.20% |
| 2037-B10_Hc-Y108D | 7.96E+05 | 1.90E−04 | 2.39E−10 | 5.00% |
| 2037-B10_Hc-Y55D | 2.84E+05 | 3.72E−04 | 1.31E−09 | 0.90% |
| 2037-B10_Hc-Y61D | 3.21E+05 | 5.91E−04 | 1.84E−09 | 3.10% |
| 2037-B10_Lc-N96D | 4.45E+05 | 2.65E−04 | 5.96E−10 | 4.40% |
| 2037-B10_Lc-W93D | 7.72E+03 | 7.12E−08 | 9.23E−12 | 2.60% |

TABLE 17
Table 17: The characteristics of the SRP 2037-B10 antibodies
generated by affinity modulation of the SRP2037-B10 with
kappa grafted LCs using the cell binding assay.

| | |
|---|---|
| 2037-B10 kappa grafted | HUT78 cell binding |

| LCs | Bmax (MFI) | Kd (nM) |
|---|---|---|
| 2037-B10 wt LC (lambda) | 1030 | 0.4 |
| 2037-B10-Lc1 | 514 | 221.5 |
| 2037-B10-Lc2 | 63 | 24.0 |
| 2037-B10-Lc3 | 1036 | 62.6 |
| 2037-B10-Lc4 | 130 | 30.4 |
| 2037-B10-Lc5 | 1202 | 40.7 |
| 2037-B10-Lc6 | 1317 | 0.8 |
| 2037-B10-Lc7 | 737 | 337.9 |
| 2037-B10-Lc8 | 1246 | 0.3 |
| 2037-B10-Lc9 | 1233 | 0.4 |
| 2037-B10-Lc10 | 1238 | 0.6 |

TABLE 18
TABLE 18: The kinetic binding characteristics of the SRP
2037-B10 heavy chain paired with different light chains.

| 2037- | | | | | | | | |
|---|---|---|---|---|---|---|---|---|
| B10 HC | | | | | | | | |
| paired | | | | | | | | |
| with | Kinetic binding for human CD3 | | | | Kinetic binding for cynomolgus CD3 | | | |
| different | epsilon | | | | epsilon | | | |
| LC | | | KD | Chi² | | | | Chi² |
| Ligand | kₐ (1/Ms) | k_{d} (1/s) | (M) | (RU²) | kₐ (1/Ms) | k_{d} (1/s) | K_{D} (M) | (RU²) |
| parent | 5.14E+05 | 3.06E−04 | 5.96E−10 | 1.57 | 8.84E+05 | 3.68E−04 | 4.16E−10 | 1.14 |
| LC | | | | | | | | |
| Kappa | no binding | | | | no binding | | | |
| Lc1 | | | | | | | | |
| Kappa | no binding | | | | no binding | | | |
| Lc2 | | | | | | | | |
| Kappa | 5.50E+04 | 3.93E−04 | 7.15E−09 | 0.0539 | 5.06E+04 | 6.90E−04 | 1.36E−08 | 0.0195 |
| Lc3 | | | | | | | | |
| Kappa | no binding | | | | no binding | | | |
| Lc4 | | | | | | | | |
| Kappa | 3.82E+04 | 4.64E−04 | 1.22E−08 | 0.0165 | 5.77E+04 | 4.78E−04 | 8.28E−09 | 0.0121 |
| Lc5 | | | | | | | | |
| Kappa | 4.30E+05 | 2.78E−04 | 6.48E−10 | 2.37 | 7.53E+05 | 3.11E−04 | 4.13E−10 | 2.2 |
| Lc6 | | | | | | | | |
| Kappa | no binding | | | | no binding | | | |
| Lc7 | | | | | | | | |
| Kappa | 5.41E+05 | 2.67E−04 | 4.93E−10 | 3.38 | 9.20E+05 | 3.11E−04 | 3.38E−10 | 2.46 |
| Lc8 | | | | | | | | |
| Kappa | 5.80E+05 | 2.97E−04 | 5.11E−10 | 2.38 | 1.03E+06 | 3.40E−04 | 3.29E−10 | 2.03 |
| Lc9 | | | | | | | | |
| Kappa | 6.77E+05 | 3.14E−04 | 4.64E−10 | 2.16 | 1.09E+06 | 4.04E−04 | 3.71E−10 | 1.57 |
| Lc10 | | | | | | | | |

TABLE 19
Table 19: The cell binding characteristics of different affinity modulated
SRP 2037-B10 heavy chains paired with different light chains.

| | HUT78 Bₘₐₓ | HUT78 K_{d} (nM) |
|---|---|---|
| 2037-B10 Parent | 1887 | 2.325 |
| Parent HC + LC_R97D | 2078 | 2.928 |
| Parent HC + Kappa Lc5 | 1852 | 46.99 |
| Parent HC + Kappa Lc6 | 1780 | 1.596 |
| HC_F104D + Parent LC | 1200 | 22.96 |
| HC_F104D + LC_R97D | 2707 | 125.8 |
| HC_F104D + Kappa Lc5 | 522.8 | 226.1 |
| HC_F104D + Kappa Lc6 | 760.5 | 24.62 |
| HC_F29D + Parent LC | NSB | NSB |
| HC_F29D + LC_R97D | NSB | NSB |
| HC_F29D + Kappa Lc5 | NSB | NSB |
| HC_F29D + Kappa Lc6 | 1041 | 3.723 |
| HC_S107D + Parent LC | 1634 | 15.55 |
| HC_S107D + LC_R97D | 1804 | 38.08 |
| HC_S107D + Kappa Lc5 | 404.2 | 86.4 |
| HC_S107D + Kappa Lc6 | 1553 | 17.28 |
| HC_Y55D + Parent LC | 1274 | 9.358 |
| HC_Y55D + LC_R97D | 1188 | 62.44 |
| HC_Y55D + Kappa Lc5 | 441.5 | 174.1 |
| HC_Y55D + Kappa Lc6 | 1247 | 6.346 |
| HC_Y61D + Parent LC | 804.3 | 18.8 |
| HC_Y61D + LC_R97D | 1998 | 121.4 |
| HC_Y61D + Kappa Lc5 | 772.2 | 311.9 |
| HC_Y61D + Kappa Lc6 | 737.2 | 6.605 |

NSB = non-saturated binding

TABLE 20
TABLE 20: The cell binding characteristics of different affinity modulated
SRP 2037-B10 heavy chains paired with different light chains.

| | Kinetic binding for human CD3 | | | | Kinetic binding for cynomolgus | | | |
|---|---|---|---|---|---|---|---|---|
| | epsilon | | | | CD3 epsilon | | | |
| | kₐ | | K_{D} | Chi² | kₐ | | K_{D} | Chi² |
| Ligand | (1/Ms) | k_{d} (1/s) | (M) | (RU²) | (1/Ms) | k_{d} (1/s) | (M) | (RU²) |
| 2037-B10 parent | 2.56E+05 | 5.64E−04 | 2.20E−09 | 0.0534 | 8.24E+05 | 3.44E−04 | 4.18E−10 | 1.08 |
| HC + parent LC | | | | | | | | |
| Parent HC + | 1.72E+05 | 2.57E−04 | 1.49E−09 | 0.224 | Not determined | | | |
| LC_R97D | | | | | | | | |
| Parent HC + | 2.44E+05 | 4.79E−04 | 1.96E−09 | 0.0698 | 6.70E+05 | 3.75E−04 | 5.60E−10 | 1.39 |
| Kappa Lc6 | | | | | | | | |
| HC_F104D + | 7.12E+04 | 2.41E−04 | 3.38E−09 | 0.00589 | 1.66E+05 | 1.07E−03 | 6.45E−09 | 0.379 |
| Parent LC | | | | | | | | |
| HC_F104D + | 1.64E+04 | 1.15E−04 | 6.99E−09 | 0.0537 | Not determined | | | |
| LC_R97D | | | | | | | | |
| HC_F104D + | 1.01E+05 | 3.74E−04 | 3.70E−09 | 0.021 | 1.11E+05 | 1.65E−03 | 1.49E−08 | 0.0194 |
| Kappa Lc6 | | | | | | | | |
| HC_F29D + | 4.12E+04 | 4.53E−04 | 1.10E−08 | 0.0327 | 2.13E+05 | 5.73E−04 | 2.69E−09 | 1.01 |
| Parent LC | | | | | | | | |
| HC_F29D + | 4.11E+03 | 7.61E−04 | 1.85E−07 | 0.156 | Not determined | | | |
| LC_R97D | | | | | | | | |
| HC_F29D + | 4.05E+04 | 7.49E−04 | 1.85E−08 | 0.0118 | 2.06E+05 | 1.21E−03 | 5.87E−09 | 0.135 |
| Kappa Lc6 | | | | | | | | |
| HC_S107D + | 8.10E+04 | 4.10E−04 | 5.06E−09 | 0.00726 | 2.07E+05 | 3.40E−04 | 1.64E−09 | 0.748 |
| Parent LC | | | | | | | | |
| HC_S107D + | 3.01E+04 | 4.74E−04 | 1.58E−08 | 0.0214 | Not determined | | | |
| LC_R97D | | | | | | | | |
| HC_S107D + | 5.32E+04 | 4.59E−04 | 8.62E−09 | 0.00997 | 1.64E+05 | 5.58E−04 | 3.41E−09 | 0.0672 |
| Kappa Lc6 | | | | | | | | |
| HC_Y55D + | 1.13E+05 | 2.77E−04 | 2.45E−09 | 0.00911 | 5.79E+05 | 5.80E−04 | 1.00E−09 | 0.514 |
| Parent LC | | | | | | | | |
| HC_Y55D + | 8.70E+04 | 4.41E−04 | 5.07E−09 | 0.0233 | Not determined | | | |
| LC_R97D | | | | | | | | |
| HC_Y55D + | 1.01E+05 | 3.92E−04 | 3.89E−09 | 0.0267 | 4.51E+05 | 9.45E−04 | 2.09E−09 | 0.373 |
| Kappa Lc6 | | | | | | | | |
| HC_Y61D + | 1.11E+05 | 3.79E−04 | 3.42E−09 | 0.0124 | 7.09E+05 | 1.23E−03 | 1.73E−09 | 0.498 |
| Parent LC | | | | | | | | |
| HC_Y61D + | 7.40E+04 | 2.90E−04 | 3.91E−09 | 0.0878 | Not determined | | | |
| LC_R97D | | | | | | | | |
| HC_Y61D + | 1.02E+05 | 3.69E−04 | 3.63E−09 | 0.0375 | 3.87E+05 | 1.54E−03 | 3.99E−09 | 0.325 |
| Kappa Lc6 | | | | | | | | |

TABLE 21
Table 21: The stability characteristics of different affinity modulated
SRP 2037-B10 heavy chains paired with different light chains.

| LC | HC | Tm1 (° C.) | Tm2 (° C.) |
|---|---|---|---|
| 2037-B10 LC | 2037-B10_Hc | 59.4 | not detected |
| | HC_Y55D | 59.9 | not detected |
| | HC_Y61D | 60.1 | not detected |
| | HC_F104D | 59.4 | not detected |
| | HC_S107D | 60 | not detected |
| | HC_F29D | 57.2 | not detected |
| Kappa Lc5 | Parent HC | 62.2 | not detected |
| | HC_Y55D | 62.4 | not detected |
| | HC_Y61D | 62.7 | not detected |
| | HC_F104D | 62.2 | not detected |
| | HC_S107D | 62 | not detected |
| | HC_F29D | 60.5 | not detected |
| Kappa Lc6 | Parent HC | 62.2 | 74.2 |
| | HC_Y55D | 62.2 | 74.8 |
| | HC_Y61D | 62.4 | 74.5 |
| | HC_F104D | 62.2 | 75 |
| | HC_S107D | 62.2 | 74.6 |
| | HC_F29D | 62.4 | not detected |
| LC_R97D | Parent HC | 59 | not detected |
| | HC_Y55D | 59 | not detected |
| | HC_Y61D | 59.2 | not detected |
| | HC_F104D | 58.9 | not detected |
| | HC_S107D | 57 | not detected |
| | HC_F29D | Not determined | Not determined |

TABLE 22
Table 22: The Anti-EpCAM × anti-CD3 tandem
scFv T-cell mediated H1975 killing potency.

| | | EC50 | Span | EC50 | Span |
|---|---|---|---|---|---|
| | | (pM) | (%) | (pM) | (%) |
| Name | SP | Donor 1 | Donor 1 | Donor 2 | Donor 2 |
| aEpCAM × | SP6063 | 8 | 38 | 4 | 51 |
| hOKT3(MT110/ | | | | | |
| AMG110) | | | | | |
| aEpCAM × 2037-B10 | SP8907 | 3 | 33 | 2 | 34 |

Accordingly, this example indicates that an anti-CD3 antibody can be provided, wherein the antibody includes a heavy chain containing a V_{H} sequence comprising, consisting of, or consisting essentially of any one of SEQ ID NOs: 204-231, and wherein the light chain contains a V_{L} comprising, consisting of, or consisting essentially of any one of SEQ ID NOs: 232-255. In addition, anti-CD3 antibody can be provided, wherein the antibody includes a heavy chain containing a V_{H} sequence comprising, consisting of, or consisting essentially of any one of SEQ ID NOs: 204-231, and wherein the light chain is obtained from the light chain of any suitable antibody.

### Example 16

### Expression and Purification of Other SP34 Igg Derivatives

XpressCF+™ Protein Synthesis and analysis. 1 mL XCF reactions were performed in 24 well flower plate by mixing 37% (v/v) IAM treated XtractCF™ e251, 3 μg/mL heavy chain and light chain plasmid at various ratios, 4 Standard generic U/L T7 RNAP (XpressRNAP™) 8 mM magnesium glutamate, 10 mM ammonium glutamate, 130 mM potassium glutamate, 35 mM sodium pyruvate, 2 mM GSSG, 1.2 mM AMP, 0.86 mM each of GMP, UMP, and CMP, 2 mM amino acids (1 mM for tyrosine), 4 mM sodium oxalate, 1 mM putrescine, 1.5 mM spermidine, 15 mM potassium phosphate. The XCF reaction was incubated with shaking at 650 rpm for 14 hours in an Eppendorf Thermomixer R at 25° C. Titer of IgG expressed in XCF reactions was measured by PhyTips (PhyNexus) purification followed by A280 measurement for quantification. Protein purity (monomer %) was analyzed using size exclusion chromatography.

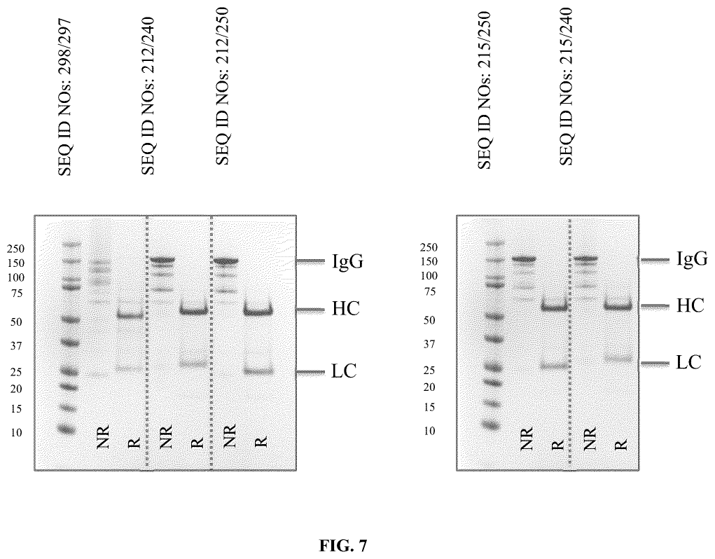

Five anti-CD3 IgGs including one Engmab benchmark (SEQ ID NOs 298/297) and four Sutro molecules (SEQ ID NOs: 212/240, 212/250, 215/250, and 215/240) were expressed using XpressCF+™ protein synthesis platform. The result and SEQ ID NOs are shown in Table 23. Antibodies 212/240 and 212/250 showed similar soluble titer to 298/297, while 212/250 and 215/240 were expressed with 35% and 58% higher titer than 298/297, respectively. In addition, all Sutro lead molecules showed superior heavy and light chain assembly. Antibody 298/297 showed 66% of monomer by size exclusion chromatograph using Zenix SEC-300 column. In contrast, Sutro lead molecules showed monomer % of 90-96%. Superior heavy chain and LC assembly of Sutro lead molecules is also observed on SDS-PAGE (FIG. 7). Assembled IgG were the dominant species on SDS-PAGE while only limited amount of IgG was detected for antibody 298/297.

Table 23 provides a summary of soluble titer and monomer % of aCD3 IgG.

| | | Phytip | Phytip |
|---|---|---|---|
| Antibody | | Titer | Capture, |
| Construct | Construct | (mg/L) | % Monomer |
| 298/297 | Engmab_83A10_3-VH_CL | 184.38 | 66.01 |
| | Engmab_83A10_1-VL_CH1_Fc | | |
| 212/240 | 2037-B10 IgG HC FH | 184.96 | 90.14 |
| | 2037-A02/2037-B10_Lc | | |
| 212/250 | 2037-B10 IgG HC FH | 248.73 | 92.35 |
| | 2037-B10-Lc6 | | |
| 215/250 | 2037-B10_Hc-S107D | 290.4 | 96.4 |
| | 2037-B10-Lc6 | | |
| 215/240 | 2037-B10_Hc-S107D | 199.44 | 94.07 |
| | 2037-A02/2037-B10_LC | | |

### Example 17

### Improved Stability of SP34 Igg Derivatives with Kappa-Grafted LC

A protein thermal shift assay was carried out by mixing the protein to be assayed with an environmentally sensitive dye (SYPRO Orange, Life Technologies Cat #S-6650) in a phosphate buffered solution (PBS) and monitoring the fluorescence of the mixture in real time as it underwent controlled thermal denaturation. Protein solutions between 0.2-2 mg/mL were mixed at a 1:1 volumetric ratio with a 1:500 PBS-diluted solution of SYPRO Orange (SYPRO Orange stock dye is 5000× in DMSO). 10 μL aliquots of the protein-dye mixture were dispensed in quadruplicate in a 384-well microplate (Bio-Rad Cat #MSP-3852), and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (˜1.5° C. per minute), allowing 3 seconds of equilibration at each temperature before taking a fluorescence measurement. At the end of the experiment, the transition melting temperatures (TM1 and TM2) were determined using the Bio-Rad CFX manager software. TM1 represents the melting temperature of the Fc domain. TM2 represents the melting temperature of the Fab domain.

Anti-CD3 SP34 derivative antibodies were expressed and purified, and their thermal stability was measured by differential scanning fluorimetry (DSF). Melting temperature transitions are reported in Table 24. Antibodies 298/297 and 211/237 both exhibited a single thermal transition at 61.4 C and 65 C, respectively. In both cases, a second thermal transition was not detected, likely reflecting a global unfolding of both Fab and Fc fragments, suggesting that the Fab fragment may be less stable than in other antibodies. Similarly, antibodies 212/240 and 215/240 both exhibited a single thermal transition. However, antibodies 212/250 and 215/250 which have kappa-grafted LCs had two thermal transitions, suggesting that the Fab fragment may be significantly stabilized by the kappa LC and unfold at a higher melting temperature.

TABLE 24

| Antibody | | Tm1 | Tm2 |
|---|---|---|---|
| Construct | Sample | (° C.) | (° C.) |
| herceptin | Herceptin | 70.3 | 80.8 |
| 298/297 | Engmab_83A10_3-VH_CL | 61.4 | ND |
| | Engmab_83A10_1-VL_CH1_Fc; | | |
| 211/237 | SP34 IgG | 65 | ND |
| 212/240 | 2037-B10 IgG | 62.6 | ND |
| 212/250 | 2037-B10 IgG Lc6 | 64.7 | 71.1 |
| 215/240 | 2037-B10 IgG S107D | 62.6 | ND |
| 215/250 | 2037-B10 IgG S107D Lc6 | 64.9 | 70.8 |

### Example 18

### Sequences

Table 25 provides sequences referred to herein.

TABLE 25

| SEQ | | | | |
|---|---|---|---|---|
| ID | | | | |
| NO: | Molecule | Region | Scheme | Sequence |
| 1 | Human (hCD3e) | | | MQSGTHWRVLGLCLLSVGVWGQDGNE |
| | UniProt: P0776; | | | EMGGITQTPYKVSISGTTVILTCPQY |
| | CD3E_HUMAN | | | PGSEILWQHNDKNIGGDEDDKNIGSD |
| | T-cell surface | | | EDHLSLKEFSELEQSGYYVCYPRGSK |
| | glycoprotein CD3 | | | PEDANFYLYLRARVCENCMEMDVMSV |
| | epsilon chain. | | | ATIVIVDICITGGLLLLVYYWSKNRK |
| | | | | AKAKPVTRGAGAGGRQRGQNKERPPP |
| | | | | VPNPDYEPIRKGQRDLYSGLNQRRI |
| 2 | Cynomolgus | | | MQSGTRWRVLGLCLLSIGVWGQDGNE |
| | CD3e | | | EMGSITQTPYQVSISGTTVILTCSQH |
| | UniProt: Q95LI5; | | | LGSEAQWQHNGKNKEDSGDRLFLPEF |
| | CD3E_MACFA | | | SEMEQSGYYVCYPRGSNPEDASHHLY |
| | T-cell surface | | | LKARVCENCMEMDVMAVATIVIVDIC |
| | glycoprotein CD3 | | | ITLGLLLLVYYWSKNRKAKAKPVTRG |
| | epsilon chain | | | AGAGGRQRGQNKERPPPVPNPDYEPI |
| | | | | RKGQQDLYSGLNQRRI |
| 3 | Murine CD3e | | | MRWNTFWGILCLSLLAVGTCQDDAEN |
| | UniProt: P22646; | | | IEYKVSISGTSVELTCPLDSDENLKW |
| | CD3E MOUSE | | | EKNGQELPQKHDKHLVLQDESEVEDS |
| | T-cell surface | | | GYYVCYTPASNKNTYLYLKARVCEYC |
| | glycoprotein CD3 | | | VEVDLTAVAIIIIVDICITLGLLMVI |
| | epsilon chain | | | YYWSKNRKAKAKPVTRGTGAGSRPRG |
| | | | | QNKERPPPVPNPDYEPIRKGQRDLYS |
| | | | | GLNQRAV |
| 4 | MSP34 | CDR-H1 | Chothia | GFTENTY |
| 5 | HSP34-HC4 | CDR-H1 | Chothia | GFTENKY |
| 6 | hSP34-Hc3 | CDR-H1 | Chothia | GFTENKY |
| 7 | hSP34-Hc2 | CDR-H1 | Chothia | GFTENKY |
| 8 | hSP34-Hc1 | CDR-H1 | Chothia | GFTENKY |
| 9 | SP34-Hc | CDR-H1 | Chothia | GFTENKY |
| 10 | 2037-B10 | CDR-H1 | Chothia | GFTENKY |
| 11 | 2037- | CDR-H1 | Chothia | GFTENKY |
| | B02/A11/A02 | | | |
| 12 | 2037-B10_VH- | CDR-H1 | Chothia | GFTENKY |
| | Y108D | | | |
| 13 | 2037-B10_VH- | CDR-H1 | Chothia | GFTENKY |
| | S107D | | | |
| 14 | 2037-B10_VH- | CDR-H1 | Chothia | GFTENKY |
| | N106D | | | |
| 15 | 2037-B10_VH- | CDR-H1 | Chothia | GFTENKY |
| | G105D | | | |
| 16 | 2037-B10_VH- | CDR-H1 | Chothia | GFTENKY |
| | F104D | | | |
| 17 | 2037-B10_VH- | CDR-H1 | Chothia | GFTENKY |
| | N103D | | | |
| 18 | 2037-B10_VH- | CDR-H1 | Chothia | GFTENKY |
| | Y61D | | | |
| 19 | 2037-B10_VH- | CDR-H1 | Chothia | GFTENKY |
| | A59D | | | |
| 20 | 2037-B10_VH- | CDR-H1 | Chothia | GFTENKY |
| | N56D | | | |
| 21 | 2037-B10_VH- | CDR-H1 | Chothia | GFTENKY |
| | Y55D | | | |
| 22 | 2037-B10_VH- | CDR-H1 | Chothia | GFTENKY |
| | K54D | | | |
| 23 | 2037-B10_VH- | CDR-H1 | Chothia | GFTENKY |
| | R52D | | | |
| 24 | 2037-B10_VH- | CDR-H1 | Chothia | GFTENKY |
| | R50D | | | |
| 25 | 2037-B10_VH- | CDR-H1 | Chothia | GFTENKY |
| | A33D | | | |
| 26 | 2037-B10_VH- | CDR-H1 | Chothia | GFTFNKY |
| | K31D | | | |
| 27 | 2037-B10_VH- | CDR-H1 | Chothia | GFTFDKY |
| | N30D | | | |
| 28 | 2037-B10_VH- | CDR-H1 | Chothia | GFTDNKY |
| | F29D | | | |
| 29 | 2037-B10_VH- | CDR-H1 | Chothia | GDTENKY |
| | F27D | | | |
| 30 | MSP34 | CDR-H1 | Kabat | TYAMN |
| 31 | HSP34-HC4 | CDR-H1 | Kabat | KYAMN |
| 32 | hSP34-Hc3 | CDR-H1 | Kabat | KYAMN |
| 33 | hSP34-Hc2 | CDR-H1 | Kabat | KYAMN |
| 34 | hSP34-Hc1 | CDR-H1 | Kabat | KYAMN |
| 35 | SP34-Hc | CDR-H1 | Kabat | KYAMN |
| 36 | 2037-B10 | CDR-H1 | Kabat | KYAMN |
| 37 | 2037- | CDR-H1 | Kabat | KYAMN |
| | B02/A11/A02 | | | |
| 38 | 2037-B10_VH- | CDR-H1 | Kabat | KYAMN |
| | Y108D | | | |
| 39 | 2037-B10_VH- | CDR-H1 | Kabat | KYAMN |
| | S107D | | | |
| 40 | 2037-B10_VH- | CDR-H1 | Kabat | KYAMN |
| | N106D | | | |
| 41 | 2037-B10_VH- | CDR-H1 | Kabat | KYAMN |
| | G105D | | | |
| 42 | 2037-B10_VH- | CDR-H1 | Kabat | KYAMN |
| | F104D | | | |
| 43 | 2037-B10_VH- | CDR-H1 | Kabat | KYAMN |
| | N103D | | | |
| 44 | 2037-B10_VH- | CDR-H1 | Kabat | KYAMN |
| | Y61D | | | |
| 45 | 2037-B10_VH- | CDR-H1 | Kabat | KYAMN |
| | A59D | | | |
| 46 | 2037-B10_VH- | CDR-H1 | Kabat | KYAMN |
| | N56D | | | |
| 47 | 2037-B10_VH- | CDR-H1 | Kabat | KYAMN |
| | Y55D | | | |
| 48 | 2037-B10_VH- | CDR-H1 | Kabat | KYAMN |
| | K54D | | | |
| 49 | 2037-B10_VH- | CDR-H1 | Kabat | KYAMN |
| | R52D | | | |
| 50 | 2037-B10_VH- | CDR-H1 | Kabat | KYAMN |
| | R50D | | | |
| 51 | 2037-B10_VH- | CDR-H1 | Kabat | KYDMN |
| | A33D | | | |
| 52 | 2037-B10_VH- | CDR-H1 | Kabat | KYAMN |
| | K31D | | | |
| 53 | 2037-B10_VH- | CDR-H1 | Kabat | KYAMN |
| | N30D | | | |
| 54 | 2037-B10_VH- | CDR-H1 | Kabat | KYAMN |
| | F29D | | | |
| 55 | 2037-B10_VH- | CDR-H1 | Kabat | KYAMN |
| | F27D | | | |
| 56 | MSP34 | CDR-H2 | Chothia | RSKYNNYA |
| 57 | HSP34-HC4 | CDR-H2 | Chothia | RSKYNNYA |
| 58 | hSP34-Hc3 | CDR-H2 | Chothia | RSKYNNYA |
| 59 | hSP34-Hc2 | CDR-H2 | Chothia | RSKYNNYA |
| 60 | hSP34-Hc1 | CDR-H2 | Chothia | RSKYNNYA |
| 61 | SP34-Hc | CDR-H2 | Chothia | RSKYNNYA |
| 62 | 2037-B10 | CDR-H2 | Chothia | RTKYNNYA |
| 63 | 2037- | CDR-H2 | Chothia | RSKYNNYA |
| | B02/A11/A02 | | | |
| 64 | 2037-B10_VH- | CDR-H2 | Chothia | RTKYNNYA |
| | Y108D | | | |
| 65 | 2037-B10_VH- | CDR-H2 | Chothia | RTKYNNYA |
| | S107D | | | |
| 66 | 2037-B10_VH- | CDR-H2 | Chothia | RTKYNNYA |
| | N106D | | | |
| 67 | 2037-B10_VH- | CDR-H2 | Chothia | RTKYNNYA |
| | G105D | | | |
| 68 | 2037-B10_VH- | CDR-H2 | Chothia | RTKYNNYA |
| | F104D | | | |
| 69 | 2037-B10_VH- | CDR-H2 | Chothia | RTKYNNYA |
| | N103D | | | |
| 70 | 2037-B10_VH- | CDR-H2 | Chothia | RTKYNNYA |
| | Y61D | | | |
| 71 | 2037-B10_VH- | CDR-H2 | Chothia | RTKYNNYA |
| | A59D | | | |
| 72 | 2037-B10_VH- | CDR-H2 | Chothia | RTKYDNYA |
| | N56D | | | |
| 73 | 2037-B10_VH- | CDR-H2 | Chothia | RTKDNNYA |
| | Y55D | | | |
| 74 | 2037-B10_VH- | CDR-H2 | Chothia | RTDYNNYA |
| | K54D | | | |
| 75 | 2037-B10_VH- | CDR-H2 | Chothia | DTKYNNYA |
| | R52D | | | |
| 76 | 2037-B10_VH- | CDR-H2 | Chothia | RTKYNNYA |
| | R50D | | | |
| 77 | 2037-B10_VH- | CDR-H2 | Chothia | RTKYNNYA |
| | A33D | | | |
| 78 | 2037-B10_VH- | CDR-H2 | Chothia | RTKYNNYA |
| | K31D | | | |
| 79 | 2037-B10_VH- | CDR-H2 | Chothia | RTKYNNYA |
| | N30D | | | |
| 80 | 2037-B10_VH- | CDR-H2 | Chothia | RTKYNNYA |
| | F29D | | | |
| 81 | 2037-B10_VH- | CDR-H2 | Chothia | RTKYNNYA |
| | F27D | | | |
| 82 | MSP34 | CDR-H2 | Kabat | RIRSKYNNYATYYADSVKD |
| 83 | HSP34-HC4 | CDR-H2 | Kabat | RIRSKYNNYATYYADSVKD |
| 84 | hSP34-Hc3 | CDR-H2 | Kabat | RIRSKYNNYATYYADSVKD |
| 85 | hSP34-Hc2 | CDR-H2 | Kabat | RIRSKYNNYATYYADSVKD |
| 86 | hSP34-Hc1 | CDR-H2 | Kabat | RIRSKYNNYATYYADSVKD |
| 87 | SP34-Hc | CDR-H2 | Kabat | RIRSKYNNYATYYADSVKD |
| 88 | 2037-B10 | CDR-H2 | Kabat | RIRTKYNNYATYYADSVKD |
| 89 | 2037- | CDR-H2 | Kabat | RIRSKYNNYATYYADSVKD |
| | B02/A11/A02 | | | |
| 90 | 2037-B10_VH- | CDR-H2 | Kabat | RIRTKYNNYATYYADSVKD |
| | Y108D | | | |
| 91 | 2037-B10_VH- | CDR-H2 | Kabat | RIRTKYNNYATYYADSVKD |
| | S107D | | | |
| 92 | 2037-B10_VH- | CDR-H2 | Kabat | RIRTKYNNYATYYADSVKD |
| | N106D | | | |
| 93 | 2037-B10_VH- | CDR-H2 | Kabat | RIRTKYNNYATYYADSVKD |
| | G105D | | | |
| 94 | 2037-B10_VH- | CDR-H2 | Kabat | RIRTKYNNYATYYADSVKD |
| | F104D | | | |
| 95 | 2037-B10_VH- | CDR-H2 | Kabat | RIRTKYNNYATYYADSVKD |
| | N103D | | | |
| 96 | 2037-B10_VH- | CDR-H2 | Kabat | RIRTKYNNYATDYADSVKD |
| | Y61D | | | |
| 97 | 2037-B10_VH- | CDR-H2 | Kabat | RIRTKYNNYDTYYADSVKD |
| | A59D | | | |
| 98 | 2037-B10_VH- | CDR-H2 | Kabat | RIRTKYDNYATYYADSVKD |
| | N56D | | | |
| 99 | 2037-B10_VH- | CDR-H2 | Kabat | RIRTKDNNYATYYADSVKD |
| | Y55D | | | |
| 100 | 2037-B10_VH- | CDR-H2 | Kabat | RIRTDYNNYATYYADSVKD |
| | K54D | | | |
| 101 | 2037-B10_VH- | CDR-H2 | Kabat | RIDTKYNNYATYYADSVKD |
| | R52D | | | |
| 102 | 2037-B10_VH- | CDR-H2 | Kabat | DIRTKYNNYATYYADSVKD |
| | R50D | | | |
| 103 | 2037-B10_VH- | CDR-H2 | Kabat | RIRTKYNNYATYYADSVKD |
| | A33D | | | |
| 104 | 2037-B10_VH- | CDR-H2 | Kabat | RIRTKYNNYATYYADSVKD |
| | K31D | | | |
| 105 | 2037-B10_VH- | CDR-H2 | Kabat | RIRTKYNNYATYYADSVKD |
| | N30D | | | |
| 106 | 2037-B10_VH- | CDR-H2 | Kabat | RIRTKYNNYATYYADSVKD |
| | F29D | | | |
| 107 | 2037-B10_VH- | CDR-H2 | Kabat | RIRTKYNNYATYYADSVKD |
| | F27D | | | |
| 108 | MSP34 | CDR-H3 | | HGNFGNSYVSWFAY |
| 109 | HSP34-HC4 | CDR-H3 | | HGNFGNSYISYWAY |
| 110 | hSP34-Hc3 | CDR-H3 | | HGNFGNSYISYWAY |
| 111 | hSP34-Hc2 | CDR-H3 | | HGNFGNSYISYWAY |
| 112 | hSP34-Hc1 | CDR-H3 | | HGNFGNSYISYWAY |
| 113 | SP34-Hc | CDR-H3 | | HGNFGNSYISYWAY |
| 114 | 2037-B10 | CDR-H3 | | HGNFGNSYISYWAY |
| 115 | 2037- | CDR-H3 | | HGNFGNSYISYWAY |
| | B02/A11/A02 | | | |
| 116 | 2037-B10_VH- | CDR-H3 | | HGNFGNSDISYWAY |
| | Y108D | | | |
| 117 | 2037-B10_VH- | CDR-H3 | | HGNFGNDYISYWAY |
| | S107D | | | |
| 118 | 2037-B10_VH- | CDR-H3 | | HGNFGDSYISYWAY |
| | N106D | | | |
| 119 | 2037-B10_VH- | CDR-H3 | | HGNFDNSYISYWAY |
| | G105D | | | |
| 120 | 2037-B10_VH- | CDR-H3 | | HGNDGNSYISYWAY |
| | F104D | | | |
| 121 | 2037-B10_VH- | CDR-H3 | | HGDEGNSYISYWAY |
| | N103D | | | |
| 122 | 2037-B10_VH- | CDR-H3 | | HGNFGNSYISYWAY |
| | Y61D | | | |
| 123 | 2037-B10_VH- | CDR-H3 | | HGNFGNSYISYWAY |
| | A59D | | | |
| 124 | 2037-B10_VH- | CDR-H3 | | HGNFGNSYISYWAY |
| | N56D | | | |
| 125 | 2037-B10_VH- | CDR-H3 | | HGNFGNSYISYWAY |
| | Y55D | | | |
| 126 | 2037-B10_VH- | CDR-H3 | | HGNFGNSYISYWAY |
| | K54D | | | |
| 127 | 2037-B10_VH- | CDR-H3 | | HGNFGNSYISYWAY |
| | R52D | | | |
| 128 | 2037-B10_VH- | CDR-H3 | | HGNFGNSYISYWAY |
| | R50D | | | |
| 129 | 2037-B10_VH- | CDR-H3 | | HGNFGNSYISYWAY |
| | A33D | | | |
| 130 | 2037-B10_VH- | CDR-H3 | | HGNFGNSYISYWAY |
| | K31D | | | |
| 131 | 2037-B10_VH- | CDR-H3 | | HGNFGNSYISYWAY |
| | N30D | | | |
| 132 | 2037-B10_VH- | CDR-H3 | | HGNFGNSYISYWAY |
| | F29D | | | |
| 133 | 2037-B10_VH- | CDR-H3 | | HGNFGNSYISYWAY |
| | F27D | | | |
| 134 | mSP34 | CDR-L1 | | RSSTGAVTTSNYAN |
| 135 | hSP34-Lc4 | CDR-L1 | | GSSTGAVTSGYYPN |
| 136 | hSP34-Lc3 | CDR-L1 | | GSSTGAVTSGYYPN |
| 137 | hSP34-Lc2 | CDR-L1 | | GSSTGAVTSGYYPN |
| 138 | hSP34-Lc1 | CDR-L1 | | GSSTGAVTSGYYPN |
| 139 | SP34-LC | CDR-L1 | | GSSTGAVTSGYYPN |
| 140 | 2037-B02 | CDR-L1 | | GSSTGAVTSGYYPN |
| 141 | 2037-A11 | CDR-L1 | | GSSTGAVTSGYYPN |
| 142 | 2037-A02/B10 | CDR-L1 | | GSSTGAVTSGYYPN |
| 143 | 2037-B10_R97D | CDR-L1 | | GSSTGAVTSGYYPN |
| 144 | 2037-B10_N96D | CDR-L1 | | GSSTGAVTSGYYPN |
| 145 | 2037-B10_S95D | CDR-L1 | | GSSTGAVTSGYYPN |
| 146 | 2037-B10_Y94D | CDR-L1 | | GSSTGAVTSGYYPN |
| 147 | 2037-B10_W93D | CDR-L1 | | GSSTGAVTSGYYPN |
| 148 | 2037-B10-Lc10 | CDR-L1 | | GSSTGAVTSGYYPN |
| 149 | 2037-B10-Lc9 | CDR-L1 | | GSSTGAVTSGYYPN |
| 150 | 2037-B10-Lc8 | CDR-L1 | | GSSTGAVTSGYYPN |
| 151 | 2037-B10-Lc7 | CDR-L1 | | GSSTGAVTSGYYPN |
| 152 | 2037-B10-Lc6 | CDR-L1 | | GSSTGAVTSGYYPN |
| 153 | 2037-B10-Lc5 | CDR-L1 | | GSSTGAVTSGYYPN |
| 154 | 2037-B10-Lc4 | CDR-L1 | | GSSTGAVTSGYYPN |
| 155 | 2037-B10-Lc3 | CDR-L1 | | GSSTGAVTSGYYPN |
| 156 | 2037-B10-Lc2 | CDR-L1 | | GSSTGAVTSGYYPN |
| 157 | 2037-B10-Lc1 | CDR-L1 | | GSSTGAVTSGYYPN |
| 158 | mSP34 | CDR-L2 | | GTNKRAP |
| 159 | hSP34-Lc4 | CDR-L2 | | GTKFLAP |
| 160 | hSP34-Lc3 | CDR-L2 | | GTKFLAP |
| 161 | hSP34-Lc2 | CDR-L2 | | GTKFLAP |
| 162 | hSP34-Lc1 | CDR-L2 | | GTKFLAP |
| 163 | SP34-LC | CDR-L2 | | GTKFLAP |
| 164 | 2037-B02 | CDR-L2 | | GTKFLAP |
| 165 | 2037-A11 | CDR-L2 | | GTKFLAP |
| 166 | 2037-A02/B10 | CDR-L2 | | GTKFLAP |
| 167 | 2037-B10_R97D | CDR-L2 | | GTKFLAP |
| 168 | 2037-B10_N96D | CDR-L2 | | GTKFLAP |
| 169 | 2037-B10_S95D | CDR-L2 | | GTKFLAP |
| 170 | 2037-B10_Y94D | CDR-L2 | | GTKFLAP |
| 171 | 2037-B10_W93D | CDR-L2 | | GTKFLAP |
| 172 | 2037-B10-Lc10 | CDR-L2 | | GTKFLAP |
| 173 | 2037-B10-Lc9 | CDR-L2 | | GTKFLAP |
| 174 | 2037-B10-Lc8 | CDR-L2 | | GTKFLAP |
| 175 | 2037-B10-Lc7 | CDR-L2 | | GTKFLAP |
| 176 | 2037-B10-Lc6 | CDR-L2 | | GTKFLAP |
| 177 | 2037-B10-Lc5 | CDR-L2 | | GTKFLAP |
| 178 | 2037-B10-Lc4 | CDR-L2 | | GTKFLAP |
| 179 | 2037-B10-Lc3 | CDR-L2 | | GTKFLAP |
| 180 | 2037-B10-Lc2 | CDR-L2 | | GTKFLAP |
| 181 | 2037-B10-Lc1 | CDR-L2 | | GTKFLAP |
| 182 | mSP34 | CDR-L3 | | ALWYSNLWV |
| 183 | hSP34-Lc4 | CDR-L3 | | ALWYSNRWV |
| 184 | hSP34-Lc3 | CDR-L3 | | ALWYSNRWV |
| 185 | hSP34-Lc2 | CDR-L3 | | ALWYSNRWV |
| 186 | hSP34-Lc1 | CDR-L3 | | ALWYSNRWV |
| 187 | SP34-LC | CDR-L3 | | ALWYSNRWV |
| 188 | 2037-B02 | CDR-L3 | | ALWYSNRWV |
| 189 | 2037-A11 | CDR-L3 | | ALWESNRWV |
| 190 | 2037-A02/B10 | CDR-L3 | | ALWYSNRWV |
| 191 | 2037-B10_R97D | CDR-L3 | | ALWYSNDWV |
| 192 | 2037-B10_N96D | CDR-L3 | | ALWYSDRWV |
| 193 | 2037-B10_S95D | CDR-L3 | | ALWYDNRWV |
| 194 | 2037-B10_Y94D | CDR-L3 | | ALWDSNRWV |
| 195 | 2037-B10_W93D | CDR-L3 | | ALDYSNRWV |
| 196 | 2037-B10-Lc10 | CDR-L3 | | ALWYSNRWV |
| 197 | 2037-B10-Lc9 | CDR-L3 | | ALWYSNRWV |
| 198 | 2037-B10-Lc8 | CDR-L3 | | ALWYSNRWV |
| 199 | 2037-B10-Lc7 | CDR-L3 | | ALWYSNRWV |
| 200 | 2037-B10-Lc6 | CDR-L3 | | ALWYSNRWV |
| 201 | 2037-B10-Lc5 | CDR-L3 | | ALWYSNRWV |
| 202 | 2037-B10-Lc4 | CDR-L3 | | ALWYSNRWV |
| 203 | 2037-B10-Lc3 | CDR-L3 | | ALWYSNRWV |
| 204 | 2037-B10-Lc2 | CDR-L3 | | ALWYSNRWV |
| 205 | 2037-B10-Lc1 | CDR-L3 | | ALWYSNRWV |
| 206 | MSP34 | VH | | EVQLVESGGGLVQPKGSLKLSCAASG |
| | | | | FTFNTYAMNWVRQAPGKGLEWVARIR |
| | | | | SKYNNYATYYADSVKDRFTISRDDSQ |
| | | | | SILYLQMNNLKTEDTAMYYCVRHGNF |
| | | | | GNSYVSWFAYWGQGTLVTVSA |
| 207 | HSP34-HC4 | VH | | EVQLVESGGGLVQPGGSLKLSCAASG |
| | | | | FTENKYAMNWVRQASGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTLVTVSS |
| 208 | hSP34-Hc3 | VH | | EVQLVESGGGLVQPGRSLRLSCTASG |
| | | | | FTFNKYAMNWFRQAPGKGLEWVARIR |
| | | | | SKYNNYATYYADSVKDRFTISRDDSK |
| | | | | SIAYLQMNSLKTEDTAVYYCVRHGNF |
| | | | | GNSYISYWAYWGQGTLVTVSS |
| 209 | hSP34-Hc2 | VH | | EVQLLESGGGLVQPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLRAEDTAVYYCVRHGNF |
| | | | | GNSYISYWAYWGQGTLVTVSS |
| 210 | hSP34-Hc1 | VH | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTLVTVSS |
| 211 | SP34-Hc | VH | | EVQLVESGGGLVQPGGSLKLSCAASG |
| | | | | FTENKYAMNWVRQAPGKGLEWVARIR |
| | | | | SKYNNYATYYADSVKDRFTISRDDSK |
| | | | | NTAYLQMNNLKTEDTAVYYCVRHGNF |
| | | | | GNSYISYWAYWGQGTLVTVSS |
| 212 | 2037-B10 | VH | | EVQLVESGGGLVKPGGSLRLSCAASG |
| | | | | FTFNKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GNSYISYWAYWGQGTPVTVSS |
| 213 | 2037- | VH | | EVQLVESGGGLVKPGGSLRLSCAASG |
| | B02/A11/A02 | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSS |
| 214 | 2037-B10_VH- | VH | | EVQLVESGGGLVKPGGSLRLSCAASG |
| | Y108D | | | FTFNKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GNSDISYWAYWGQGTPVTVSS |
| 215 | 2037-B10_VH- | VE | | EVQLVESGGGLVKPGGSLRLSCAASG |
| | S107D | | | FTENKYAMNWVRQAPGKGLEWVARIR |
| | | | | TKYNNYATYYADSVKDRFTISRDDSK |
| | | | | NTAYLQMNSLKTEDTAVYYCVRHGNF |
| | | | | GNDYISYWAYWGQGTPVTVSS |
| 216 | 2037-B10_VH- | VH | | EVQLVESGGGLVKPGGSLRLSCAASG |
| | N106D | | | FTENKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GDSYISYWAYWGQGTPVTVSS |
| 217 | 2037-B10_VH- | | | EVQLVESGGGLVKPGGSLRLSCAASG |
| | G105D | | | FTFNKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | DNSYISYWAYWGQGTPVTVSS |
| 218 | 2037-B10_VH- | VH | | EVQLVESGGGLVKPGGSLRLSCAASG |
| | F104D | | | FTFNKYAMNWVRQAPGKGLEWVARIR |
| | | | | TKYNNYATYYADSVKDRFTISRDDSK |
| | | | | NTAYLQMNSLKTEDTAVYYCVRHGND |
| | | | | GNSYISYWAYWGQGTPVTVSS |
| 219 | 2037-B10_VH- | VH | | EVQLVESGGGLVKPGGSLRLSCAASG |
| | N103D | | | FTFNKYAMNWVRQAPGKGLEWVARIR |
| | | | | TKYNNYATYYADSVKDRFTISRDDSK |
| | | | | NTAYLQMNSLKTEDTAVYYCVRHGDE |
| | | | | GNSYISYWAYWGQGTPVTVSS |
| 220 | 2037-B10_VH- | VH | | EVQLVESGGGLVKPGGSLRLSCAASG |
| | Y61D | | | FTFNKYAMNWVRQAPGKGLEWVARIR |
| | | | | TKYNNYATDYADSVKDRFTISRDDSK |
| | | | | NTAYLQMNSLKTEDTAVYYCVRHGNF |
| | | | | GNSYISYWAYWGQGTPVTVSS |
| 221 | 2037-B10_VH- | VH | | EVQLVESGGGLVKPGGSLRLSCAASG |
| | A59D | | | FTFNKYAMNWVRQAPGKGLEWVARIR |
| | | | | TKYNNYDTYYADSVKDRFTISRDDSK |
| | | | | NTAYLQMNSLKTEDTAVYYCVRHGNF |
| | | | | GNSYISYWAYWGQGTPVTVSS |
| 222 | 2037-B10_VH- | VH | | EVQLVESGGGLVKPGGSLRLSCAASG |
| | N56D | | | FTENKYAMNWVRQAPGKGLEWVARIR |
| | | | | TKYDNYATYYADSVKDRFTISRDDSK |
| | | | | NTAYLQMNSLKTEDTAVYYCVRHGNF |
| | | | | GNSYISYWAYWGQGTPVTVSS |
| 223 | 2037-B10_VH- | VH | | EVQLVESGGGLVKPGGSLRLSCAASG |
| | Y55D | | | FTENKYAMNWVRQAPGKGLEWVARIR |
| | | | | TKDNNYATYYADSVKDRFTISRDDSK |
| | | | | NTAYLQMNSLKTEDTAVYYCVRHGNF |
| | | | | GNSYISYWAYWGQGTPVTVSS |
| 224 | 2037-B10_VH- | VH | | EVQLVESGGGLVKPGGSLRLSCAASG |
| | K54D | | | FTENKYAMNWVRQAPGKGLEWVARIR |
| | | | | TDYNNYATYYADSVKDRFTISRDDSK |
| | | | | NTAYLQMNSLKTEDTAVYYCVRHGNF |
| | | | | GNSYISYWAYWGQGTPVTVSS |
| 225 | 2037-B10_VH- | VH | | EVQLVESGGGLVKPGGSLRLSCAASG |
| | R52D | | | FTFNKYAMNWVRQAPGKGLEWVARID |
| | | | | TKYNNYATYYADSVKDRFTISRDDSK |
| | | | | NTAYLQMNSLKTEDTAVYYCVRHGNF |
| | | | | GNSYISYWAYWGQGTPVTVSS |
| 226 | 2037-B10_VH- | VH | | EVQLVESGGGLVKPGGSLRLSCAASG |
| | R50D | | | FTFNKYAMNWVRQAPGKGLEWVADIR |
| | | | | TKYNNYATYYADSVKDRFTISRDDSK |
| | | | | NTAYLQMNSLKTEDTAVYYCVRHGNE |
| | | | | GNSYISYWAYWGQGTPVTVSS |
| 227 | 2037-B10_VH- | VH | | EVQLVESGGGLVKPGGSLRLSCAASG |
| | A33D | | | FTFNKYDMNWVRQAPGKGLEWVARIR |
| | | | | TKYNYATYYADSVKDRFTISRDDSKN |
| | | | | TAYLQMNSLKTEDTAVYYCVRHGNFG |
| | | | | NSYISYWAYWGQGTPVTVSS |
| 228 | 2037-B10_VH- | VH | | EVQLVESGGGLVKPGGSLRLSCAASG |
| | K31D | | | FTFNDYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GNSYISYWAYWGQGTPVTVSS |
| 229 | 2037-B10_VH- | VH | | EVQLVESGGGLVKPGGSLRLSCAASG |
| | N30D | | | FTFDKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GNSYISYWAYWGQGTPVTVSS |
| 230 | 2037-B10_VH- | VH | | EVQLVESGGGLVKPGGSLRLSCAASG |
| | F29D | | | FTDNKYAMNWVRQAPGKGLEWVARIR |
| | | | | TKYNNYATYYADSVKDRFTISRDDSK |
| | | | | NTAYLQMNSLKTEDTAVYYCVRHGNF |
| | | | | GNSYISYWAYWGQGTPVTVSS |
| 231 | 2037-B10_VH- | VH | | EVQLVESGGGLVKPGGSLRLSCAASG |
| | F27D | | | DTFNKYAMNWVRQAPGKGLEWVARIR |
| | | | | TKYNNYATYYADSVKDRFTISRDDSK |
| | | | | NTAYLQMNSLKTEDTAVYYCVRHGNE |
| | | | | GNSYISYWAYWGQGTPVTVSS |
| 232 | mSP34-VL | VL | | QAVVTQESALTTSPGETVTLTCRSST |
| | | | | GAVTTSNYANWVQEKPDHLFTGLIGG |
| | | | | TNKRAPGVPARFSGSLIGDKAALTIT |
| | | | | GAQTEDEAIYFCALWYSNLWVEGGGT |
| | | | | KLTVL |
| 233 | hSP34-Lc4-VL | VL | | QTVVTQEPSFSVSPGGTVTLTCGSST |
| | | | | GAVTSGYYPNWYQQTPGQAPRGLIGG |
| | | | | TKFLAPGVPDRESGSLLGNKAALTIT |
| | | | | GAQADDESDYYCALWYSNRWVFGGGT |
| | | | | QLTVT |
| 234 | hSP34-Lc3-VL | VL | | QTVVTQEPSLTVSPGGTVTLTCGSST |
| | | | | GAVTSGYYPNWFQQKPGQAPRGLIGG |
| | | | | TKFLAPGTPARFSGSLLGGKAALTLS |
| | | | | GVQPEDEAEYYCALWYSNRWVEGGGT |
| | | | | QLTVT |
| 235 | hSP34-Lc2-VL | VL | | QSAVTQPRSVSGSPGQSVTISCGSST |
| | | | | GAVTSGYYPNWYQQHPGKAPKGMIGG |
| | | | | TKFLAPGVPDRESGSLLGNTASLTIS |
| | | | | GLQAEDEADYYCALWYSNRWVEGGGT |
| | | | | QLTVT |
| 236 | hSP34-Lc1-VL | VL | | EIVVTQSPGTLSLSPGERATLSCGSS |
| | | | | TGAVTSGYYPNWYQQKPGQAPRGLIG |
| | | | | GTKFLAPGIPDRESGSLLGTDATLTI |
| | | | | SRLEPEDFAVYYCALWYSNRWVFGQG |
| | | | | TKLEIK |
| 237 | SP34-LC-VL | VI | | QTVVTQEPSLTVSPGGTVTLTCGSST |
| | | | | GAVTSGYYPNWVQQKPGQAPRGLIGG |
| | | | | TKFLAPGTPARFSGSLLGGKAALTLS |
| | | | | GVQPEDEAEYYCALWYSNRWVEGGGT |
| | | | | QLTVT |
| 238 | 2037-B02-VL | VL | | QTVVTQEPSLTVSPGGTVTLTCGSST |
| | | | | GAVTSGYYPNWLQQKPGQAPRGLIGG |
| | | | | TKFLAPGTPARFSGSLLGGKAALTLS |
| | | | | GVQPEDEAEYYCALWYSNRWVFGSGT |
| | | | | QLTVT |
| 239 | 2037-A11-VL | VL | | QTVVTQEPSLTVSPGGTVTLTCGSST |
| | | | | GAVTSGYYPNWLQQKPGQAPRGLIGG |
| | | | | TKFLAPGTPARESGSLLGGKAALTLS |
| | | | | GVQPEDEAEYYCALWFSNRWVFGSGT |
| | | | | QLTVT |
| 240 | 2037-A02/B10- | VL | | QTVVTQEPSLTVSPGGTVTLTCGSST |
| | VL | | | GAVTSGYYPNWLQQKPGQAPRGLIGG |
| | | | | TKFLAPGTPARFSGSLLGGKAALTLS |
| | | | | GVQPEDEAEYYCALWYSNRWVEGGGT |
| | | | | QLTVT |
| 241 | 2037-B10_VL- | VI | | QTVVTQEPSLTVSPGGTVTLTCGSST |
| | R97D | | | GAVTSGYYPNWLQQKPGQAPRGLIGG |
| | | | | TKFLAPGTPARFSGSLLGGKAALTLS |
| | | | | GVQPEDEAEYYCALWYSNDWVEGGGT |
| | | | | QLTVT |
| 242 | 2037-B10_VL- | VL | | QTVVTQEPSLTVSPGGTVTLTCGSST |
| | N96D | | | GAVTSGYYPNWLQQKPGQAPRGLIGG |
| | | | | TKFLAPGTPARFSGSLLGGKAALTLS |
| | | | | GVQPEDEAEYYCALWYSDRWVEGGGT |
| | | | | QLTVT |
| 243 | 2037-B10_VL- | VL | | QTVVTQEPSLTVSPGGTVTLTCGSST |
| | S95D | | | GAVTSGYYPNWLQQKPGQAPRGLIGG |
| | | | | TKFLAPGTPARFSGSLLGGKAALTLS |
| | | | | GVQPEDEAEYYCALWYDNRWVEGGGT |
| | | | | QLTVT |
| 244 | 2037-B10_VL- | VL | | QTVVTQEPSLTVSPGGTVTLTCGSST |
| | Y94D | | | GAVTSGYYPNWLQQKPGQAPRGLIGG |
| | | | | TKFLAPGTPARFSGSLLGGKAALTLS |
| | | | | GVQPEDEAEYYCALWDSNRWVEGGGT |
| | | | | QLTVT |
| 245 | 2037-B10_VL- | VI | | QTVVTQEPSLTVSPGGTVTLTCGSST |
| | W93D | | | GAVTSGYYPNWLQQKPGQAPRGLIGG |
| | | | | TKFLAPGTPARFSGSLLGGKAALTLS |
| | | | | GVQPEDEAEYYCALDYSNRWVEGGGT |
| | | | | QLTVT |
| 246 | 2037-B10-Lc10- | VL | | DTQVTQSPSLSASVGDRVTITCGSST |
| | VL | | | GAVTSGYYPNWLQQKPGQAPRGLIGG |
| | | | | TKFLAPGVPSRESGSLLGGKAALTIS |
| | | | | SLQPEDFAEYYCALWYSNRWVEGGGT |
| | | | | KVEIK |
| 247 | 2037-B10-Lc9- | VL | | DTQVTQSPSLSASVGDRVTITCGSST |
| | VL | | | GAVTSGYYPNWLQQKPGQAPRGLIGG |
| | | | | TKFLAPGVPSRFSGSLLGGKAALTIS |
| | | | | SLQPEDFATYYCALWYSNRWVEGGGT |
| | | | | KVEIK |
| 248 | 2037-B10-Lc8- | VL | | DTQVTQSPSLSASVGDRVTITCGSST |
| | VL | | | GAVTSGYYPNWLQQKPGQAPRGLIGG |
| | | | | TKFLAPGVPSRFSGSLSGGKATLTIS |
| | | | | SLQPEDFATYYCALWYSNRWVFGGGT |
| | | | | KVEIK |
| 249 | 2037-B10-Lc7- | VL | | DTQVTQSPSLSASVGDRVTITCGSST |
| | VL | | | GAVTSGYYPNWYQQKPGKAPKLLIGG |
| | | | | TKFLAPGVPSRFSGSLSGGKATLTIS |
| | | | | SLQPEDFATYYCALWYSNRWVEGGGT |
| | | | | KVEIK |
| 250 | 2037-B10-Lc6- | VL | | DIQMTQSPSSLSASVGDRVTITCGSS |
| | VL | | | TGAVTSGYYPNWLQQKPGQAPRGLIG |
| | | | | GTKFLAPGVPSRFSGSLSGGKATLTI |
| | | | | SSLQPEDFATYYCALWYSNRWVEGGG |
| | | | | TKVEIK |
| 251 | 2037-B10-Lc5- | VI | | DTQVTQSPSLSASVGDRVTITCGSST |
| | VL | | | GAVTSGYYPNWLQQKPGQAPRGLIGG |
| | | | | TKFLAPGVPSRFSGSGSGTDFTLTIS |
| | | | | SLQPEDFATYYCALWYSNRWVEGGGT |
| | | | | KVEIK |
| 252 | 2037-B10-Lc4- | VL | | DIQMTQSPSSLSASVGDRVTITCGSS |
| | VL | | | TGAVTSGYYPNWYQQKPGKAPKLLIG |
| | | | | GTKFLAPGVPSRFSGSLSGGKATLTI |
| | | | | SSLQPEDFATYYCALWYSNRWVEGGG |
| | | | | TKVEIK |
| 253 | 2037-B10-Lc3- | VL | | DIQMTQSPSSLSASVGDRVTITCGSS |
| | VL | | | TGAVTSGYYPNWLQQKPGQAPRGLIG |
| | | | | GTKFLAPGVPSRFSGSGSGTDETLTI |
| | | | | SSLQPEDFATYYCALWYSNRWVEGGG |
| | | | | TKVEIK |
| 254 | 2037-B10-Lc2- | VL | | DTQVTQSPSLSASVGDRVTITCGSST |
| | VL | | | GAVTSGYYPNWYQQKPGKAPKLLIGG |
| | | | | TKFLAPGVPSRESGSGSGTDETLTIS |
| | | | | SLQPEDFATYYCALWYSNRWVEGGGT |
| | | | | KVEIK |
| 255 | 2037-B10-Lc1- | VI | | DIQMTQSPSSLSASVGDRVTITCGSS |
| | VL | | | TGAVTSGYYPNWYQQKPGKAPKLLIG |
| | | | | GTKFLAPGVPSRFSGSGSGTDFTLTI |
| | | | | SSLQPEDFATYYCALWYSNRWVFGGG |
| | | | | TKVEIK |
| 256 | Human IgG1 HC | | | ASTKGPSVFPLAPSSKSTSGGTAALG |
| | Constant | | | CLVKDYFPEPVTVSWNSGALTSGVHT |
| | | | | FPAVLQSSGLYSLSSVVTVPSSSLGT |
| | | | | QTYICNVNHKPSNTKVDKKVEPKSCD |
| | | | | KTHTCPPCPAPELLGGPSVFLFPPKP |
| | | | | KDTLMISRTPEVTCVVVDVSHEDPEV |
| | | | | KFNWYVDGVEVHNAKTKPREEQYNST |
| | | | | YRVVSVLTVLHQDWLNGKEYKCKVSN |
| | | | | KALPAPIEKTISKAKGQPREPQVYTL |
| | | | | PPSREEMTKNQVSLTCLVKGFYPSDI |
| | | | | AVEWESNGQPENNYKTTPPVLDSDGS |
| | | | | FFLYSKLTVDKSRWQQGNVFSCSVMH |
| | | | | EALHNHYTQKSLSLSPGK |
| 257 | Human IgG LC | | | RTVAAPSVFIFPPSDEQLKSGTASVV |
| | Constant kappa | | | CLLNNFYPREAKVQWKVDNALQSGNS |
| | | | | QESVTEQDSKDSTYSLSSTLTLSKAD |
| | | | | YEKHKVYACEVTHQGLSSPVTKSENR |
| | | | | GEC |
| 258 | Human Lambda | | | GQPKAAPSVTLFPPSSEELQANKATL |
| | LD | | | VCLISDFYPGAVTVAWKADSSPVKAG |
| | | | | VETTTPSKQSNNKYAASSYLSLTPEQ |
| | | | | WKSHRSYSCQVTHEGSTVEKTVAPTE |
| | | | | CS |
| 259 | Fc-knob | | | MDKTHTCPPCPAPELLGGPSVELFPP |
| | | | | KPKDTLMISRTPEVTCVVVDVSHEDP |
| | | | | EVKENWYVDGVEVHNAKTKPREEQYN |
| | | | | STYRVVSVLTVLHQDWLNGKEYKCKV |
| | | | | SNKALPAPIEKTISKAKGQPREPQVY |
| | | | | TLPPSREEMTKNQVSLWCLVKGFYPS |
| | | | | DIAVEWESNGQPENNYKTTPPVLDSD |
| | | | | GSFFLYSKLTVDKSRWQQGNVESCSV |
| | | | | MHEALHNHYTQKSLSLSPGK |
| 260 | FlagHis Tag | | | GSGDYKDDDDKGSGHHHHHH |
| 261 | MycHis | | | GSGEQKLISEEDLGSGHHHHHH |
| 262 | aEpCAM x | | | MELVMTQSPSSLTVTAGEKVTMSCKS |
| | hOKT3 | | | SQSLLNSGNQKNYLTWYQQKPGQPPK |
| | (MT110/AMG110) | | | LLIYWASTRESGVPDRFTGSGSGTDE |
| | | | | TLTISSVQAEDLAVYYCQNDYSYPLT |
| | | | | FGAGTKLEIKGGGGSGGGGSGGGGSE |
| | | | | VQLLEQSGAELVRPGTSVKISCKASG |
| | | | | YAFTNYWLGWVKQRPGHGLEWIGDIF |
| | | | | PGSGNIHYNEKFKGKATLTADKSSST |
| | | | | AYMQLSSLTFEDSAVYFCARLRNWDE |
| | | | | PMDYWGQGTTVTVSSGGGGSDVQLVQ |
| | | | | SGAEVKKPGASVKVSCKASGYTFTRY |
| | | | | TMHWVRQAPGQGLEWIGYINPSRGYT |
| | | | | NYADSVKGRFTITTDKSTSTAYMELS |
| | | | | SLRSEDTATYYCARYYDDHYCLDYWG |
| | | | | QGTTVTVSSGEGTSTGSGGSGGSGGA |
| | | | | DDIVLTQSPATLSLSPGERATLSCRA |
| | | | | SQSVSYMNWYQQKPGKAPKRWIYDTS |
| | | | | KVASGVPARFSGSGSGTDYSLTINSL |
| | | | | EAEDAATYYCQQWSSNPLTFGGGTKV |
| | | | | EIK |
| 263 | aEpCAM x | | | MELVMTQSPSSLTVTAGEKVTMSCKS |
| | 2037-B10 | | | SQSLLNSGNQKNYLTWYQQKPGQPPK |
| | | | | LLIYWASTRESGVPDRFTGSGSGTDE |
| | | | | TLTISSVQAEDLAVYYCQNDYSYPLT |
| | | | | FGAGTKLEIKGGGGSGGGGSGGGGSE |
| | | | | VQLLEQSGAELVRPGTSVKISCKASG |
| | | | | YAFTNYWLGWVKQRPGHGLEWIGDIE |
| | | | | PGSGNIHYNEKFKGKATLTADKSSST |
| | | | | AYMQLSSLTFEDSAVYFCARLRNWDE |
| | | | | PMDYWGQGTTVTVSSGGGGSEVQLVE |
| | | | | SGGGLVKPGGSLRLSCAASGFTENKY |
| | | | | AMNWVRQAPGKGLEWVARIRTKYNNY |
| | | | | ATYYADSVKDRFTISRDDSKNTAYLQ |
| | | | | MNSLKTEDTAVYYCVRHGNEGNSYIS |
| | | | | YWAYWGQGTPVTVSSGGGGSGGDGSG |
| | | | | GGGSQTVVTQEPSLTVSPGGTVTLTC |
| | | | | GSSTGAVTSGYYPNWLQQKPGQAPRG |
| | | | | LIGGTKFLAPGTPARFSGSLLGGKAA |
| | | | | LTLSGVQPEDEAEYYCALWYSNRWVF |
| | | | | GGGTQLTVTHHHHHH |
| 264 | SRP2037: A01 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPDTPARFSGSL |
| | | | | LGGKAALTLSGVQPEDEAEYYCALWD |
| | | | | STRWVFGGGTQLTVT |
| 265 | SRP2037: A02 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 266 | SRP2037: A03 | scFv | | EVQLVESGGGLVKPGGSLRLSCAASG |
| | | | | FTFSKYAMNWVRQAPGKGLEWVARIR |
| | | | | SKYNNYATYYADSVKDRFTISRDDSK |
| | | | | NTAYLQINSLKTEDTAVYYCVRHGNF |
| | | | | GNSYTSYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 267 | SRP2037: A04 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | STAYLQMNSLKTEDTAVYYCARHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYHPNWLQQKP |
| | | | | GQAPRGLIGGTKFLAPGTPARESGSL |
| | | | | LGGKAALTLSGVQPEDEAEYYCALWC |
| | | | | SNRWVFGGGTQLTVT |
| 268 | >SRP2037: A05 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSR |
| | | | | NTAYLQMNSLKAEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 269 | SRP2037: A06 | scFv | | EVQLVESGGGLVKPGDSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARESGSL |
| | | | | LGGKAALTLSGVQPEDEAEYYCALWY |
| | | | | SNRWVFGGGTQLTVT |
| 270 | SRP2037: A07 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARLSGSL |
| | | | | LGGKAALTLSGVQPEDEAEYYCALWY |
| | | | | SNRWVFGGGTQLTVT |
| 271 | SRP2037: A08 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARESGSL |
| | | | | LGGKAALTLSGVQPEDEAEYYCALWY |
| | | | | SNRWVFGSGTQLTVT |
| 272 | SRP2037: A09 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 273 | SRP2037: A11 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWE |
| | | | | SNRWVFGSGTQLTVT |
| 274 | SRP2037: B01 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARESGSL |
| | | | | LGGKAALTLSGVQPEDEAAYHCALWY |
| | | | | SNRWVFGGGTQLTVT |
| 275 | SRP2037: B02 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGSGTQLTVT |
| 276 | SRP2037: B03 | scFv | | EVQLVESGGGLVKPGGGLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 277 | SRP2037: B04 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGCYPNWLQQKP |
| | | | | GQAPRGLIGGTKFLAPGTPARFSGSL |
| | | | | LGGKAALZLSGVQPEDEAEYYCALWY |
| | | | | SNRWVFGGGTQLTVT |
| 278 | SRP2037: B05 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWD |
| | | | | SNRWVFGGGTQLTVT |
| 279 | SRP2037: B06 | scFv | | EVQLVESGGGLVKPGGSLRLSCAASG |
| | | | | FTENKYAMNWVRQAPGKGLEWVARIR |
| | | | | SKYNNYETYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGDGTQLTVT |
| 280 | SRP2037: B07 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVLSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 281 | SRP2037: B08 | scFv | | EVQLVESGGGLVRPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCVLWY |
| | | | | SNRWVFGGGTQLTVT |
| 282 | SRP2037: B09 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGT |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVRPEDEAEYYCALWQ |
| | | | | SNRWZFGGGTQLTVT |
| 283 | SRP2037: B10 | scFv | | EVQLVESGGGLVKPGGSLRLSCAASG |
| | | | | FTENKYAMNWVRQAPGKGLEWVARIR |
| | | | | TKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARESGSL |
| | | | | LGGKAALTLSGVQPEDEAEYYCALWY |
| | | | | SNRWVFGGGTQLTVT |
| 284 | SRP2037: B11 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGGGTQLTVT |
| 285 | SRP2037: C01 | scFv | | EVQLVESGGGLVKPGGSLRLSCVASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | SNRWVFGSGTQLTVT |
| 286 | SRP2037: C02 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAZYYCALWQ |
| | | | | SNRWVFGGGTQLTVT |
| 287 | SRP2037: C03 | scFv | | EVQLVESGGGLVKPGGSLRLSCVASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNE |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGIKFLAPGTPARFSGSL |
| | | | | LGGKAALTLSGVQPEDEAEYYCALWY |
| | | | | SNRWVFGGGTQLTVT |
| 288 | SRP2037: C04 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWS |
| | | | | ATAGWFGGGTQLTVT |
| 289 | SRP2037: C05 | scFv | | EVQLVESGGGLVKPGGSLRLSCAASG |
| | | | | FTENKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALTLSGVQPEDEAEYYCALWY |
| | | | | STVGWFGGGTQLTVT |
| 290 | SRP2037: C06 | scFv | | EVQLVESGGGLVKPGGSLRLSCAASG |
| | | | | FTFNKYAMNWVRQAPGKGLEWVARIR |
| | | | | SKYNNYATYYADSVKDRFTISRDDSK |
| | | | | NTAYLQMNSLKTEDTAVYYCVRHGNF |
| | | | | GNSYISYWAYWGQGTPVTVSSGGGGS |
| | | | | GGDGSGGGGSQTVVTQEPSLTVSPGG |
| | | | | TVTLTCGSSTGAVTSGYYPNWLQQKP |
| | | | | GQAPRGLIGGTKFLAPGTPARFSGSL |
| | | | | LGGKAALZLSGVQPEDEAEYYCALWY |
| | | | | SNRWVFGGGTQLTVT |
| 291 | exemplary scFv- | | | AAGSDQEPKSS |
| | Fc linker | | | |
| 292 | C-terminal | | | GSGDYKDDDDKGSGHHHHHH |
| | FlagHis tag | | | |
| 293 | Linker | | | GGGGSGGGGSGGGGS |
| 297 | Engmab_83A10 | | | MQAVVTQEPSLTVSPGGTVTLTCGSS |
| | 1-VL_CH1_Fc | | | TGAVTTSNYANWVQEKPGQAFRGLIG |
| | | | | GTNKRAPGTPARFSGSLLGGKAALTL |
| | | | | SGAQPEDEAEYYCALWYSNLWVFGGG |
| | | | | TKLTVLSSASTKGPSVFPLAPSSKST |
| | | | | SGGTAALGCLVKDYFPEPVTVSWNSG |
| | | | | ALTSGVHTFPAVLQSSGLYSLSSVVT |
| | | | | VPSSSLGTQTYICNVNHKPSNTKVDK |
| | | | | KVEPKSCDKTHTCPPCPAPELLGGPS |
| | | | | VFLFPPKPKDTLMISRTPEVTCVVVD |
| | | | | VSHEDPEVKENWYVDGVEVHNAKTKP |
| | | | | REEQYNSTYRVVSVLTVLHQDWLNGK |
| | | | | EYKCKVSNKALPAPIEKTISKAKGQP |
| | | | | REPQVYTLPPSREEMTKNQVSLTCLV |
| | | | | KGFYPSDIAVEWESNGQPENNYKTTP |
| | | | | PVLDSDGSFFLYSKLTVDKSRWQQGN |
| | | | | VFSCSVMHEALHNHYTQKSLSLSPGK |
| 298 | aCD3- | | | MEVQLLESGGGLVQPGGSLRLSCAAS |
| | Engmab_83A10 | | | GFTESTYAMNWVRQAPGKGLEWVSRI |
| | 3-VH CL | | | RSKYNNYATYYADSVKGRFTISRDDS |
| | | | | KNTLYLQMNSLRAEDTAVYYCVRHGN |
| | | | | FGNSYVSWFAYWGQGTLVTVSSASVA |
| | | | | APSVEIFPPSDEQLKSGTASVVCLLN |
| | | | | NFYPREAKVQWKVDNALQSGNSQESV |
| | | | | TEQDSKDSTYSLSSTLTLSKADYEKH |
| | | | | KVYACEVTHQGLSSPVTKSENRGEC |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### REFERENCES

1. Heckman, K. L. & Pease, L. R. Gene splicing and mutagenesis by PCR-driven overlap extension. Nat. Protoc. 2, 924-932 (2007).
2. Hanes, J. & Plückthun, A. In vitro selection and evolution of functional proteins by using ribosome display. Proc. Natl. Acad. Sci. U.S.A. 94, 4937-4942 (1997).
3. Stafford, R. L. et al, In vitro Fab display—A cell-free system for IgG discovery. Protein Eng. Des. Sel. 4, 97-109 (2014).
4. Dreier, B. & Plückthun, A. Ribosome display—a technology for selecting and evolving proteins from large libraries. Methods Mol. Biol. Clifton NJ 687, 283-306 (2011).
5. Yin, G. et al. Aglycosylated antibodies and antibody fragments produced in a scalable in vitro transcription-translation system. mAbs 4 (2012).
6. Rajan, S. & Sidhu S. S. Simplified synthetic antibody libraries. Methods Enzymol. 502, 3-23 (2012).
7. Marks, J. D. & Bradbury A. Selection of human antibodies from phage display libraries. Methods Mol Biol. 248, 161-76 (2004)
8. Kivi, G. et al. HybriFree: a robust and rapid method for the development of monoclonal antibodies from different host species. BMC Biotech 16, 2 (2014).
9. T. Kuramochi, T. Igawa, H. Tsunoda, and K. Hattori, Human Monoclonal Antibodies: Methods and Protocols. Method in Molecular Biology, 1060, 123-137 (2014).
10. Cai, Q. et al A simplified and robust protocol for immunoglobulin expression in Escherichia coli cell-free protein synthesis. Biotechnol Prg 3, 823-831 (2015).
11. Smith-Garvin, et al., T Cell Activation, Annu Rev Immunology. 2009; 27: 591-619 (2009).

1. An isolated antibody that specifically binds to CD3, wherein the antibody comprises three heavy chain CDRs from a V_{H} sequence selected from SEQ ID NOs:207-231, or variants thereof, and three light chain CDRs from a V_{L} sequence selected from SEQ ID NOs:233-255, or variants thereof.

2.-57. (canceled)
